(19) United States
(12) Patent Application Publication
(10) Pub. No.: US 2022/0275092 A1
(43) Pub. Date: Sep. 1, 2022

(54) DOSAGE REGIMES FOR THE ADMINISTRATION OF A LAG-3/PD-L1 BISPECIFIC ANTIBODY

(71) Applicant: F-star Therapeutics Limited, Cambridge, Cambridgeshire (GB)

(72) Inventors: Michelle Morrow, Cambridge, Cambridgeshire (GB); Fiona Germaschewski, Cambridge, Cambridgeshire (GB); Daniel Gliddon, Cambridge, Cambridgeshire (GB); Kin-mei Leung, Cambridge (GB); Cristian Gradinaru, Cambridge, Cambridgeshire (GB); Christopher Shepherd, Cambridge, Cambridgeshire (GB); Josefin-Beate Holz, Mtinchen (DE); Louis Kayitalire, Cambridge, Cambridgeshire (GB)

(73) Assignee: F-star Therapeutics Limited, Cambridge, Cambridgeshire (GB)

(21) Appl. No.: 17/610,873

(22) PCT Filed: May 14, 2020
(86) PCT No.: PCT/EP2020/063529

§ 371 Date: Nov. 12, 2021

(30) Foreign Application Priority Data
May 14, 2019 (GB) 1906807.1
Sep. 30, 2019 (GB) 1914040.9
Jan. 9, 2020 (GB) 2000318.2

(51) Int. Cl. C07K 16/28; A61P 35/00

(52) U.S. Cl. CPC C07K 16/2827; C07K 16/2803; A61P 35/00; C07K 2317/56; C07K 2317/31; A61K 2039/545

(57) ABSTRACT

The application relates to dosage regimes for the administration of an antibody molecule which binds programmed death-ligand 1 (PD-L1) and lymphocyte-activation gene 3 (LAG-3) and their medical use in the treatment of cancer in human patients.

### FIELD OF THE INVENTION

The present invention relates to dosage regimes for the administration of an antibody molecule which binds programmed death-ligand 1 (PD-L1) and lymphocyte-activation gene 3 (LAG-3) and their medical use in the treatment of cancer in human patients. The invention further provides prognostic thresholds for predicting the likelihood of response of a human patient to the antibody.

### BACKGROUND TO THE INVENTION

Cancer is a complex disease for which there is still significant unmet need. The interplay between the host immune system and the tumour has been an area of intense non-clinical and clinical assessment in recent years. Tumour infiltrating lymphocytes (TILs) have the capacity to control the growth of tumour cells, and there is emerging clinical evidence that patients with increased TILs have a favorable prognosis. Overall, T cells play a major role in immune defense against cancer and regulation of T-cell activation is mediated by a complex interplay of stimulatory and inhibitory ligand-receptor interactions between T cells, tumour cells, and the tumour microenvironment, where tumour cells act as critical mediators of immunosuppression.

Development of immune checkpoint inhibitors, which counter-act the immunosuppressive activity of tumour cells, represents a rapidly growing avenue of treatment in clinical oncology practice, and immune checkpoint inhibitors targeting PD-1/PD-L1 (PD-1 ligand) have demonstrated some remarkable evidence of anti-tumour activity. About 20% of patients treated with monotherapy achieve clinical benefit with deep and durable responses. However, the majority of patients appear to require a combinatorial approach to overcome primary, adaptive, and acquired resistance to cancer immunotherapy, whereby primary resistance has been classified as cancers that never respond to the therapy and thus progress, acquired resistance has been classified as cancers which ultimately progress despite initially responding to the therapy and adaptive resistance has been classified as cancers that evolve resistance mechanisms to the therapy which may exhibit clinically as primary or acquired resistance (Sharma et al., 2017). The establishment of resistance to PD-1/PD-L1 therapy is complex and multi-factorial comprising environmental and genetic factors, individual history of disease, as well as the effect of previous therapies (Sharma et al., 2017).

Lymphocyte-activation gene 3 (LAG-3) is one of the key mediators of primary and potentially acquired resistance to immune checkpoint inhibitors, and first antibody combination studies in heavily pre-treated advanced melanoma patients who were relapsed or refractory to anti-PD-1/PD-L1 therapy showed early evidence of overcoming of PD-(L)1 resistance in this population.

A superior approach to co-administration of monospecific anti-PD-1/PD-L1 and anti-LAG-3 antibodies is described in WO2017/220569 A1 (F-star Delta Limited), which discloses bispecific antibodies encompassing binding sites for both PD-L1 and LAG-3, including antibody FS118, for the treatment of cancer. FS118 is a bispecific IgG1 (148,247 Da) monoclonal antibody comprising a LAG-3 antigen binding site in the Fc region and a Fab binding site for PD-L1, and that targets both human PD-L1 (hPD-L1) and human LAG-3 (hLAG-3) with comparably high affinity and exhibits blockade of LAG-3 and PD-L1-mediated inhibition of T-cell activation. This feature, and the ability to enhance bridging between T cells and tumour cells via dual targeting of LAG-3 and PD-L1, as well as to localize within the tumour microenvironment, are the unique attributes of FS118 which are expected to drive its potent anti-tumour activity.

Specifically, activated T cells in the lymph nodes express LAG-3 and anti-LAG-3/PD-L1 bispecific antibodies, such as FS118, are expected to bind to primed LAG-3-positive T cells in the lymph nodes which then migrate to the tumour site, carrying the bispecific antibody with them. Once within the tumour microenvironment, T cells carrying the bispecific antibody are expected to be able to engage and block PD-L1 on tumour cells. Alternatively, primed LAG-3-positive lymphocytes may have already infiltrated the tumour microenvironment (so-called “tumour infiltrating lymphocytes” or “TILs”). Thus, anti-LAG-3/PD-L1 bispecific antibodies, such as FS118, may bind to primed LAG-3-positive TILs (e.g. T cells) directly within the tumour microenvironment. T cells bound by anti-LAG-3/PD-L1 bispecific antibodies are thus expected to be resistant to both LAG-3 and PD-L1/PD-1 signalling, thereby preventing induction/maintenance of T cell exhaustion via these immune checkpoint proteins.

Similarly, PD-L1 expression is significantly increased in tumours and anti-LAG-3/PD-L1 bispecific antibodies, such as FS118, may therefore first localise to and concentrate in the tumour microenvironment through binding to PD-L1. The anti-LAG-3 portion can then bind to LAG-3 expressed on the surface of T cells present in the tumour microenvironment and prevent LAG-3-mediated suppression of the T cells.

Maintaining or prolonging the contact between T cells and tumour cells using anti-LAG-3/PD-L1 bispecific antibodies, such as FS118, increases the time in which the T cells can successfully recognise tumour antigens, become activated and proceed with killing the tumour cell, relative to combinations of individual monoclonal antibodies to these targets.

FS118 does not cross-react with and/or is not functional with respect to mouse LAG-3 or PD-L1. A mouse anti-LAG-3/PD-L1 (mLAG-3/mPD-L1; FS18m-108-29/S1 with LALA mutation) bispecific antibody capable of acting as a surrogate for FS118 in mouse experiments has been described. In syngeneic mouse models of cancer, the mLAG-3/mPD-L1 bispecific antibody was shown to be capable of enhanced or similar tumour growth suppression compared with the combined administration of two antibody molecules comprising the same LAG-3 and PD-L1 binding sites, respectively, when three doses of the antibody/antibodies were administered three days apart. The anti-mLAG-3/mPD-L1 antibody was also shown to be capable of preventing tumour growth in seven out of nine mice, whereas combined administration of two antibody molecules comprising the same LAG-3 and PD-L1 binding sites did not prevent tumour growth in any of the animals tested (WO2017/220569; P2399 A LAG-3/PD-L1 mAb² can overcome PD-L1-mediated compensatory upregulation of LAG-3 induced by single-agent checkpoint blockade, Faroudi et al., American Association for Cancer Research (AACR) Annual Meeting 2019, 29 Mar.-3 Apr. 2019, Atlanta, Ga., USA).

In addition to its tumour inhibition activity, there are early indications that the FS118 surrogate mLAG-3/mPD-L1 bispecific antibody exhibits a dose-response in a mouse tumour model, with higher doses (1 mg/kg to 20 mg/kg) generally correlating with reduced tumour volumes. The mLAG-3/mPD-L1 bispecific antibody has also been shown to induce LAG-3 suppression on LAG-3-expressing tumour infiltrating lymphocytes (TILs), whereas LAG-3 expression was increased when mice were treated with two antibody molecules comprising the same mLAG-3 and mPD-L1 binding sites as surrogate mLAG-3/mPD-L1 bispecific antibody. Both the surrogate mLAG-3/mPD-L1 bispecific antibody and the single agent combination have been shown to increase soluble LAG-3 and PD-L1 levels in the serum of treated mice (P348 Dual blockade of PD-L1 and LAG-3 with FS118, a unique bispecific antibody, induces T-cell activation with the potential to drive potent anti-tumour immune responses, Journal for ImmunoTherapy of Cancer 20175 (Suppl 2): 87; Abstract 2719: Dual blockade of PD-L1 and LAG-3 with FS118, a unique bispecific antibody, induces CD8+ T-cell activation and modulates the tumour microenvironment to promote antitumour immune responses, Cancer Research, July 2018 Volume 78, Issue 13 Supplement; P2399 A LAG-3/PD-L1 mAb² can overcome PD-L1-mediated compensatory upregulation of LAG-3 induced by single-agent checkpoint blockade, Faroudi et al., American Association for Cancer Research (AACR) Annual Meeting 2019, 29 Mar.-3 Apr. 2019, Atlanta, Ga., USA).

However, whilst the data in relation to FS118 surrogate mLAG-3/mPD-L1 bispecific antibody strongly indicates that the FS118 molecule per se will be therapeutically efficacious in humans, the mouse model used suffers from drawbacks in relation to predicting specific therapeutic doses for use in humans. In particular, the surrogate mLAG-3/mPD-L1 bispecific antibody has a human IgG1 backbone which will naturally elicit a strong immunogenic response in mice and the production of anti-drug antibodies (ADAs). Thus, the skilled person would not reasonably expect that effective doses used in mice would be effective in NHPs and, ultimately, humans.

Data on the behaviour of LAG-3/PD-L1 antibodies, including FS118, in human patients or non-human primates, including dosages for administration, have not been available to date.

### STATEMENTS OF INVENTION

FS118 is a bispecific antibody which binds to both LAG-3 and PD-L1, and which is expected to mediate its anti-tumour effect in a unique manner compared with monospecific anti-PD-L1 and LAG-3 antibodies as explained in the Background section above. In view of the bispecific, tetravalent nature of FS118 and the resulting differences in the stoichiometry of binding compared with monospecific, bivalent antibodies, as well as the expected differences in the mechanism of action of FS118, it was unclear whether FS118 could be dosed using dose levels and administration schedules used for monospecific anti-PD-L1 and anti-LAG3 antibodies in humans.

Anti-PD-L1 antibodies approved for cancer treatment in human patients, such as avelumab, durvalumab and atezolizumab are administered to cancer patients at a doses of 800 mg (flat dose) or 10 mg/kg (once every two weeks), 10 mg/kg (once every two weeks) and 1200 mg (once every three weeks) (equating to around 12 mg/kg in a standard 100 kg patient), respectively. A combination of the anti-LAG3 monoclonal antibody relatlimab and the anti-PD1 monoclonal antibody nivolumab is currently being tested in a Phase I clinical trial and is administered once every four weeks. Relatlimab treatment alone has also been evaluated in a phase I study where the antibody was dosed every 2 weeks.

A mouse LAG-3/PD-L1 (mLAG-3/mPD-L1; FS18m-108-29/S1 with LALA mutation) bispecific antibody capable of acting as a surrogate for FS118 in mouse experiments showed superior, or similar, anti-tumour efficacy in a syngeneic mouse tumour model as two monospecific antibody molecules comprising the same mLAG-3 and mPD-L1 binding sites as the mLAG-3/mPD-L1 bispecific antibody, when the antibodies were administered at the same dosage levels (1 mg/kg, 3 mg/kg and 10 mg/kg) and according to the same dosage schedule (3 doses, 3 days apart). However, the surrogate mLAG-3/mPD-L1 bispecific antibody has a human IgG1 backbone which will naturally elicit a strong immunogenic response in mice and the production of anti-drug antibodies (ADAs). Thus, it was not possible to predict whether or not the effective doses used in mice would be effective in non-human primates (NHPs) and, ultimately, humans.

When the PK of the mLAG-3/mPD-L1 bispecific antibody was evaluated in mice (at 1, 3, 10 and 20 mg/kg), the present inventors surprisingly found that the mLAG-3/mPD-L1 bispecific antibody was cleared from serum at a higher rate than a monospecific antibody comprising the same mPD-L1 binding site as the mLAG-3/mPD-L1 antibody. The non-saturable clearance of the mLAG-3/mPD-L1 bispecific antibody was further shown to appear to be a consequence of the combination of mPD-L1 binding and the target-specific changes of the permissive residues in the CH3 domain as compared against a control anti-mPD-L1 mAb. (Example 1).

By combining the mouse PK data obtained by the inventors with the anti-tumour efficacy data in mice, the present inventors found that exposure (Cₘₐₓ) to the mouse surrogate mAb² of ≥6 μg/mL was required for anti-tumour efficacy in mice and that this level of exposure surprisingly did not need to be maintained throughout the dosing period. However, ADA formation did appear to be occurring (Example 1).

In cynomolgus monkeys, a single dose (4 mg/kg) PK study, a non-good laboratory practice (GLP) dose range finding (DRF) study (once weekly iv doses of 10, 50 and 200 mg/kg for 4 wks) and repeated twice weekly iv administration in a 4 wk GLP toxicity study (60 and 200 mg/kg) found that FS118 was cleared faster than a monospecific anti-hPD-L1 mAb (Example 1).

Maintenance of FS118 plasma levels of ≥10 μg/ml throughout the dosing period was found to be sufficient to maintain PD-L1 capture, and by inference PD-L1 suppression and immune pharmacology in cynomolgus monkeys. These studies also showed that FS118 was well-tolerated even at high doses and indicated that high doses would also be well-tolerated in humans. As in mice, ADA formation was also observed (Example 1).

The results obtained from the mouse and cynomolgus monkey PK studies thus unexpectedly demonstrated that despite the rate of clearance of FS118 and FS18m-108-29AA/S1 relative to respective monospecific anti-PD-L1 antibodies, the very low antibody C_{trough} levels observed between doses were nevertheless sufficient to provide a sustained anti-tumour and pharmacodynamic response, respectively. Nevertheless, ADA formation in mice and cynomolgus monkeys was observed, indicating that these animal models did have limitations in terms of extrapolating from the observed results to humans.

A Phase I dose escalation and cohort expansion first-in-human study of the safety, tolerability, pharmacokinetics, and activity of FS118 in patients (study subjects) with advanced malignancies that have progressed on or after prior anti-PD-1/PD-L1 therapy was then designed and commenced. To assess safety, single patient cohorts were administered 800 μg, 2400 μg, 0.1 mg/kg, 0.3 mg/kg, and 1.0 mg/kg doses of FS118. For the dose-escalation part of the Phase I study, patients were administered 3 mg/kg, 10 mg/kg, and 20 mg/kg of FS118. All doses were administered once weekly (i.e. once per week), and therefore less frequently than was initially thought necessary based on the mouse and cynomolgus monkey PK data alone (Examples 1 and 2).

The interim results from 24 subjects initially (increasing to 43 patients) of the Phase I study confirmed that the maximum observed concentration (Cₘₐₓ) was in line with the Cₘₐₓ predicted from the cynomolgus monkey study but, surprisingly, that the rate of clearance of FS118 was higher than predicted, with AUC (area under the concentration versus time curve) being 30% lower than expected. This might initially suggest that higher doses of FS118 in humans would be needed. However, despite the rate of clearance being faster than originally predicted, longer term pharmacodynamic effects were observed, indicative of therapeutic efficacy (Example 2).

In particular, FS118 was shown to induce a sustained increase in soluble LAG-3 (sLAG-3) levels at doses of 3 mg/kg, 10 mg/kg and 20 mg/kg administered once weekly, as well as sustained LAG-3 receptor occupancy. sLAG-3, through its binding to MHCII, has been reported to stimulate antigen presenting cells such as macrophages and dendritic cells to activate T cell responses and enhance tumour-specific cytotoxic T cells, and is expected to thereby potentiate the anti-tumour immune response. In addition, sLAG3 levels had previously been shown to be associated with tumour growth suppression in mice, indicating that increased sLAG3 levels are indicative of therapeutic efficacy. Early results also suggested that sPD-L1 levels were also increased following FS118 treatment (Example 2).

The interim results from the first 24 subjects recruited in the Phase I study (increasing to 43 patients) thus surprisingly demonstrated that:
(i) administration of FS118 to human cancer patients at doses of 3 mg/kg to 20 mg/kg once weekly resulted in a sustained pharmacodynamic response which is expected to correlate with anti-tumour efficacy despite a faster rate of clearance than predicted of FS118 from patient serum, and
(ii) FS118 exposure throughout the dosing interval was not needed for a pharmacodynamic effect in human patients.

In addition, the initial results of the ongoing Phase I study have provided early direct evidence of efficacy of FS118 in the treatment of cancer (despite this not being a primary objective of the study). More specifically, by May 2019, 5 of the 14 patients for whom at least 1 “on-study” scan has been reported showed some stable disease. By August 2019, this had increased to 11 of 22 patients and by April 2020 to 17 of 30 patients (Example 2).

Data from the Phase I study were analysed to guide dose selection for future trials (Example 6). Bayesian analysis of the Phase I best overall response (BOR/iBOR) data estimated that there is a greater likelihood of patients exhibiting stable disease as BOR/iBOR if receiving 10 mg/kg or 20 mg/kg of FS118 once weekly than 3 mg/kg FS118 once weekly. Patients receiving 3 mg/kg FS118 once weekly also had higher levels of anti-drug antibodies compared with patients receiving 10 mg/kg or 20 mg/kg FS118 once weekly. Dosing FS118 at 10 mg/kg to 20 mg/kg once weekly is therefore preferred from the perspective of minimising potential immunogenicity and toxicity. Pharmacokinetic/Pharmacodynamic modelling and simulations of trimeric complex formation further showed that trimeric LAG3:FS118:PD-L1 complex concentration is expected to be highest at a dose of 10 mg/kg FS118 once weekly, assuming a biodistribution coefficient of 10%. Higher trimeric complex formation is hypothesized to translate into T cell activation and inhibition of tumour growth. Although patients receiving dosages as low as 3 mg/kg once weekly have shown stable disease (Table 8), administration of 10 mg/kg to 20 mg/kg of FS118 once weekly is preferred on the basis of increased efficacy and an expectation of reduced toxicity and immunogenicity. Dosages at the lower end of this range, such as 10 mg/kg of FS118 once weekly, are particularly preferred, as lower doses are thought to reduce the risk of T cell overstimulation and thus T cell exhaustion, thereby increasing the likelihood of a sustained therapeutic effect, as well as reducing the cost of treatment.

The FS118 antibody comprises the heavy chain sequence set forth in SEQ ID NO: 1 and the light chain sequence set forth in SEQ ID NO: 2.

Thus, in one aspect, the present invention provides an antibody molecule which binds PD-L1 and LAG-3 for use in a method of treating cancer in a human patient,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of at least 3 mg per kg of body weight of the patient.

In another aspect, the present invention provides a method of treating cancer in a human patient, wherein the method comprises administering to the patient a therapeutically effective amount of an antibody molecule which binds PD-L1 and LAG-3,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of at least 3 mg per kg of body weight of the patient.

In a further aspect, the present invention provides the use of antibody molecule which binds PD-L1 and LAG-3 in the manufacture of a medicament for treating cancer in a human patient,
wherein the antibody molecule comprises the heavy chain sequence set forth in SEQ ID NO: 1 and the light chain sequence set forth in SEQ ID NO: 2; and
wherein the treatment comprises administering the antibody molecule to the patient once weekly at a dose of at least 3 mg per kg of body weight of the patient.

FS118 may be administered to the patient at a dose of at least 4 mg per kg of body weight of the patient (mg/kg), at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, at least 15 mg/kg, at least 16 mg/kg, at least 17 mg/kg, at least 18 mg/kg, at least 19 mg/kg, or at least 20 mg/kg. In a preferred embodiment, FS118 is administered to the patient at a dose of at least 10 mg/kg. In an alternative preferred embodiment, FS118 is administered to the patient at a dose of at least 20 mg/kg. Other doses, such as administration of FS118 at a dose of at least 1 mg/kg are also contemplated.

In addition, or alternatively, FS118 may be administered at a dose of up to 10 mg/kg, up to 11 mg/kg, up to 12 mg/kg, up to 13 mg/kg, up to 14 mg/kg, up to 15 mg/kg, up to 16 mg/kg, up to 17 mg/kg, up to 18 mg/kg, up to 19 mg/kg, or up to 20 mg/kg. In a preferred embodiment, FS118 is administered at a dose of up to 10 mg/kg. In an alternative preferred embodiment, FS118 is administered at a dose of up to 20 mg/kg.

Thus, FS118 may be administered at a dose of 1 mg/kg to 20 mg/kg, 3 mg/kg to 20 mg/kg, or 10 mg/kg to 20 mg/kg. Alternatively, FS118 may be administered at a dose of 1 mg/kg to 10 mg/kg, or 3 mg/kg to 10 mg/kg. In a preferred embodiment, FS118 is administered at a dose of 3 mg/kg to 20 mg/kg, more preferably at a dose of 10 mg/kg to 20 mg/kg.

In one embodiment, FS118 is administered to the patient at a dose of 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg. For example, FS118 may be administered to the patient at a dose of 3 mg/kg. In a preferred embodiment, FS118 is administered to the patient at a dose of 10 mg/kg. In an alternative preferred embodiment, FS118 is administered to the patient at a dose of 20 mg/kg.

FS118 may be administered to the patient at a dose calculated based on the patient's weight in kilograms (kg) as described above. A patient receiving a dose of 10 mg/kg and weighing 70 kg, would thus receive a dose of 700 mg of FS118. Alternatively, FS118 may be administered to the patient at a flat dose, i.e. a dose which is not based on the patient's individual weight. A suitable flat dose for FS118 can be calculated based on the average weight of patients in a patient population, such as 70 kg, 75 kg, 80 kg, 85 kg, 90 kg, 95 kg, or 100 kg. In a preferred embodiment, the flat dose for FS118 is calculated based on 70 kg as the average patient weight. In an alternative preferred embodiment, the flat dose for FS118 is calculated based on 80 kg as the average patient weight. In a further preferred embodiment, the flat dose for FS118 is calculated based on 100 kg as the average patient weight.

Assuming an average patient weight of 100 kg, the present invention thus provides:

An antibody molecule which binds PD-L1 and LAG-3 for use in a method of treating cancer in a human patient,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of at least 300 mg.

A method of treating cancer in a human patient, wherein the method comprises administering to the patient a therapeutically effective amount of an antibody molecule which binds PD-L1 and LAG-3,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of at least 300 mg.

The use of antibody molecule which binds PD-L1 and LAG-3 in the manufacture of a medicament for treating cancer in a human patient,
wherein the antibody molecule comprises the heavy chain sequence set forth in SEQ ID NO: 1 and the light chain sequence set forth in SEQ ID NO: 2; and
wherein the treatment comprises administering the antibody molecule to the patient once weekly at a dose of at least 300 mg.

Assuming an average patient weight of 100 kg, FS118 may alternatively be administered to the patient at a dose of at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, or at least 2000 mg. For example, FS118 may be administered to the patient at a dose of at least 300 mg. In a preferred embodiment, FS118 is administered to the patient at a dose of at least 1000 mg. In an alternative preferred embodiment, FS118 is administered to the patient at a dose of at least 2000 mg. Other doses, such as administration of FS118 at a dose of at least 100 mg are also contemplated. In addition, or alternatively, FS118 may be administered at a dose of up to 1000 mg, 1100 mg, 1200 mg, 1300 mg, 1400 mg, 1500 mg, 1600 mg, 1700 mg, 1800 mg, 1900 mg, or 2000 mg, assuming an average patient weight of 100 kg. In a preferred embodiment, FS118 is administered at a dose of up to 1000 mg. In an alternative preferred embodiment, FS118 is administered at a dose of up to 2000 mg.

Thus, FS118 may be administered at a dose of 100 mg to 2000 mg, 300 mg to 2000 mg, or 1000 mg to 2000 mg, assuming an average patient weight of 100 kg. Alternatively, FS118 may be administered at a dose of 100 mg to 1000 mg, or 300 mg to 1000 mg. In a preferred embodiment, FS118 is administered at a dose of 300 mg to 2000 mg, more preferably at a dose of 1000 mg to 2000 mg.

For example, FS118 may be administered to the patient at a dose of 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg, assuming an average patient weight of 100 kg. For example, FS118 may be administered to the patient at a dose of 300 mg. In a preferred embodiment, FS118 is administered to the patient at a dose of 1000 mg. In an alternative preferred embodiment, FS118 is administered to the patient at a dose of 2000 mg.

Alternative flat doses, and flat dose ranges, for FS118 can be calculated using an alternative average weight of a patient population, such as 70 kg, 75 kg, 80 kg, 85 kg, 90 kg, or 95 kg, in particular, 70 kg or 80 kg, and administered to human cancer patients in accordance with the present invention.

For example, assuming an average patient weight of 70 kg, the present invention provides:

An antibody molecule which binds PD-L1 and LAG-3 for use in a method of treating cancer in a human patient,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of at least 210 mg.

A method of treating cancer in a human patient, wherein the method comprises administering to the patient a therapeutically effective amount of an antibody molecule which binds PD-L1 and LAG-3,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of at least 210 mg.

The use of antibody molecule which binds PD-L1 and LAG-3 in the manufacture of a medicament for treating cancer in a human patient,
wherein the antibody molecule comprises the heavy chain sequence set forth in SEQ ID NO: 1 and the light chain sequence set forth in SEQ ID NO: 2; and
wherein the treatment comprises administering the antibody molecule to the patient once weekly at a dose of at least 210 mg.

Assuming an average patient weight of 70 kg, FS118 may alternatively be administered to the patient at a dose of at least 280 mg, at least 350 mg, at least 420 mg, at least 490 mg, at least 560 mg, at least 630 mg, at least 700 mg, at least 770 mg, at least 840 mg, at least 910 mg, at least 980 mg, at least 1050 mg, at least 1120 mg, at least 1190 mg, at least 1260 mg, at least 1330 mg, or at least 1400 mg. For example, FS118 may be administered to the patient at a dose of at least 210 mg. In a preferred embodiment, FS118 is administered to the patient at a dose of at least 700 mg. In an alternative preferred embodiment, FS118 is administered to the patient at a dose of at least 1400 mg.

In addition, or alternatively, FS118 may be administered at a dose of up to 700 mg, 770 mg, 840 mg, 910 mg, 980 mg, 1050 mg, 1120 mg, 1190 mg, 1260 mg, 1330 mg, or 1400 mg, assuming an average patient weight of 70 kg. In a preferred embodiment, FS118 is administered at a dose of up to 700 mg. In an alternative preferred embodiment, FS118 is administered at a dose of up to 1400 mg. Other doses, such as administration of FS118 at a dose of at least 70 mg are also contemplated.

Thus, FS118 may be administered at a dose of 70 mg to 1400 mg, 210 mg to 1400 mg, or 700 mg to 1400 mg, assuming an average patient weight of 70 kg. Alternatively, FS118 may be administered at a dose of 70 mg to 700 mg, or 210 mg to 700 mg. In a preferred embodiment, FS118 is administered at a dose of 210 mg to 1400 mg, more preferably at a dose of 700 mg to 1400 mg.

For example, FS118 may be administered to the patient at a dose of 210 mg, 280 mg, 350 mg, 420 mg, 490 mg, 560 mg, 630 mg, 700 mg, 770 mg, 840 mg, 910 mg, 980 mg, 1050 mg, 1120 mg, 1190 mg, 1260 mg, 1330 mg, or 1400 mg, assuming an average patient weight of 70 kg. For example, FS118 may be administered to the patient at a dose of 210 mg. In a preferred embodiment, FS118 is administered to the patient at a dose of 700 mg. In an alternative preferred embodiment, FS118 is administered to the patient at a dose of 1400 mg.

As a further alternative, FS118 may be administered to the patient at a dose sufficient to achieve a mean trough plasma concentration (C_{trough}) of at least 0.1-10 μg/mL between doses. Without wishing to be bound by theory, these C_{trough} levels correlate with the EC₅₀ of FS118 in a human primary cell functional assay in vitro and thus may represent the pharmacologically active levels of FS118.

A mean trough plasma concentration plasma concentration of at least 10 μg/mL is expected to provide continuous inhibition of PD-L1.

Where FS118 is administered to the patient once weekly, the doses of FS118 may be separated in time by 7 or 8 days. As will be appreciated in the art, the time between doses may be varied to some extent so that each and every dose is not separated by precisely the same time. This will often be directed under the discretion of the administering physician. Thus, the doses of FS118 may be separated in time by a clinically acceptable range of time, such as from about 7 or 8 days.

FS118 may be administered to patients in three-week treatment cycles.

FS118 is preferably administered to the patient by intravenous injection.

A cancer to be treated in accordance with the present invention has preferably been subjected to prior treatment with one or more immune checkpoint inhibitors other than FS118.

A cancer to be treated in accordance with the present invention (i) may be refractive to, (ii) may have relapsed during or following, or (iii) may be responsive to treatment with one or more immune checkpoint inhibitors. In a preferred embodiment, the cancer to be treated in accordance with the present invention has relapsed during or following, prior treatment with one or more immune checkpoint inhibitors (other than FS118). The immune checkpoint inhibitor is preferably a PD-1 or PD-L1 inhibitor, more preferably an anti-PD-1 or anti-PD-L1 antibody. The prior treatment with one or more immune checkpoint inhibitors (other than FS118) may have been administered alone or in combination with one or more additional therapies (e.g. one or more chemotherapeutic agents).

The present inventors have surprisingly identified a subgroup of cancer patients that are more likely to experience longevity of disease control, i.e. sustained disease control, as a result of FS118 treatment. The patients in this subgroup are patients with tumours that showed a partial response to a prior anti-PD-1 or anti-PD-L1 therapy, or showed stable disease for more than 3 months whilst subjected to a prior anti-PD-1 or anti-PD-L1 therapy. These tumours are therefore considered to have an “acquired resistance phenotype” to the prior anti-PD-1 or anti-PD-L1 therapy. Patients which showed a complete response to an anti-PD-1 or anti-PD-L1 therapy are also expected to fall within this subgroup. Whether a tumour shows a complete response, partial response, stable disease or progressive disease during treatment with an anti-cancer therapy, such as an anti-PD-1 or anti-PD-L1 therapy may be evaluated according to the RECIST 1.1 criteria (Eisenhauer, 2009) or the iRECIST criteria (Seymour, 2017), preferably the RECIST 1.1 criteria. This may involve obtaining scans (e.g. MRI scans) of the patient's tumour and measuring the size/volume of the tumour lesions. For the purposes of defining acquired resistance herein, it is assumed that, where a patient had, for example, a first scan classified as showing stable disease (or partial response or complete response) followed by a later scan classified as showing progressive disease, the patient showed stable disease (or a partial response or complete response) for the time period until which the scan showing progressive disease was obtained. In other words, the acquired resistance phenotype may be defined as tumours that (a) had a best overall response (BOR) of complete response or partial response to a prior anti-PD-1 or anti-PD-L1 therapy, or (b) had stable disease as a best overall response (BOR) and were treated for more than 3 months with the anti-PD-1 or anti-PD-L1 therapy. Clinical endpoints such as BOR may be defined according to the RECIST 1.1 criteria (Eisenhauer, 2009) or the iRECIST criteria (Seymour, 2017), preferably the RECIST 1.1 criteria.

In contrast, patients with tumours which showed stable disease for 3 months or less (thus including tumours which showed no stable disease and therefore showed progressive disease from the beginning of treatment) whilst subjected to a prior anti-PD-1 or anti-PD-L1 therapy (in other words, tumours which had a BOR of stable disease and were treated for 3 months or less, including tumours which had a BOR of progressive disease) did not experience longevity of disease control and these tumours are therefore considered to have a “primary resistance phenotype” to the prior anti-PD-1 or anti-PD-L1 therapy. A patient with a tumour having an acquired resistance phenotype to a prior anti-PD-1 or anti-PD-L1 therapy may be referred to as having acquired resistance to the anti-PD-1 or anti-PD-L1 therapy. Similarly, a patient with a tumour having a primary resistance phenotype to a prior anti-PD-1 or anti-PD-L1 therapy may be referred to as having primary resistance to the anti-PD-1 or anti-PD-L1 therapy. The prior anti-PD-1 or anti-PD-L1 therapy may have been administered alone or in combination with one or more additional therapies (e.g. one or more chemotherapeutic agents and/or immunotherapeutic agents).

Specifically, all of the patients completing 18 weeks or more on FS118 treatment were shown to have tumours with an acquired resistance phenotype to a prior anti-PD-1 or anti-PD-L1 therapy, with the exception of one patient for whom the BOR was unknown (FIGS. 7 and 8). However, it was known for this latter patient with unknown BOR that they had stayed on the prior anti-PD-1 therapy for more than one year and thus it is suspected that this patient would have had a BOR that would classify as having acquired resistance. None of the patients with tumours with a primary resistance phenotype to a prior anti-PD-1 or anti-PD-L1 therapy received more than 17 weeks of FS118 treatment in the Phase I study (FIGS. 7 and 8). The increased likelihood of enhanced longevity of response to FS118 therapy in patients with tumours with an acquired resistance phenotype to a prior anti-PD-1 or anti-PD-L1 therapy was observed independently of the dose of FS118 administered and tumour type (FIGS. 7 to 9). Thus, a tumour's resistance status to prior anti-PD-1 or anti-PD-L1 therapy is indicative of the probability of sustained response to FS118 therapy. Specifically, a tumour with an acquired resistance phenotype to a prior anti-PD-1 or anti-PD-L1 therapy has a higher likelihood of responding to treatment with FS118, in particular responding to FS118 therapy for 18 weeks or more, 19 weeks or more, or 20 weeks or more, but preferably 18 weeks or more, than a tumour with a primary resistance phenotype to a prior anti-PD-1 or anti-PD-L1 therapy. Response to treatment with FS118 thus preferably refers to the tumour exhibiting stable disease, a partial response or a complete response to FS118 treatment, e.g. for 18 weeks or more, 19 weeks or more, or 20 weeks or more, but preferably 18 weeks or more.

The above finding of the inventors is particularly significant because re-treatment of patients with a PD-(L)1 antibody after disease progression on a prior PD-(L)1 containing treatment regimens is not recommended and historically patients have been shown to derive little benefit from such therapy (Fujita et al., Anticancer Res. 2019; Fujita et al., Thoracic Cancer, 2019; Martini et al., J. Immunotherapy Cancer, 2017).

Thus, in a further aspect, the present invention provides an antibody molecule which binds PD-L1 and LAG-3 for use in a method of treating cancer in a human patient who has been subjected to treatment with a prior anti-PD-1 or anti-PD-L1 therapy, the antibody molecule comprising the heavy chain sequence set forth in SEQ ID NO: 1 and the light chain sequence set forth in SEQ ID NO: 2;
wherein a tumour of the patient has been determined to have an acquired resistance phenotype in respect of the prior anti-PD-1 or anti-PD-L1 therapy, and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

A tumour, as referred to herein, may be a tumour lesion.

The present invention also provides an antibody molecule which binds PD-L1 and LAG-3 for use in a method of treating cancer in a human patient who has been subjected to treatment with a prior anti-PD-1 or anti-PD-L1 therapy, the antibody molecule comprising the heavy chain sequence set forth in SEQ ID NO: 1 and the light chain sequence set forth in SEQ ID NO: 2;
wherein the method comprises determining whether a tumour of the patient has an acquired resistance phenotype in respect of the anti-PD-1 or anti-PD-L1 therapy, wherein
a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy, and
treating a tumour determined to have acquired resistance phenotype to the prior anti-PD-1 or anti-PD-L1 therapy with the antibody.

Also provided is a method of treating cancer in a human patient who has been subjected to treatment with a prior anti-PD-1 or anti-PD-L1 therapy, the method comprising administering to the patient a therapeutically effective amount of an antibody molecule which binds PD-L1 and LAG-3 and comprises the heavy chain sequence set forth in SEQ ID NO: 1 and the light chain sequence set forth in SEQ ID NO: 2;
wherein a tumour of the patient has been determined to have acquired resistance phenotype in respect of the prior anti-PD-1 or anti-PD-L1 therapy, and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

Further provided is a method of treating cancer in a human patient who has been subjected to treatment with a prior anti-PD-1 or anti-PD-L1 therapy, the method comprising administering to the patient a therapeutically effective amount of an antibody molecule which binds PD-L1 and LAG-3 and comprises the heavy chain sequence set forth in SEQ ID NO: 1 and the light chain sequence set forth in SEQ ID NO: 2;
wherein the method comprises determining whether a tumour of the patient has an acquired resistance phenotype in respect of the prior anti-PD-1 or anti-PD-L1 therapy, and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy, and
treating a tumour determined to have acquired resistance phenotype to the prior anti-PD-1 or anti-PD-L1 therapy with the antibody.

In a further embodiment, the present invention provides the use of antibody molecule which binds PD-L1 and LAG-3 and comprises the heavy chain sequence set forth in SEQ ID NO: 1 and the light chain sequence set forth in SEQ ID NO: 2 in the manufacture of a medicament for treating cancer in a human patient who has been subjected to treatment with a prior anti-PD-1 or anti-PD-L1 therapy,
wherein a tumour of the patient has been determined to have an acquired resistance phenotype in respect of the prior anti-PD-1 or anti-PD-L1 therapy, and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

In a yet further embodiment, the present invention provides a method of determining whether a cancer patient who has been subjected to treatment with a prior anti-PD-1 or anti-PD-L1 therapy is likely to respond to treatment with an antibody molecule which binds PD-L1 and LAG-3 and comprises the heavy chain sequence set forth in SEQ ID NO: 1 and the light chain sequence set forth in SEQ ID NO: 2,
the method comprising determining whether a tumour of the patient has an acquired resistance phenotype, or primary resistance phenotype, in respect of the prior anti-PD-1 or anti-PD-L1 therapy,
wherein a tumour with an acquired resistance phenotype has a higher likelihood of responding to treatment with the antibody than a tumour with a primary resistance phenotype; and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy,
and a tumour with a primary resistance phenotype is a tumour which achieved stable disease for 3 months or less whilst subjected to treatment with the prior anti-PD-1 or anti-PD-L1 therapy, including a tumour with a best overall response of progressive disease.

A likelihood of response preferably refers to the likelihood that the tumour will exhibit stable disease, a partial response or a complete response to treatment with FS118, e.g. for 18 weeks or more, 19 weeks or more, or 20 weeks or more, but preferably 18 weeks or more.

The present invention also provides a method of predicting the likelihood of response of a cancer patient to an antibody molecule which binds PD-L1 and LAG-3 and comprises the heavy chain sequence set forth in SEQ ID NO: 1 and the light chain sequence set forth in SEQ ID NO: 2,
wherein the patient is predicted to be likely to respond to the antibody if a tumour of the patient has been determined to have an acquired resistance phenotype in respect of a prior anti-PD-1 or anti-PD-L1 therapy,
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

In another embodiment, the present invention provides a method of selecting a patient who has been subjected to treatment with a prior anti-PD-1 or anti-PD-L1 therapy, for treatment with an antibody molecule which binds PD-L1 and LAG-3 and comprises the heavy chain sequence set forth in SEQ ID NO: 1 and the light chain sequence set forth in SEQ ID NO: 2,
the method comprising determining whether a tumour of the patient has an acquired resistance phenotype, or primary resistance phenotype, in respect of the prior anti-PD-1 or anti-PD-L1 therapy,
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy, and
a tumour with a primary resistance phenotype is a tumour which achieved stable disease for 3 months or less whilst subjected to treatment with the prior anti-PD-1 or anti-PD-L1 therapy, including a tumour with a best overall response of progressive disease; and
selecting a patient with a tumour determined to have an acquired resistance phenotype for treatment with the antibody.

Anti-PD-1 or anti-PD-L1 therapy may refer to treatment with an anti-PD-1 or anti-PD-L1 antibody (other than an antibody which binds to both PD-L1 and LAG-3, such as FS118), including, but not limited to, treatment with nivolumab, pembrolizumab, avelumab, durvalumab or atezolizumab.

The present inventors have further shown that the percentage of tumour cells that showed positive staining for PD-L1 prior to treatment with FS118 in tumours with an acquired resistance phenotype positively correlated with longevity of disease control as a result of FS118 treatment. In the acquired resistance group, the three patients treated with FS118 for 30 weeks or more also had the highest percentage of tumour cells which showed positive staining for PD-L1 at baseline. No such correlation was seen in patients with primary resistance to anti-PD-1 or anti-PD-L1 therapy (FIG. 10). These results show that tumours with an acquired resistance phenotype to prior anti-PD-1 or anti-PD-L1 therapy, which comprise 15% or more, 20% or more, or 25% or more, but preferably 15% or more, PD-L1 positive tumour cells are more likely to respond to treatment with FS118. For example, tumours with an acquired resistance phenotype to prior anti-PD-1 or anti-PD-L1 therapy may comprise 15% or more, 16% or more, 17% or more, 18% or more, or 19% or more PD-L1 positive tumour cells.

Methods for determining the percentage of PD-L1 positive tumour cells in a tumour sample are known in the art and may comprise staining of a tumour sample with an anti-PD-L1 antibody and detecting binding of the antibody to the tumour cells either directly or indirectly. The percentage of PD-L1 positive tumour cells can be determined by counting the number tumour cells, e.g. in 5 high power fields, and determining the percentage of said tumour cells to which the antibody is bound.

In a further embodiment, the present invention thus provides an antibody molecule which binds PD-L1 and LAG-3 for use in a method of treating cancer in a human patient who has been subjected to treatment with a prior anti-PD-1 or anti-PD-L1 therapy, the antibody molecule comprising the heavy chain sequence set forth in SEQ ID NO: 1 and the light chain sequence set forth in SEQ ID NO: 2;
wherein a tumour of the patient has been determined to have an acquired resistance phenotype in respect of the prior anti-PD-1 or anti-PD-L1 therapy, and a sample of the tumour obtained from the patient prior to treatment with the antibody has been determined to comprise 15% or more PD-L1 positive tumour cells,
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

The present invention also provides an antibody molecule which binds PD-L1 and LAG-3 for use in a method of treating cancer in a human patient who has been subjected to treatment with a prior anti-PD-1 or anti-PD-L1 therapy, the antibody molecule comprising the heavy chain sequence set forth in SEQ ID NO: 1 and the light chain sequence set forth in SEQ ID NO: 2; and
wherein the method comprises determining whether:
(i) a tumour of the patient has an acquired resistance phenotype to the prior anti-PD-1 or anti-PD-L1 therapy; and
(ii) a sample of the tumour obtained from the patient prior to treatment with the antibody comprises 15% or more PD-L1 positive tumour cells; and
treating a tumour determined to have acquired resistance phenotype and comprising 15% or more PD-L1 positive tumour cells, with the antibody;
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

Also provided is a method of treating cancer in a human patient who has been subjected to treatment with a prior anti-PD-1 or anti-PD-L1 therapy, the method comprising administering to the patient a therapeutically effective amount of an antibody molecule which binds PD-L1 and LAG-3 and comprises the heavy chain sequence set forth in SEQ ID NO: 1 and the light chain sequence set forth in SEQ ID NO: 2;
wherein a tumour of the patient has been determined to have acquired resistance phenotype to in respect of the prior anti-PD-1 or anti-PD-L1 therapy, and a sample of the tumour obtained from the patient prior to treatment with the antibody has been determined to comprise 15% or more PD-L1 positive tumour cells;
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

Further provided is a method of treating cancer in a human patient who has been subjected to treatment with a prior anti-PD-1 or anti-PD-L1 therapy, the method comprising administering to the patient a therapeutically effective amount of an antibody molecule which binds PD-L1 and LAG-3 and comprises the heavy chain sequence set forth in SEQ ID NO: 1 and the light chain sequence set forth in SEQ ID NO: 2;
wherein the method comprises determining whether:
(i) a tumour of the patient has an acquired resistance phenotype in respect of the prior anti-PD-1 or anti-PD-L1 therapy; and
(ii) a sample of the tumour obtained from the patient prior to treatment with the antibody comprises 15% or more PD-L1 positive tumour cells; and
treating a tumour determined to have acquired resistance phenotype and comprising 15% or more PD-L1 positive tumour cells, with the antibody;
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

In a further embodiment, the present invention provides the use of antibody molecule which binds PD-L1 and LAG-3 and comprises the heavy chain sequence set forth in SEQ ID NO: 1 and the light chain sequence set forth in SEQ ID NO: 2 in the manufacture of a medicament for treating cancer in a human patient who has been subjected to treatment with a prior anti-PD-1 or anti-PD-L1 therapy,
wherein a tumour of the patient has been determined to have acquired resistance phenotype in respect of the prior anti-PD-1 or anti-PD-L1 therapy, and a sample of the tumour obtained from the patient prior to treatment with the antibody has been determined to comprise 15% or more PD-L1 positive tumour cells;
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

In a yet further embodiment, the present invention provides a method of determining whether a cancer patient who has been subjected to treatment with a prior anti-PD-1 or anti-PD-L1 therapy is likely to respond to treatment with an antibody molecule which binds PD-L1 and LAG-3 and comprises the heavy chain sequence set forth in SEQ ID NO: 1 and the light chain sequence set forth in SEQ ID NO: 2,
the method comprising determining whether:
(i) a tumour of the patient has acquired resistance phenotype or primary resistance phenotype in respect of the prior anti-PD-1 or anti-PD-L1 therapy; and
(ii) a sample of the tumour sample obtained from the patient prior to treatment with the antibody comprises 15% or more PD-L1 positive tumour cells;
wherein a tumour with an acquired resistance phenotype comprising at least 15% PD-L1 positive tumour cells has a higher likelihood of responding to treatment with the antibody than a tumour with a primary resistance phenotype, or a tumour with an acquired resistance phenotype comprising less than 15% PD-L1 positive tumour cells;
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy, and
a tumour with a primary resistance phenotype is a tumour which achieved stable disease for 3 months or less whilst subjected to treatment with the prior anti-PD-1 or anti-PD-L1 therapy, including a tumour with a best overall response of progressive disease. The method may further comprise selecting a tumour determined to have acquired resistance phenotype to a prior anti-PD-1 or anti-PD-L1 therapy and comprising 15% or more PD-L1 positive tumour cells for treatment, or treating a tumour determined to have and acquired resistance phenotype to a prior anti-PD-1 or anti-PD-L1 therapy and having a cancer comprising 15% or more PD-L1 positive tumour cells, with the antibody.

The present invention also provides a method of predicting the likelihood of response of a cancer patient to an antibody molecule which binds PD-L1 and LAG-3 and comprises the heavy chain sequence set forth in SEQ ID NO: 1 and the light chain sequence set forth in SEQ ID NO: 2,
wherein the patient is predicted to be likely to respond to the antibody if a tumour of the patient has been determined to have an acquired resistance phenotype in respect of a prior anti-PD-1 or anti-PD-L1 therapy, and a sample of the tumour obtained from the patient prior to treatment with the antibody has been determined to comprise 15% or more PD-L1 positive tumour cells,
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

In another embodiment, the present invention provides a method of selecting a patient who has been subjected to treatment with a prior anti-PD-1 or anti-PD-L1 therapy, for treatment with an antibody molecule which binds PD-L1 and LAG-3 and comprises the heavy chain sequence set forth in SEQ ID NO: 1 and the light chain sequence set forth in SEQ ID NO: 2,
the method comprising determining whether:
(i) a tumour of the patient has an acquired resistance phenotype or primary resistance phenotype in respect of the prior anti-PD-1 or anti-PD-L1 therapy; and
(ii) a sample of the tumour obtained from the patient prior to treatment with the antibody comprises 15% or more PD-L1 positive tumour cells; and
selecting a patient with a tumour determined to have acquired resistance phenotype and comprising 15% or more PD-L1 positive tumour cells, for treatment with the antibody,
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy, and
a tumour with a primary resistance phenotype is a tumour which achieved stable disease for 3 months or less whilst subjected to treatment with the prior anti-PD-1 or anti-PD-L1 therapy, including a tumour with a best overall response of progressive disease.

In the above aspects of the invention and embodiments, the antibody may be administered to the patient at a dose, according to a dosing schedule, and/or route of administration as disclosed herein.

In a particularly preferred embodiment, the present invention thus provides an antibody molecule which binds PD-L1 and LAG-3 for use in a method of treating cancer, preferably squamous cell carcinoma of the head and neck (SCCHN), in a human patient who has been subjected to treatment with a prior anti-PD-1 or anti-PD-L1 therapy, the antibody molecule comprising the heavy chain sequence set forth in SEQ ID NO: 1 and the light chain sequence set forth in SEQ ID NO: 2, wherein the method comprises administering the antibody molecule to the patient once weekly at a dose of 10 mg per kg of body weight of the patient, and
wherein a tumour of the patient has been determined to have an acquired resistance phenotype in respect of the prior anti-PD-1 or anti-PD-L1 therapy,
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

In a further preferred embodiment, the present invention also provides a method of treating cancer, preferably SCCHN, in a human patient who has been subjected to treatment with a prior anti-PD-1 or anti-PD-L1 therapy, the method comprising administering to the patient a therapeutically effective amount of an antibody molecule which binds PD-L1 and LAG-3 comprising the heavy chain sequence set forth in SEQ ID NO: 1 and the light chain sequence set forth in SEQ ID NO: 2, wherein the method comprises administering the antibody molecule to the patient once weekly at a dose of 10 mg per kg of body weight of the patient, and
wherein a tumour of the patient has been determined to have an acquired resistance phenotype in respect of the prior anti-PD-1 or anti-PD-L1 therapy,
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

### BRIEF DESCRIPTION OF THE FIGURES

FIG. 1 shows the effect on T cell LAG-3 expression following treatment of tumours with anti-PD-1/PD-L1 monotherapy (left-hand panel), a combination of anti-PD-1/PD-L1 and anti-LAG-3 monotherapies (central panel), and the bispecific anti-PD-1/PD-L1 antibody FS118 (right-hand panel).

FIG. 2 shows the expected effect of FS118 treatment on tumours that are refractive to or have relapsed following anti-PD-1/PD-L1 therapy, and tumours which are responsive to anti-PD-1/PD-L1 therapy.

FIG. 3A shows the mean (±SEM) tumour volume after administration of 10 mg/kg test article (200 μg per mouse) on days 3, 6 and 9 post tumour implantation. FS118 mouse surrogate mAb²=mLAG-3/PD-L1; FS18m-108-29AA/4420=mLAG-3/mock mAb²; PD-L1 BM1 mAb=anti-PD-L1 mAb; mLAG-3 BM1 mAb=anti-LAG-3 mAb; IgG control=G1AA/4420. B shows PK data—single dose i.v. administration 10 mg/kg for mLAG-3/PD-L1 (open circles and triangles) and anti-PD-L1 mAb (filled circles and triangles). Data from two different studies, represented by circles and triangles, respectively is shown.

FIG. 4 shows tumour volume measurements in the MC38 syngeneic tumour model grown subcutaneously in C57BL/6 mice treated with 3 doses of G1AA/4420 (IgG control, 10 mg/kg) and the anti-mouse LAG-3/PD-L1 mAb² FS18m-108-29AA/S1 at 4 different doses (1 mg/kg, 3 mg/kg, 10 mg/kg, and 20 mg/kg). Each dose is indicated by a vertical black arrow on the x-axis. The mean tumour volume plus or minus standard error mean (SEM) is shown on the y-axis. Comparison of tumour size on Study Day 17 was made between isotype control group (G1AA/4420) and LAG-3/PD-L1 mAb² treated groups using One-Way Analysis of Variance (ANOVA). Significant differences between groups were determined using Tukey's Multiple Comparisons Test: ***P≤0.001; ****P≤0.0001.

FIG. 5 shows the structure of the 2-compartment population PK model describing the systemic exposure to FS118 constructed from the NHP non-GLP and GLP PK data (0-7 days post-dose). V_{P} (which may also be referred to as V1)=Central Volume; V_{T} (which may also be referred to as V2)=Peripheral Volume; CL_{d} (which may also be referred to as CL2)=Exchange Coefficient; CL_{P} (which may also be referred to as CL1)=FS118 clearance; C_{P}=Plasma Compartment; C_{T}=Tissue Compartment.

FIG. 6 shows the first-in-human (FIH) study design for the Phase I study in Example 2.

FIG. 7 shows weeks of FS118 treatment completed as of 25 Mar. 2020 in relation to resistance group and dose (diamonds=1 mg/kg; circles=3 mg/kg; triangles=10 mg/kg; squares=20 mg/kg). A significant difference was observed between patients with acquired resistance to anti-PD-1/PD-L1 therapy, as defined herein, as compared with patients with primary resistance to anti-PD-1/PD-L1 therapy, as defined herein, wherein patients with Acquired resistance remain on FS118 treatment for longer on average than patients with Primary resistance regardless of the FS118 dose administered.

FIG. 8 shows a swimmer plot for 39 patients categorised as having primary or acquired resistance to anti-PD-1/PD-L1 therapy (ordered by the FS118 dose) who had evaluable tumour scans as of 27 Nov. 2019 whilst receiving FS118 treatment. Patients with primary resistance are shown as grey bars, while patients with acquired resistance are shown as dark grey bars. The number of weeks of FS118 treatment completed is shown (PD=progressive disease; SD=stable disease). All patients with more than 18 weeks of FS118 treatment completed (all bar one of whom had tumours with an acquired resistance phenotype) had at least one measurement of stable disease.

FIG. 9 shows weeks of FS118 treatment completed as of 25 Mar. 2020 based on the same data as presented in FIG. 7, but in relation to resistance group and tumour type. Likelihood of response to FS118 treatment was linked to tumours with an acquired resistance phenotype but appears to be independent of clinical indication (tumour type).

FIG. 10 shows the percentage of tumour cells in tumour biopsy samples showing positive staining for PD-L1 (PD-L1 percent tumour positive score [PD-L1% TPS]) prior to FS118 treatment in relation to number of weeks of FS118 treatment completed as of 12 Dec. 2019. A: A high baseline PD-L1% TPS showed a positive correlation with length of FS118 treatment for patients with acquired resistance to PD-1/PD-L1 therapy. The three patients with the highest PD-L1% TPS within the acquired resistance group were treated with FS118 for 30 weeks or more, evidencing disease control by FS118. B: No correlation between PD-L1% TPS and length of FS118 treatment was observed for patients with primary resistance to anti-PD-1/PD-L1 therapy.

FIG. 11 shows that patients with acquired resistance to anti-PD-1/PD-L1 therapy showed a higher magnitude immune cell response with FS118 treatment than patients with primary resistance to anti-PD-1/PD-L1 therapy. The percentage change of immune cell counts over time (open circle: CD3+ T cells, filled square: CD4+ T cells, filled triangle: CD8+ T cells, filled diamond: NK cells) is depicted as percentage change from baseline before the start of FS118 treatment. A: Patient 1004-0003 is a representative patient profile with primary resistance. B: patient 1002-0014 is a representative patient profile with acquired resistance. Data shown obtained 26 Nov. 2019.

### DETAILED DESCRIPTION

### Anti-LAG-3/PD-L1 Bispecific Antibodies

Anti-LAG-3/PD-L1 bispecific antibodies (such as FS118 described herein), suitable for use in the present invention are described in WO2017/220569 A1, the contents of which are incorporated herein in their entirety and for all purposes. The FS118 antibody comprises the heavy chain sequence set forth in SEQ ID NO: 1 and the light chain sequence set forth in SEQ ID NO: 2.

### Cancer

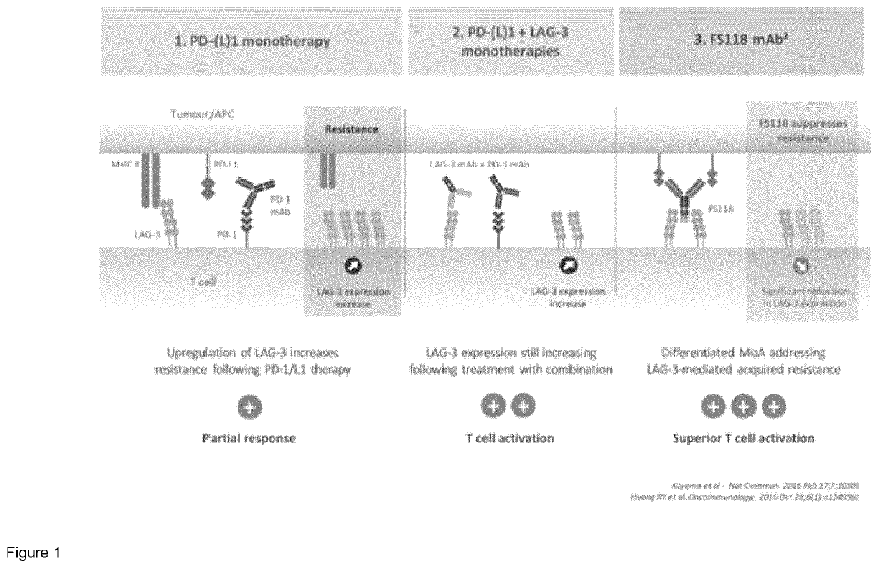

PD-1, its ligand PD-L1, and LAG-3 are examples of immune checkpoint proteins. Molecules such as antibodies which bind to and inhibit these proteins are collectively referred to as immune checkpoint inhibitors. Treatment of cancer patients with anti-PD-1/PD-L1 antibodies as monotherapy has been shown to result in up-regulation of LAG-3 expression on T cells, resulting in resistance to anti-PD-L1/PD-1 therapy (FIG. 1). Combined treatment with anti-PD-1/PD-L1 antibodies and anti-LAG-3 antibodies was not capable of preventing the increase in LAG-3 expression on T cells, although the increase in expression was reduced compared with anti-PD-L1/PD-1 therapy alone (FIG. 1). In contrast, treatment with FS118 (and the mouse surrogate antibody FS18m-108-29AA/S1) has been shown to result in reduced T cell LAG-3 expression, as well as increased sLAG-3 levels (FIG. 1). FS118 thus has a different mode of action compared with anti-PD-L1/PD-1 and anti-LAG-3 antibodies and is capable of preventing and/or reversing LAG-3-mediated resistance to PD-L1/PD-1 inhibitors, as demonstrated by the early results from the Phase I study, which showed a pharmacodynamic response, as well as stable disease in several patients, following FS118 treatment in patients with locally advanced, unresectable, or metastatic solid tumours or haematological malignancies that had progressed while on, or after, anti-PD-1/PD-L1 therapy.

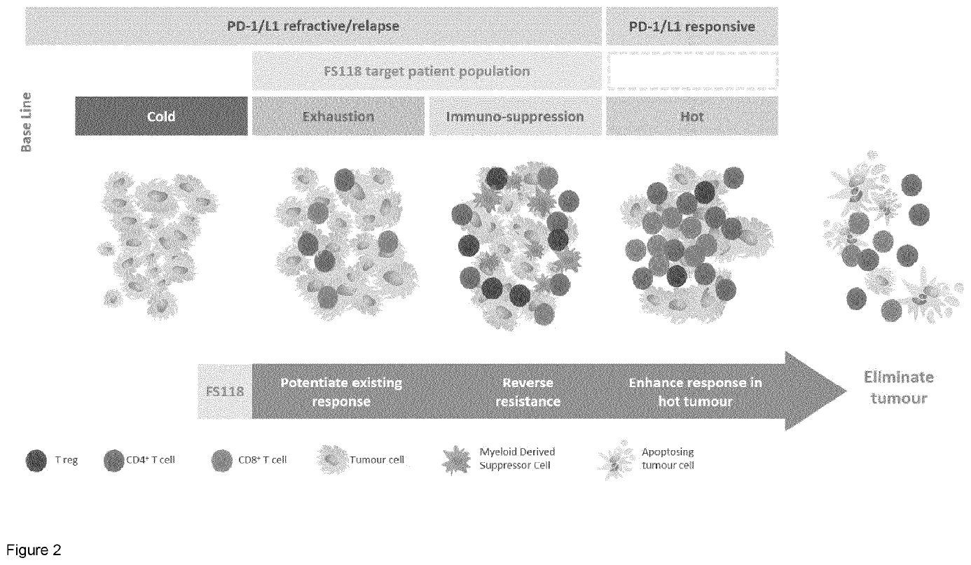

Without being bound by theory, the expected effect of FS118 treatment on tumours that are refractive to, or have relapsed during or following, anti-PD-1/PD-L1 monotherapy, and tumours which are responsive to anti-PD-1/PD-L1 monotherapy, is shown in FIG. 2.

A cancer which is refractive to treatment with one or more immune checkpoint inhibitors, preferably refers to a cancer which is resistant to treatment with one or more immune checkpoint inhibitors (other than a LAG-3/PD-L1 bispecific antibody, such as FS118). A cancer which has relapsed during or following treatment with one or more immune checkpoint inhibitors, preferably refers to cancer which has acquired resistance to one or more immune checkpoint inhibitors (other than a LAG-3/PD-L1 bispecific antibody, such as FS118) during or following treatment with said immune checkpoint inhibitor(s).

Specifically, FIG. 2 shows that in tumours that are refractive to, or have relapsed during or following, anti-PD-1/PD-L1 monotherapy and exhibit T cell exhaustion or immune-suppression, FS118 treatment is expected to potentiate an immune-mediated anti-cancer effect by reversing T cell exhaustion/immune-suppression as a consequence of binding to LAG-3 expressed on the T cell surface (which otherwise acts as an inhibitory signal to the immune cells), reducing T cell surface overexpression of LAG-3 and promoting the release of soluble LAG-3 (sLAG-3). FS118 thus has the potential to significantly broaden the clinical benefit of immune checkpoint blockade since it has the capability to rescue patients with primary or adaptive resistance to “standard-of-care” immune checkpoint inhibitor therapy.

In tumours which are responsive to PD-1/PD-L1 monotherapy, it is expected that TILs express LAG-3 on their surface and tumours are PD-L1 high. By binding to said LAG-3 and PD-L1, FS118 is expected to enhance T-cell activation in these patients over and above anti-PD-1/PD-L1 monotherapy, as well as preventing overexpression of LAG-3 in response to anti-PD-L1 treatment. Thus, development of resistance to PD-L1 blockade is expected to be suppressed. Dosages for FS118, as disclosed herein, which have been shown to result in a pharmacodynamic response, as well as stable disease in several patients with tumours or haematological malignancies that had progressed while on, or after, anti-PD-1/PD-L1 therapy, are therefore also expected to be suitable to effectively treat cancers which are responsive to PD-1/PD-L1 monotherapy.

Cancers which show response to immune checkpoint inhibitor treatment must comprise TILs to mediate said effect. Thus, cancers which are refractive to or have relapsed during or following treatment with an immune checkpoint inhibitor other than an anti-PD-1/PD-L1 inhibitor are expected to comprise inactive TILs (i.e. exhausted or immuno-suppressed), whilst cancers which are responsive to treatment with immune checkpoint inhibitors other than anti-PD-1/PD-L1 inhibitors are expected to comprise activated TILs. As a consequence, it is expected that FS118 will have a similar effect on PD-L1 expressing cancers which are refractive to or have relapsed during or following treatment with an immune checkpoint inhibitor other than an anti-PD-1/PD-L1 inhibitor, or are responsive to treatment with an immune checkpoint inhibitor other than an anti-PD-1/PD-L1 inhibitor, as described for cancers which are refractive to or have relapsed, or are responsive to, treatment with anti-PD-1/PD-L1 inhibitors above.

In a preferred embodiment, a cancer to be treated in accordance with the present invention has therefore been subjected to prior treatment with one or more immune checkpoint inhibitors (other than a LAG-3/PD-L1 bispecific antibody, such as FS118).

A cancer to be treated in accordance with the present invention may therefore be, or have been determined to be, refractive to treatment with one or more immune checkpoint inhibitors (other than a LAG-3/PD-L1 bispecific antibody, such as FS118). Alternatively, a cancer to be treated in accordance with the present invention may have relapsed during or following treatment with one or more immune checkpoint inhibitors (other than a LAG-3/PD-L1 bispecific antibody, such as FS118). As a further alternative, a cancer to be treated in accordance with the presence invention may be responsive to, or have been determined to be responsive to, treatment with one or more immune checkpoint inhibitors. Relapse of a cancer during or following treatment with one or more immune checkpoint inhibitors preferably refers to cancer progression during or following treatment with one or more immune checkpoint inhibitors. Detection of cancer progression is well within the capabilities of the skilled person.

The immune checkpoint inhibitor may be a PD-1, PDL-1, PD-L2, CTLA-4, CD80, CD86, LAG-3, B7-H3, VISTA, B7-H4, B7-H5, B7-H6, NKp30, NKG2A, Galectin 9, TIM-3, HVEM, BTLA, KIR, CD47, or SiRP alpha inhibitor. The immune checkpoint inhibitor may be an antibody capable of inhibiting the immune checkpoint molecule in questions. In a preferred embodiment, the immune checkpoint inhibitor is a PD-1 or PD-L1 inhibitor, such as an anti-PD1/PD-L1 antibody. Antibodies capable of inhibiting immune checkpoint molecules are known in the art and include ipilimumab for inhibition of CTLA-4; nivolumab, pembrolizumab, and cemiplimab for PD-1; and atezolizumab, avelumab, and durvalumab for PD-L1. Immune checkpoint molecules, their ligands and inhibitors are reviewed in Marin-Acevedo et al. Journal of Hematology & Oncology (2018).

A cancer to be treated in accordance with the present invention expresses PD-L1. Preferably, the cancer has been determined to express PD-L1. In addition, a cancer to be treated in accordance with the present invention comprises LAG-3 expressing immune cells, such as TILs. Preferably, the cancer has been determined to comprise LAG-3 expressing immune cells. In a preferred embodiment, the cancer may be a cancer which is resistant to treatment with one or more immune checkpoint inhibitors (other than a LAG-3/PD-L1 bispecific antibody, such as FS118) due to expression of PD-L1 by the cancer cells and LAG-3 expression on the surface of immune cells. In particular embodiments, the expression of PD-L1 on the surface of cancer cells and expression of LAG-3 on the surface of immune cells within the tumour microenvironment may be high, relative to normal tissue cells and activated immune cells respectively.

The present inventors have surprisingly shown that tumours which have an acquired resistance phenotype to a prior anti-PD-1 or anti-PD-L1 therapy, and in particular have an acquired resistance phenotype to a prior anti-PD-1 or anti-PD-L1 therapy and comprise at least 15% PD-L1-positive tumour cells prior to treatment with FS118, have an increased likelihood of showing a sustained response, in particular sustained stable disease, in response to treatment with FS118. This effect was observed independently of tumour type and FS118 dosage administered.

A cancer to be treated in accordance with the present invention thus preferably has an acquired resistance phenotype to an anti-PD-1 or anti-PD-L1 therapy, as defined herein. Yet more preferably, a cancer to be treated in accordance with the present invention has an acquired resistance phenotype to an anti-PD-1 or anti-PD-L1 therapy, as defined herein, and a tumour(s) of the cancer comprise(s) at least 15% PD-L1-positive tumour cells prior to treatment with FS118.

A cancer to be treated using an antibody molecule of the invention may be selected from the group consisting of head and neck cancer (such as squamous cell carcinoma of the head and neck (SCCHN)), Hodgkin's lymphoma, non-Hodgkin's lymphoma (such as diffuse large B-cell lymphoma, indolent non-Hodgkin's lymphoma, mantle cell lymphoma, ovarian cancer, prostate cancer, colorectal cancer, fibrosarcoma, renal cell carcinoma, melanoma, pancreatic cancer, breast cancer, glioblastoma multiforme, lung cancer (such as non-small cell lung cancer or small cell lung cancer), stomach cancer (gastric cancer), bladder cancer, cervical cancer, uterine cancer, vulvar cancer, testicular germ cell cancer, penile cancer, leukemia (such as chronic lymphocytic leukemia, myeloid leukemia, acute lymphoblastoid leukaemia, or chronic lymphoblastoid leukaemia), multiple myeloma, squamous cell cancer, testicular cancer, esophageal cancer (such as adenocarcinoma of the esophagogastric junction), Kaposi's sarcoma, and central nervous system (CNS) lymphoma, hepatocellular carcinoma, nasopharyngeal cancer, Merkel cell carcinoma, mesothelioma, thyroid cancer (such as anaplastic thyroid cancer), and sarcoma (such as soft tissue sarcoma). Tumours of these cancers are known, or expected, to express PD-L1 on their cell surface and/or contain immune cells, such as TILs, expressing PD-L1 and/or LAG-3.

Treatment of renal cell carcinoma, lung cancer (such as non-small cell lung cancer or small cell lung cancer), nasopharyngeal cancer, colorectal cancer, melanoma, stomach cancer (gastric cancer), esophageal cancer (such as adenocarcinoma of the esophagogastric junction), ovarian cancer, cervical cancer, bladder cancer, head and neck cancer (such as SCCHN), leukemia (such as chronic lymphocytic leukemia, Hodgkin's lymphoma, non-Hodgkin's lymphoma (such as diffuse large B-cell lymphoma, indolent non-Hodgkin's lymphoma, mantle cell lymphoma), and multiple myeloma using anti-LAG-3 antibodies has been investigated in clinical trials and shown promising results. Thus, the cancer to be treated using the antibody molecules of the present invention may be head and neck cancer (such as SCCHN), a renal cell carcinoma, lung cancer (such as non-small cell lung cancer or small cell lung cancer), nasopharyngeal cancer, colorectal cancer, melanoma, stomach cancer (gastric cancer), esophageal cancer (such as adenocarcinoma of the esophagogastric junction), ovarian cancer, cervical cancer, bladder cancer, leukemia (such as chronic lymphocytic leukemia, Hodgkin's lymphoma, non-Hodgkin's lymphoma (such as diffuse large B-cell lymphoma, indolent non-Hodgkin's lymphoma, mantle cell lymphoma), or multiple myeloma.

Treatment of melanoma, colorectal cancer, breast cancer, bladder cancer, renal cell carcinoma, bladder cancer, gastric cancer, head and neck cancer (such as SCCHN), mesothelioma, lung cancer (such as non-small-cell lung cancer or small cell lung cancer), ovarian cancer, Merkel-cell carcinoma, pancreatic cancer, melanoma and hepatocellular carcinoma using anti-PD-L1 antibodies has also been investigated in clinical trials and shown promising results. Thus, the cancer to be treated using the antibody molecules of the present invention may be head and neck cancer (such as SCCHN), a melanoma, colorectal cancer, breast cancer, bladder cancer, renal cell carcinoma, bladder cancer, gastric cancer, mesothelioma, lung cancer (such as non-small-cell lung cancer), ovarian cancer, Merkel-cell carcinoma, pancreatic cancer, melanoma, or hepatocellular carcinoma.

Preferred cancers for treatment using the antibody molecules of the present invention are head and neck cancer (such as SCCHN), lung cancer (such as non-small-cell lung cancer), bladder cancer, diffuse large B cell lymphoma, gastric cancer, pancreatic cancer and hepatocellular carcinoma. Tumours of these cancers are known to comprise LAG-3 expressing immune cells and to express PD-L1 either on their cell surface or to comprise immune cells expressing PD-L1.

In a preferred embodiment, the cancer is selected from the group consisting of: squamous cell carcinoma of the head and neck (SCCHN), gastric cancer, adenocarcinoma of the esophagogastric junction (GEJ), non-small cell lung cancer (NSCLC) (such as lung adenocarcinoma or lung squamous histological subtypes), melanoma (such as skin cutaneous melanoma), prostate cancer, bladder cancer (such as bladder urothelial carcinoma), breast cancer (such as triple negative breast cancer), colorectal cancer (CRC; for example, adenocarcinoma or the colon or rectum), renal cell carcinoma (RCC), hepatocellular carcinoma (HCC), small-cell lung cancer (SCLC) and Merkel cell carcinoma.

In an alternative preferred embodiment, the cancer is a rare cancer selected from the group consisting of: thyroid cancer (preferably anaplastic thyroid cancer), sarcoma (preferably soft tissue sarcoma), glioblastoma multiforme (GBM), sarcoma (e.g. soft tissue sarcoma including dedifferentiated lipsosarcoma, undifferentiated pleomorphic sarcoma and leiomyosarcoma), ovarian cancer (e.g. ovarian high/low-grade serous or clear cell histology), basal cell carcinoma, MSI-H solid tumours, triple negative breast cancer (TNBC), cervical cancer, oesophageal cancer (e.g. adenocarcinoma of the esophagogastric junction (GEJ) or squamous cell carcinoma of the oesophagus), multiple myeloma (MM), pancreatic cancer (such as pancreatic adenocarcinoma), meningioma, thyroid carcinoma, endometrial cancer (such as MSI-H endometrial cancer), thymic carcinoma, gestational trophoblastic neoplasia, lymphomas (such as diffuse large B-cell lymphoma (DLBCL), or peripheral T-cell lymphoma), peritoneal carcinomatosis, microsatellite stable (MSS) colorectal cancer and gastrointestinal stromal tumours (GIST) (such as unresectable GIST).

In one preferred embodiment, the cancer is thyroid cancer, preferably anaplastic thyroid cancer. In an alternative preferred embodiment, the cancer is sarcoma, preferably soft tissue sarcomas. It has recently been shown that the presence of tertiary lymphoid structures (TLS) within the sarcoma tumour tissue may predict response to immune checkpoint blockade therapies (Petitprez et al., 2020).

In another embodiment, the cancer to be treated may be selected from: head and neck cancer (such as SCCHN), gastric cancer, oesophageal cancer, NSCLC, mesothelioma, cervical cancer, thyroid cancer (such as anaplastic thyroid cancer) and sarcoma (such as soft-tissue sarcoma).

In one particular embodiment, the cancer to be treated is Head & Neck cancer, preferably squamous cell carcinoma of the head and neck (SCCHN), more preferably squamous cell carcinoma of the oral cavity, oropharynx, larynx or hypopharynx. The cancer may be relapsed or metastatic. Higher levels of co-expression of LAG-3 and PD-1 on T cells in the tumour microenvironment of SCCHN patients has been correlated with lack of responsiveness to anti-PD-1/PD-L1 agents (Hanna et al., 2018) and LAG-3 expression on TILs in SCCHN patients with negative lymph node status has been shown to be a prognostic marker of lower survival (Deng et al., 2016). Treatment with a bispecific antibody such as FS118 targeting both LAG-3 and PD-L1 simultaneously may reinvigorate an immune response as described herein. The Head & Neck cancer (such as SCCHN) may or may not have already been treated with, and progressed on, prior anti-PD-1 or anti-PD-L1 therapy (other than FS118) administered alone or in combination with another therapy (e.g. a chemotherapeutic agent). The patients may be positive or negative for Human papilloma virus (HPV). In one embodiment, the patients are all positive for HPV. In an alternative embodiment, the patients are all negative for HPV.

In another embodiment, the cancer to be treated is gastric cancer, which is known to express high levels of LAG-3 (Morgado et al., 2018). The gastric cancer may or may not have already been treated with, and progressed on, prior anti-PD-1 or anti-PD-L1 therapy (other than FS118) administered alone or in combination with another therapy (e.g. a chemotherapeutic agent). In a further embodiment, the cancer to be treated is NSCLC, preferably Stage IV squamous and/or Stage III NSCLC. The NSCLC may have already been treated with, and progressed on, prior anti-PD-1 or anti-PD-L1 therapy (other than FS118) administered alone or in combination with another therapy (e.g. a chemotherapeutic agent). In a further embodiment, the cancer to be treated is SCLC, preferably Extensive Stage SCLC. The SCLC may have already been treated with, and progressed on, prior anti-PD-1 or anti-PD-L1 therapy (other than FS118) administered alone or in combination with another therapy (e.g. a chemotherapeutic agent). In a yet further embodiment, the cancer to be treated is ovarian cancer. The ovarian cancer may be platinum-refractory and/or may or may not have been previously treated with an immunotherapy (e.g. an anti-PD-1 or anti-PD-L1 therapy (other than FS118) administered alone or in combination with another therapy (e.g. a chemotherapeutic agent)).

Where the application refers to a particular type of cancer, such as breast cancer, this refers to a malignant transformation of the relevant tissue, in this case a breast tissue. A cancer which originates from malignant transformation of a different tissue, e.g. ovarian tissue, may result in metastatic lesions in another location in the body, such as the breast, but is not thereby a breast cancer as referred to herein but an ovarian cancer.

A cancer to be treated in accordance with the present invention may be a primary cancer. Alternatively, the cancer may be a metastatic cancer.

### Route of Administration

FS118 is preferably administered to the patient by intravenous injection. For example, FS118 may be administered to the patient by intravenous bolus injection or intravenous infusion, e.g. using a continuous infusion pump. Intravenous infusion may be conducted using a continuous infusion pump over 30 minutes for doses of up to 2400 μg, and for doses above 2400 μg over 60 minutes. These administration types were successfully employed for FS118 in the Phase I study (Example 2).

### Formulations

For therapeutic use, the FS118 antibody is formulated with a carrier that is pharmaceutically acceptable and is appropriate for delivering the FS118 antibody by the chosen route of administration, such as intravenous administration. Suitable pharmaceutically acceptable carriers are those conventionally used for intravenous administration of antibody molecules, such as diluents and excipients and the like. Pharmaceutically acceptable carriers for therapeutic use are well known in the pharmaceutical art, and are described, for example, in Remington's Pharmaceutical Sciences, Mack Publishing Co. (A. R. Gennaro edit. 1985).

### Combination Treatments

A method of treating cancer as disclosed herein may comprise administration of the FS118 antibody to the patient either alone or in combination with other treatments. For example, the FS118 antibody may be administered concurrently, or sequentially, or as a combined preparation with another therapeutic agent or agents, dependent upon the cancer to be treated. For example, the FS118 antibody may be administered in combination with a known therapeutic agent for the cancer to be treated. For example, the FS118 antibody may be administered to the patient in combination with a second anti-cancer therapy, such as chemotherapy, anti-tumour vaccination (also referred to as a cancer vaccination), radiotherapy, immunotherapy, an oncolytic virus, chimeric antigen receptor (CAR) T-cell therapy, or hormone therapy.

It is expected that the FS118 antibody will act as an adjuvant in anti-cancer therapy, such as chemotherapy, anti-tumour vaccination, or radiotherapy. Without wishing to be bound by theory, it is thought that administration of the FS118 antibody to the patient in combination with chemotherapy, anti-tumour vaccination, or radiotherapy will trigger a greater immune response against the tumour-associated antigens, than is achieved with chemotherapy, anti-tumour vaccination, or radiotherapy alone.

A method of treating cancer in a patient may thus comprise administering to the patient a therapeutically effective amount of the FS118 antibody in combination with a chemotherapeutic agent, anti-tumour vaccine, radionuclide, immunotherapeutic agent, oncolytic viruses, CAR-T cells, or agent for hormone therapy. The chemotherapeutic agent, anti-tumour vaccine, radionuclide, immunotherapeutic agent, oncolytic viruses, CAR-T cells, or agent for hormone therapy is preferably a chemotherapeutic agent, anti-tumour vaccine, radionuclide, immunotherapeutic agent, oncolytic viruses, CAR-T cells, or agent for hormone therapy for the cancer in question, i.e. a chemotherapeutic agent, anti-tumour vaccine, radionuclide, immunotherapeutic agent, oncolytic viruses, CAR-T cells, or agent for hormone therapy which has been shown to be effective in the treatment of the cancer in question. The selection of a suitable chemotherapeutic agent, anti-tumour vaccine, radionuclide, immunotherapeutic agent, oncolytic viruses, CAR-T cells, or agent for hormone therapy which have been shown to be effective for the cancer in question is well within the capabilities of the skilled practitioner.

For example, where the method comprises administering to the patient a therapeutically effective amount of the FS118 antibody in combination with a chemotherapeutic agent, the chemotherapeutic agent may be selected from the group consisting of: taxanes, cytotoxic antibiotics, tyrosine kinase inhibitors, PARP inhibitors, B_RAF enzyme inhibitors, HDAC inhibitors, mTOR inhibitors, alkylating agents, platinum analogs, nucleoside analogs, thalidomide derivatives, antineoplastic chemotherapeutic agents and others. Taxanes include docetaxel, paclitaxel and nab-paclitaxel; cytotoxic antibiotics include actinomycin, bleomycin, anthracyclines, doxorubicin and valrubicin; tyrosine kinase inhibitors include erlotinib, gefitinib, osimertinib, afatinib, axitinib, PLX3397, imatinib, cobimitinib, trametinib, lenvatinib, cabozantinib, anlotinib, sorafenib, cediranib, regorafrinib, sitravatinib, pazopinib and defactinib; PARP inhibitors include niraparib, olaparib, rucaparib and veliparib; B-Raf enzyme inhibitors include vemurafenib and dabrafenib; alkylating agents include dacarbazine, cyclophosphamide, temozolomide; platinum analogs include carboplatin, cisplatin and oxaliplatin; nucleoside analogs include gemcitabine and azacitidine; antineoplastics include fludarabine. HDAC inhibitors include entinostat, panobinostat and varinostat; mTOR inhibitors include everolimus and sirolimus. Other chemotherapeutic agents suitable for use in the present invention include methotrexate, pemetrexed, capecitabine, eribulin, irinotecan, fluorouracil, and vinblastine.

Vaccination strategies for the treatment of cancers has been both implemented in the clinic and discussed in detail within scientific literature (such as Rosenberg, S. 2000 Development of Cancer Vaccines). This mainly involves strategies to prompt the immune system to respond to various cellular markers expressed by autologous or allogenic cancer cells by using those cells as a vaccination method, both with or without granulocyte-macrophage colony-stimulating factor (GM-CSF). GM-CSF provokes a strong response in antigen presentation and works particularly well when employed with said strategies.

Further aspects and embodiments of the invention will be apparent to those skilled in the art given the present disclosure including the following experimental exemplification.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Other aspects and embodiments of the invention provide the aspects and embodiments described above with the term “comprising” replaced by the term “consisting of” or “consisting essentially of”, unless the context dictates otherwise.

Certain aspects and embodiments of the invention will now be illustrated by way of example and with reference to the figures described above.

### EXAMPLES

### Example 1: First in Human (FIH) Dose Justification and Dose Escalation Strategy for FS118

FS118 is a bispecific antibody molecule which targets two immune checkpoint proteins, LAG-3 and PD-L1 simultaneously. FS118 has been shown to differ in a number of important respects from monospecific immune checkpoint inhibitors, such as anti-PD-L1 antibodies. These differences necessitated a detailed analysis to determine the appropriate dosages for a Phase I study of FS118 in human patients. Specifically, FS118 was tested in in vitro and in vivo studies to determine the optimal starting dose and dose escalation strategy for a Phase I human study designed to determine the safety, tolerability, pharmacokinetics, and activity of FS118 in patients with advanced malignancies that had progressed on or after prior PD-1/PD-L1 containing therapy (see Example 2 below).

1.1 FS118 and mLAG-3/PD-L1: Overview of Non-Clinical Studies

The non-clinical studies included PK studies in C57/BL6 wild-type wt mice, LAG-3 knock-out (KO) mice (see Example 1.3.1.1) and non-human primate (NHP; cynomolgus monkeys) with the clinical candidate FS118. The NHP studies included a single dose PK study (see Example 1.3.2.1), a Dose Range Finding toxicology study which included quantification of anti-drug antibodies (ADAs) and soluble PD-L1 (see Example 1.3.2.2) and a GLP (Good Laboratory Practice) toxicity study with similar quantification parameters (see Example 1.3.2.3).

With respect to studies in mice, FS118 has a reduced ability to bind to mLAG-3 and mPD-L1 compared with hLAG-3 and hPD-L1, respectively. Consequently, in vivo PK studies were also conducted with a surrogate mouse mAb² bispecific antibody (mLAG-3/PD-L1 [FS18-7-108-29/S1 with LALA mutation]) in C57/BL6 wt mice and LAG-3 knock-out mice on a C57/BL6 background (see Example 1.3.1.2). This mouse surrogate mAb² binds to the respective mouse target proteins with higher affinity compared with FS118. As part of the pharmacology studies, the mouse surrogate mAb² was also used in a mouse MC38 syngeneic tumour model and exposure data was collected at selected times during the dosing period to assess the PK and efficacy of the molecule (see Example 1.3.1.3).

The results of these studies fed into the development of an NHP PK model (see Example 1.5.1) and determination of the Highest Non-Severely Toxic Dose (HNSTD; see Example 1.5.2), which in turn led to the justification for the FIH starting dose (see Example 1.5.3).

1.2 Methods

1.2.1 Measurement of Serum/Plasma FS118 and Serum mLAG-3/PD-L1 in Mouse and NHP to Understand the Pharmacokinetics (PK) of FS118

In the mouse PK studies, serum FS118 and mLAG-3/PD-L1 were detected using a Mesoscale Discovery (MSD) human IgG kit according to the manufacturer's instruction (MSD Kit K150JLD-2); as such, the PK assay was expected to measure “total” FS118 i.e. regardless of any binding to either sLAG-3 or sPD-L1.

In the initial single dose NHP study (see Example 1.3.2.1), a customised electrochemiluminescence (ECL) Mesoscale Discovery (MSD) immunoassay was developed to detect serum FS118. Briefly, MSD 96-well plates (MSD #L15XB) were coated with an anti-human Fc mAb in order to capture FS118 (Abcam #ab124055) and blocked with MSD blocker A for 2 hours at room temperature. Serum samples were diluted 1:10 with MSD diluent and added to the wells and incubated for 2 hours at room temperature with shaking before the plates were washed 3 times with phosphate-buffered saline+0.05% Tween. To detect bound FS118, plates were then incubated with a sulfo-tagged anti-human IgG for 2 hours at room temperature, washed as in the previous step, and detected using 2×MSD read buffer. Readings were calibrated using a standard curve of 12 FS118 concentrations starting from 50 μg/mL.

Subsequent repeat dose studies in NHP (see Examples 1.3.2.2 and 1.3.2.3) employed customised LAG-3 capture/PD-L1 detection formats for the detection of plasma FS118. The preliminary DRF toxicology study was analysed with a qualified PK assay using an ECL MSD immunoassay. Standards and samples were added to the appropriate wells of an MSD microtitre plate pre-coated with recombinant human LAG-3 Fc chimera (R&D #2319-L3) as the capture reagent. Following a wash step, a biotinylated recombinant human PD-L1 Fc fusion protein (BPS, #71105) was added to each well and incubated. After a further wash step, a sulfo-tagged streptavidin detection reagent (MSD, #R32AD) was added and incubated. Following another wash step, MSD read solution was added and ECL was measured in order to detect the presence of FS118. For the good laboratory practice (GLP) toxicity study, plasma FS118 levels were measured using a validated Gyros immunoassay platform (Gyrolab) with biotinylated LAG-3 capture and Alexa Fluor® 647-labelled PD-L1 detection. Briefly, samples were diluted to 1:10 in Rexxip H buffer and added to plates which were then loaded onto the Gyrolab xP workstation. FS118 was detected by fluorescence emission. The standard curve was regressed using a 4 parameter logistic curve with response as the weighting factor (1/Y²) in the Gyrolab Evaluator software. The validated assay had a LLOQ of 39.1 ng/mL.

1.2.2 Measurement of Plasma Anti-FS118 Antibodies (ADA) in NHP

The presence of antibodies reactive to FS118 was measured in the NHP dose range finding (DRF) and 4 week good laboratory practice (4 wk GLP) toxicity studies (see Examples 1.3.2.2 and 1.3.2.3 respectively) using a standard electrochemical luminescence bridging format. To limit drug interference, biotinylated FS118 and sulfo-tagged FS118 were incubated with an acid-dissociated sample and labelled ADA complex was immobilised on a streptavidin plate before washing and subsequent detection. Assay sensitivity was 75 ng/mL for a polyclonal rabbit positive control anti-FS118 antibody and 150 ng/mL of the positive control could be detected in the presence of 96.5 μg/mL FS118.

1.2.3 Measurement of Plasma Total Soluble PD-L1 (sPD-L1)

The change in total sPD-L1 after administration of FS118 was quantified in the NHP DRF and 4 wk GLP toxicity studies (see Examples 1.3.2.2 and 1.3.2.3 respectively) using the Quantikine® Human/Cynomolgus Monkey B7-H1/PD-L1 Immunoassay (R&D Systems #DB7H10) according to the manufacturer's protocol. In this assay, FS118 interfered with the detection of Fc-labelled PD-L1 but did not appear to interfere with the detection of endogenous PD-L1 and the assay was therefore assumed to measure total PD-L1 (i.e. free PD-L1 and FS118-PD-L1 complex). The validated LLOQ for this assay was 25 μg/mL.

1.3 Pharmacokinetics and Pharmacodynamics in Animals

1.3.1 Mouse

1.3.1.1 FS118 PK in Wt and LAG-3 Knock-Out Mice

Although FS118 binds to mLAG-3 (with lower affinity compared with hLAG-3), it does not bind to mPD-L1 and has no functional activity against either target. FS118 had normal IgG kinetics in the C57/BL6 wt mouse, comparable to an isotype control antibody (Table 1). FS118 was also administered to LAG-3 KO mice and in these animals FS118 displayed normal IgG kinetics (Table 1).

TABLE 1
FS118 PK in wt and LAG-3 KO mice

| | C57/BL6 wt mouse | | | | | LAG-3 KO mouse | | | |
|---|---|---|---|---|---|---|---|---|---|
| | Dose | | Cmax | AUC(0-6) | | Dose | | Cmax | AUC(0-6) |
| Study | (mg/kg) | Route | (μg/mL) | (h · μg/mL) | Study | (mg/kg) | Route | (μg/mL) | (h · μg/mL) |
| MK033 | 10 | iv | 153 | 13,050 | MK028 | 10 | iv | 279 | 12,180 |
| | | | [99.9, 233] | | | | | [256, 324] | |
| | | | | | MK033 | 10 | iv | 154 | 11,420 |
| | | | | | | | | [95.1, 248] | |

AUC(0-6)—AUC 0-6 days after dosing (single dose)
Values are mean [95% CI]
n = up to 3 per dose group
Source data: [F-Star Study Report FS118_Pharm_015]

1.3.1.2 mLAG-3/PD-L1 mAb² PK in Wt and LAG-3 Knock-Out Mice

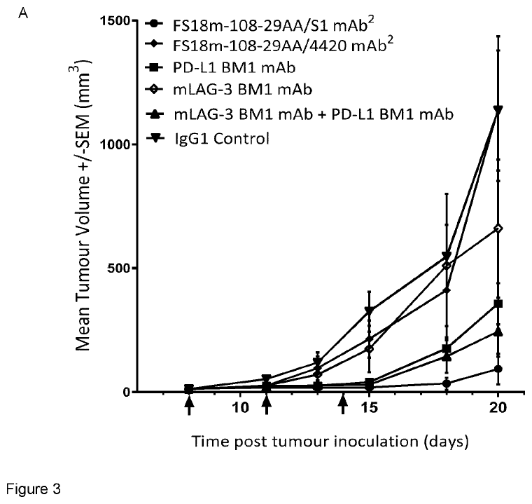

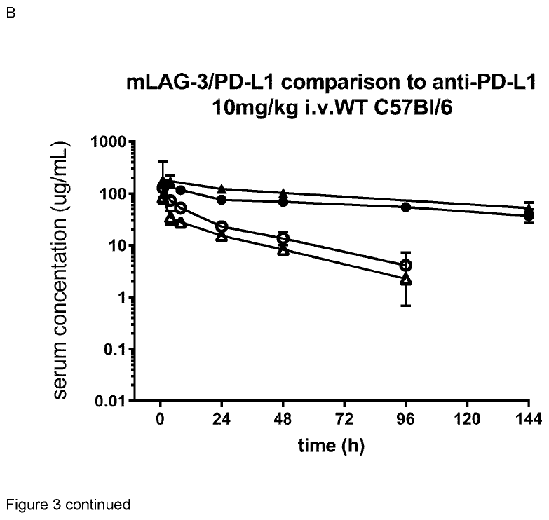

In contrast to FS118, the mouse surrogate mAb² (mLAG-3/PD-L1) was cleared from serum in the wt mouse more quickly (Table 2). The PK characteristics of the mouse surrogate mAb² were compared with an anti-PD-L1 mAb (same IgG1 framework, same Fab PD-L1 binding moiety) after single administration in wt mice. Despite the same PD-L1 binding epitope, the rate of clearance of the mAb² construct was higher than that of the mAb (FIG. 3B). Initially, this suggested that mLAG-3 target-binding may be responsible for the rate of clearance of the mouse surrogate mAb², since the anti-PD-L1 mAb did not exhibit the same clearance rate (FIG. 3B). However, in subsequent studies with a LAG-3 KO mouse, the mouse surrogate mAb² continued to display the clearance rate previously observed (Table 2), at first suggesting that this process could be most likely driven by PD-L1 binding in combination with the mAb² format. However, this phenomenon has not been observed with other mAb² indicating that the mAb² format as such is not responsible. Thus, the surrogate mAb² clearance is likely due to the combination of PD-L1 binding and the target-specific changes of the permissive residues in the CH3 domain as compared against the anti-PD-L1 mAb.

It should also be noted that despite the higher rate of clearance of the mouse surrogate mAb² compared with the anti-PD-L1 mAb, both constructs achieved a significant anti-tumour response in the MC38 syngeneic tumour model (FIG. 3A), demonstrating that the rate of clearance observed appears to be disconnected from longer term pharmacodynamic effects and does not preclude anti-tumour efficacy.

TABLE 2
mLAG-3/PD-L1 mAb² PK in wt and LAG-3 KO mice

| | C57/BL6 wt mouse | | | | | LAG-3 KO mouse | | | |
|---|---|---|---|---|---|---|---|---|---|
| | Dose | | Cmax | AUC(0-6) | | Dose | | Cmax | AUC(0-6) |
| Study | (mg/kg) | Route | (μg/mL) | (h · μg/mL) | Study | (mg/kg) | Route | (μg/mL) | (h · μg/mL) |
| MK033 | 10 | iv | 82.9 | 1,340 | MK028 | 10 | iv | 157 | 1,700 |
| | | | [61.1, 113] | | | | | [98.1, 250] | |
| | | | | | MK032 | 10 | iv | 92.8 | 1,150 |
| | | | | | | | | [91.8, 93.9] | |
| | | | | | MK033 | 10 | iv | 88.2 | 1,090 |
| | | | | | | | | [49.3, 158] | |

AUC(0-6)—AUC 0-6 days after dosing (single dose)
Values are mean [95% CI]
n = up to 3 per dose group
Source data: [F-Star Study Report FS118_Pharm_015]

1.3.1.3 mLAG-3/PD-L1 mAb² PK in an MC38 Tumour Model

Female C57/BL6 mice (The Jackson Laboratory (Street Bar Harbor, Me., USA)) aged 10-11 weeks and weighing 17.73-21.23 g (mean 19.49 g) were each inoculated into the subcutaneous space just below the animal's right shoulder with 1×10⁶ MC38 mouse colon carcinoma cells (National Cancer Institute (Bethesda, Md., USA)). Eight days post-inoculation (Study Day 0), sixty of the inoculated animals were randomised using a matched pair distribution method based on tumour size (mean tumour volume was approximately 55.6 mm³ (variability of 2.1%)) into six groups of 12, and treatment commenced. Animals in each group received intraperitoneal (i.p.) treatment with either FS18m-108-29AA/S1 (at doses of 0.40, 0.20, 0.06 or 0.02 mg/animal, equivalent to approximately 20, 10, 3 and 1 mg/kg), or negative control antibody G1AA/4420 (at 0.20 mg/animal, equivalent to approximately 10 mg/kg) in a fixed volume of 200 μL/animal. Three doses of each were administered (Study Days 0, 3 and 6). FS18m-108-29AA/S1 was diluted in formulation buffer and G1AA/4420 was diluted in Dulbecco's Phosphate Buffered Saline. All tumour measurements were acquired with the same hand-held callipers (Fowler Ultra-Cal V electronic calliper). Tumour dimensions (length and width) were measured for all animals on the first treatment day (Study Day 0) and then twice weekly (i.e. twice per week) until Study Day 53. Tumour volume was calculated using the equation: Tumour volume (mm³)=length×width²×π/6.

Serum samples were taken via terminal cardiac bleed one hour before the first dose and then at 71 h, 143 h, 148 h, 152 h, 168 h, 192 h, 240 h and 288 h after the first dose. Doses were administered at 0 h, 71 h and 143 h. Samples were stored at −80° C. and shipped on dry ice for analysis. Serum surrogate mAb² concentration was quantified per dose: C_{trough} and Cₘₐₓ levels and AUC are shown in Table 3. Trough concentrations prior to the second and the last dose decreased significantly, indicative of an ADA response.

TABLE 3
mLAG-3/PD-L1 mAb² PK in the MC38 tumour model

| | | | | | | | | D1 | D3 |
|---|---|---|---|---|---|---|---|---|---|
| | | Dose | | Ctrough (1) | Ctrough (2) | Ctrough (3) | Cmax | AUC(0-3) | AUC(0-3) |
| Study | Model | (mg/kg) | Route | (μg/mL) | (μg/mL) | (μg/mL) | (μg/mL) | (h · μg/mL) | (h · μg/mL) |
| MK029 | MC38 | 1 | ip | — | 0.1 | 0.2 | 3.41 | — | 47.5 |
| | | | | | [0.05, 0.23] | [0.05, 0.79] | [0.26, 44.6] | | |
| | | 3 | ip | 0.81 | 0.37 | 0.23 | 6.1 | — | 56.9 |
| | | | | [0.06, 10.6] | [0.23, 0.60] | [0.01, 5.60] | [1.18, 31.5] | | |
| | | 10 | ip | 2.91 | 0.49 | 0.25 | 5.46 | — | 44.9 |
| | | | | [0.60, 14.2] | [0.29, 0.86] | [0.41, 0.15] | [3.28, 9.08] | | |
| | | 20 | ip | 20.1 | 1.26 | 0.22 | 18.03 | — | 147.6 |
| | | | | [11.9, 33.9] | [0.51, 3.07] | [0.20, 0.24] | [12.6, 25.9] | | |

Ctrough (1)—trough concentration after dose 1, immediately before the second dose
Ctrough (2)—trough concentration after dose 2, immediately before the final dose
Ctrough (3)—trough concentration after dose 3, 72 h after the final dose
D3 Cmax—maximum observed conccentration after the last dose
D3 AUC(0-3)—AUC 0-3 days after the 3rd (i.e. last) dose
Source data: [F-Star Study Report F5118_Pharm_012]
Values are mean [95% CI]

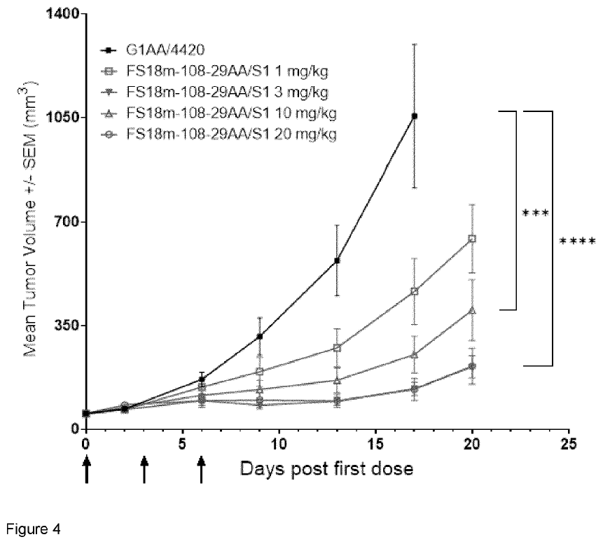

Despite this apparent decrease in exposure during the dosing period, noticeable tumour shrinkage was observed at all doses tested, with significant inhibition of the rate of tumour growth across the whole time of study observed at doses of 3, 10 and 20 mg/kg FS18m-108-29AA/S1 using a mixed model analysis (p≤0.05) compared with G1AA/4420 (FIG. 4). Comparison of tumour size on Study Day 17 specifically was made between isotype control group (G1AA/4420) and FS18m-108-29AA/S1 treated groups using One-Way Analysis of Variance (ANOVA); using Tukey's Multiple Comparisons Test, it was found that the tumour sizes after FS18m-108-29AA/S1 treatment at all dose groups (1, 3, 10 and 20 mg/kg) reduced significantly compared to the isotype control group (p<0.05). Kaplan-Meier survival analysis showed that FS18m-108-29AA/S1 mAb² induced a statistically significant survival benefit compared to isotype control in MC38 syngeneic model when dosed at 3, 10 or 20 mg/kg. Observed Cₘₐₓ and AUC (0-3 days) after the last dose were highly variable and it was not possible to make any definitive conclusion regarding dose-proportionality from this study.

Overall, these data suggest that exposure (Cₘₐₓ) to the mouse surrogate mAb² 6 μg/mL (Cₘₐₓ, last dose, 3 mg/kg) is required for anti-tumour efficacy and this level of exposure does not need to be maintained throughout the dosing period. In wt mice, mean Cₘₐₓ after a single 10 mg/kg dose of the mouse surrogate mAb² was 82.9 μg/mL (Table 2); equivalent to 25 μg/mL for a 3 mg/kg dose, assuming dose proportionality. Due to the possible impact of ADA formation, it is likely that this higher Cₘₐₓ was achieved after the first dose in the MC38 tumour bearing mice at the 3 mg/kg dose.

1.3.2 Non-Human Primate

The Pharmacokinetic-Pharmacodynamic behaviour of FS118 in NHP was characterized in three separate studies: intravenous administration of FS118 in (i) a single dose (4 mg/kg) PK study, (ii) a non-GLP DRF study (once weekly iv doses 10, 50 and 200 mg/kg for 4 wks), and (iii) twice weekly iv administration in a 4 wk GLP toxicity study (60 and 200 mg/kg).

The rate of clearance of FS118 was higher than was expected for a human IgG-like molecule in NHP and the rate of clearance did not exhibit typical “target-mediated” behaviour (i.e. saturation of target-mediated clearance at high exposure levels) at doses up to 200 mg/kg. Overall, in the NHP studies, there was an approximate dose proportional increase in Cₘₐₓ after the first dose across the dose range 4-200 mg/kg and a slightly over-proportional increase in AUC under one dosage interval after the first dose and at steady-state. The PK profiles at all dose levels tested were adequately described by a linear PK model, indicating that the clearance process was not saturated at doses up to 200 mg/kg given twice weekly.

1.3.2.1 FS118 Single Dose PK

In this single i.v. dose study, the clearance of 4 mg/kg FS118 was compared to single iv administration of an anti-hPD-L1 mAb. Note, this anti-hPD-L1 mAb had the same human IgG1 framework and a different anti-PD-L1 Fab binding moiety (clone S1) compared with FS118. The anti-hPD-L1 mAb displayed typical IgG kinetics over the first 7 days post-dose, followed by a rapid loss of exposure, indicative of an ADA response; in contrast, FS118 exhibited a markedly faster clearance. Both FS118 and the anti-hPD-L1 mAb bind to PD-L1, although similarity of the binding epitope is unknown. This phenomenon has not been observed with other mAb² indicating that the mAb² format as such is not responsible. Instead, it appears that the targeted changes in the permissive residues in the CH3 domain of FS118 and/or LAG-3 binding is responsible for the higher clearance rate of FS118 as compared with the anti-hPD-L1 mAb.

1.3.2.2 FS118 Dose Range Finding (DRF) Study

In the DRF study (once weekly i.v dosing of 10, 50 and 200 mg/kg for 4 wks), the measurements of plasma FS118 and plasma ADA were performed as described in Examples 1.2.1 and 1.2.2 respectively. Exposure to FS118 (AUC) was decreased after the last dose; this was particularly apparent for the 10 mg/kg dose group and indicative of an immune response, which was confirmed by the presence of ADAs. Due to this immune response, in the DRF study, exposure to FS118 was not maintained throughout the dosing interval at the 10 and 50 mg/kg dose groups nor for ⅓ animals in the 200 mg/kg dose group. This phenomenon is not uncommon for a human IgG administered to NHP due to expected immunogenicity responses to human IgG in an NHP model.

1.3.2.3 FS118 4 wk GLP Toxicity Study

Since maintenance of exposure was unlikely to be achieved after repeated doses <50 mg/kg/wk, FS118 was dosed i.v. twice weekly at 60 and 200 mg/kg in the 4 wk GLP toxicity study. Whilst all treated animals in the 4 wk GLP toxicity study developed an ADA response, there was little impact on FS118 exposure and an adequate exposure margin was maintained compared with the predicted clinical exposure.

There was no significant accumulation of FS118 after repeated twice weekly dosing and no impact of gender on the PK of FS118.

1.3.2.4 Total Soluble PD-L1 (sPD-L1)

Plasma levels of total sPD-L1 were measured in the DRF and 4 wk GLP toxicity studies as described in Example 1.2.3. The capture of sPD-L1 is indicative of target engagement if the clearance of sPD-L1-FS118 complex is slower than the clearance of sPD-L1, leading to an increase in the level of sPD-L1-F5118 complex in the plasma. Although membrane bound PD-L1 is the primary target, the increase in total systemic sPD-L1 may be a potential biomarker of target saturation.

A ≥10 fold increase in plasma total sPD-L1 was observed within 24 h after administration of FS118 at doses ranging from 10 to 200 mg/kg. In the DRF study there was a similar trajectory of increase in total sPD-L1 over 0-96 h after the first dose in all three dose groups; with a continuous increase in total sPD-L1 over the first dosing interval only for the 200 mg/kg dose group. In this study, any further analysis of the increase in total sPD-L1 beyond 7 days after the first dose was compromised by the presence of ADAs to FS118. At the higher exposure levels achieved in the 4 wk GLP toxicity study, mean total sPD-L1 capture continued to increase over the duration of the study, with a large inter-animal variability, especially for the 200 mg/kg dose group. A plateau (indicative of maximum target capture) was not observed until 2-4 wks after treatment with either 60 or 200 mg/kg twice weekly.

Given the high variability it is not possible to conclude that maximum target capture was achieved in this study. However, as expected, loss of FS118 exposure in the recovery animals was clearly associated with loss of sPD-L1 capture when the FS118 concentration fell below 10 μg/mL. Apart from a transient drop in FS118 exposure at study day 22 for some animals in the 60 mg/kg dose group, plasma FS118 was maintained above 10 μg/mL throughout the dosing period for both the 60 and 200 mg/kg dose groups, implying that PD-L1 suppression was also maintained during this period.

It should be noted that, when expressed on a molar basis, plasma sPD-L1-FS118 complex (i.e. total sPD-L1) is only a small fraction of the total FS118 concentration. For example, mean FS118 trough concentration after repeated administration of 200 mg/kg twice weekly is 220 μg/mL (1.5 μM); mean total sPD-L1 concentration at the same time point is −5 ng/mL (0.2 nM). Consequently, systemic FS118-PD-L1 complex cannot be responsible for the clearance rate of FS118.

In conclusion, a rapid increase in total sPD-L1 was observed with all dose levels of FS118 with the rate of increase being similar across all dose levels. No definitive conclusion could be drawn concerning whether maximum target capture had been achieved. It is likely, however, that target engagement was saturated at 10 mg/kg FS118 and above. Total sPD-L1 values returned to baseline by the end of the recovery period. A threshold of FS118 above 10 μg/mL in the plasma of the animal was associated with sustained increased total sPD-L1 levels.

1.4 Investigation of Clearance of FS118 and mLAG-3/PD-L1

Overall, the available PK data suggested that the rate of clearance of mLAG-3/PD-L1 mAb² in wt and LAG-3 KO mice was primarily a consequence of the combination of functional PD-L1 and the target-specific changes of the permissive residues in the CH3 domain to enable binding to LAG-3. The observed clearance rate of FS118 in NHP was most likely driven by the same mechanism, although a contribution from the higher affinity LAG-3 binding in NHP and human (versus mice) cannot be excluded.

Additional factors which may contribute to this rate of clearance were investigated and are summarised below:
FS118 displayed the expected pH-sensitive binding characteristics to FcRn and this was not influenced by binding to LAG-3.
Tissue cross-reactivity studies with FS118 in NHP and human tissues did not show any off-target binding which could explain the observed clearance of FS118. In addition, FS118 did not show any binding to closely related targets.
FS118 maintained functional activity in serum when incubated at 37 degC for up to 15 days, indicating that catabolism is unlikely to be an explanation.
Incubation of FS118 and the mouse surrogate mAb² with activated human and mouse T cells, respectively, did not result in internalisation of test article over a 3-hour incubation period compared with an anti-CD3 antibody. These results indicate that clearance of FS118 was not mediated by internalisation, although target engagement and internalisation by target expressed on other cells has not been assessed.

Overall, these data indicate that the non-saturable rate of clearance of FS118 and the mouse surrogate mAb² is PD-L1 target driven and associated with the LAG-3-targeted CH3 modifications in the mAb² construct, although the potential for an additional contribution from LAG-3 binding cannot be excluded in NHP. Due to FS118 having similar binding properties for NHP PD-L1 & LAG-3 and human PD-L1 & LAG-3, a similar clearance rate was predicted in human.

1.5 Predicted Pharmacokinetic-Pharmacodynamic Behaviour in Human

1.5.1 NHP PK Model

A 2-compartment population PK model describing the systemic exposure to FS118 was constructed from the NHP single-dose PK, DRF and GLP studies PK data (0-7 days post-dose; see Examples 1.3.2.1-1.3.2.3). All PK modelling, fitting, and simulations were performed using ADAPT version 5 (D'Argenio et al 2009).

Each individual animal's PK was initially fit to a two-compartment model, which resulted in four PK parameters (CL1, CL2, V1, and V2) per animal. This was done to assess whether all animals' PK could be pooled together and used as part of a population PK model.

The population PK fit was performed under the assumption that each animal's PK parameters were drawn from a log-normal distribution, characterized by a certain population mean vector and covariance matrix. This reduced the number of unknowns dramatically, from 4×(number of animals)=112 in the individual PK case to 14 (four population mean values and 10 distinct covariance matrix elements for a 4×4 covariance matrix), thereby significantly improving the known-to-unknown ratio.

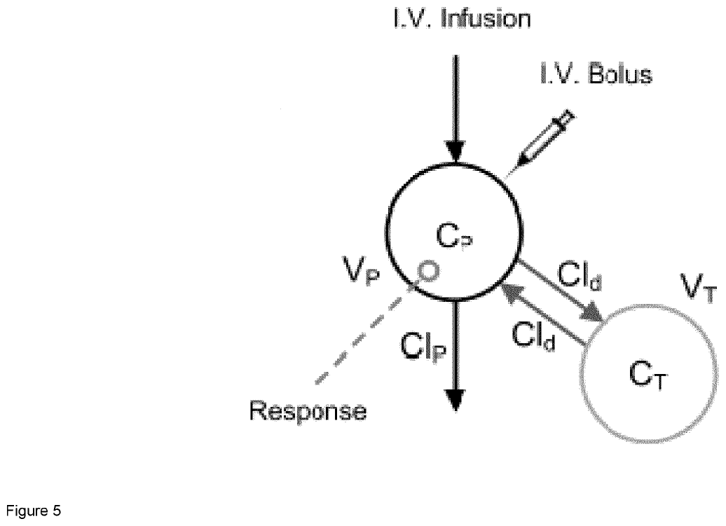

The overall structure of the NHP and human PK model describing the linear kinetics of FS118 is shown in FIG. 5. The model well described the observed data in NHP (Table 4) and predicted the observed data after repeated administration in the 4 wk GLP toxicity study; in other words, there was no evidence to suggest a significant saturable component in the clearance of FS118. This model was allometrically scaled to predict the human PK of FS118, using exponents of 0.75 for clearance and inter-compartmental exchange and 1.0 for volume (Table 4). Since target-mediated kinetics were not observed, target binding affinity was not incorporated into the PK model. Given these assumptions, FS118 exposure in human was predicted for different dosing regimens. Using these parameter values, a simulation of PK in 1000 human subjects was performed to assess the PK range in a human population prior to the First-in-human (FIH) clinical trial. The simulations further predicted that doses of 20 mg/kg and below would generate FS118 exposure levels in vivo which would be well below the Highest Non-Severely Toxic Dose (HNSTD; see Example 1.5.2 below) and thus these doses presented no safety concern.

It should be noted that the observed rate of clearance for FS118 in NHP is higher than typically observed for a monospecific antibody in NHP but not so high as to preclude a pharmacological effect.

TABLE 4
FS118-PK model parameters

| PK parameter | Unit | NHP | Assumption: scaling to human | Human |
|---|---|---|---|---|
| Central Volume (V1) | mt | 132 | exponent = 1; typical for protein therapeutic | 3209 |
| Peripheral volume (V2) | mt | 96 | exponent = 1; typical for protein therapeutic | 2326 |
| Exchange coefficient (CL2) | mL/h | 1.65 | exponent = 0.75; typical for protein therapeutic | 18.06 |
| FS118 clearance (CL1) | mL/h | 7.22 | exponent = 0.75; typical for protein therapeutic | 79.04 |

Body weight NHP 2.88 Kg; Human 70 Kg
Source data: [F-Star Study Report 022, Tables 7 and 8]

1.5.2 Highest Non-Severely Toxic Dose (HNSTD)

FS118 was well tolerated in the NHP 4 wk GLP toxicity study (see Example 1.3.2.3) and the HNSTD was established to be 200 mg/kg twice weekly. No FS118-related increase in cytokines was observed in in vitro assays, using either the wet-coated immobilized format with human PBMCs or the human whole blood format. In addition, there was no observed FS118 treatment-related increase in a panel of serum cytokines (IL-2, IL-6, IL-8, IL-10, IFN-γ and TNF-α) in the NHP 4 wk GLP toxicity study.

ICH S9 guidance (ICH S9) recommends initial clinical dosing at ⅙^{th} the HNSTD (Table 5) for FIH studies in advanced cancer patients; however, a recent publication from the FDA suggests that this may not be appropriate for immuno-oncology drugs and additional factors relating to target occupancy and functional activity should be considered (Saber et al 2016).

The proposed FIH starting flat dose of 800 μg rising to 20 mg/kg weekly dosing (see Example 1.5.3.1) is at least 10 times below the HNSTD, well below the recommended ICH S9 guidance.

TABLE 5
FDA Guidance: S9 nonclinical evaluation anticancer pharmaceuticals

| Step* | Description | Dose (mg/kg) |
|---|---|---|
| 1 | HNSTD | 200 |
| 2 | l/6th HNSTD for non-rodent | 33.3 |

*ICH S9 (FDA Guidance for Industry Mar 2010)

1.5.3 FIH Study Design

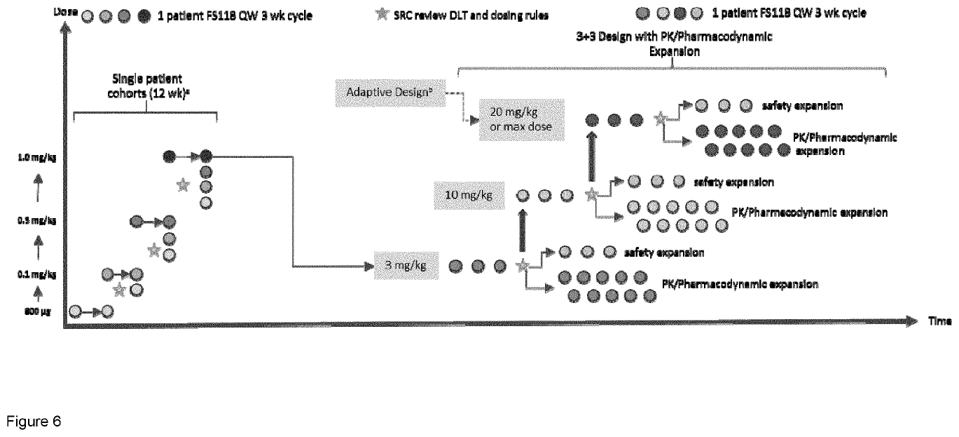

The First in Human study (Example 2) was designed as an open-label, multiple dose, dose-escalation and cohort expansion study. It was decided to conduct the study in adult patients diagnosed with advanced tumours to characterize the safety, tolerability, pharmacokinetics (PK), and activity of FS118. It was further decided that initial patients would be recruited into an accelerated titration design, where single patient cohorts would be evaluated, followed by a 3+3 ascending dose escalation design (FIG. 6). The study was designed to systematically assess safety and tolerability, and to identify the maximum tolerated dose (MTD) and/or recommended Phase 2 dose (RP2D) for FS118 in patients with advanced tumours. The RP2D was defined as the maximum biological effective dose with acceptable toxicity.

Dose increments between the starting dose and the highest dose were selected to allow for safe dose escalation and were guided by PK modelling to capture an adequate FS118 dose-exposure relationship. It was decided to assess PK in humans using a validated Gyros assay which measures free FS118 (LAG-3 capture/PD-L1 detection format) and to ensure that PK data would be available at the end of the intra-patient dose-escalation phase to allow assessment of the dose-exposure relationship compared with the predicted human PK. It was also decided to measure the increase in total plasma sLAG-3 and sPD-L1, as potential biomarkers of target engagement and to assess the potential for generation of ADAs from samples taken after each 3 wk treatment cycle.

1.5.3.1 Justification of FIH Starting Dose and Dose Escalation Strategy

In setting an acceptable starting dose for clinical testing it was important to consider the potential pharmacological activity as well as publically available clinical experience with similar molecules (Saber et al 2016). Based on all the available data, the proposed FIH starting dose was 800 μg intravenously and a within-patient accelerated dose escalation phase was proposed in order to safely increase FS118 exposure to that anticipated for anti-tumour efficacy; minimizing exposure to potentially ineffective dosing regimens. The FIH study was also designed to investigate the need for dosing regimens which maintain target suppression throughout the dosing interval.

The key points supporting the selected dosing strategy were as follows:
Exposure data from a mouse syngeneic tumour model with the mouse surrogate mAb², suggest that continuous high exposure to FS118 is not required for anti-tumour efficacy and this is in sharp contrast to a monospecific anti-PD-L1 mAb. In this tumour model, doses of the mouse surrogate mAb² mg/kg were associated with inhibition of tumour progression, with doses of 3, 10 and 20 mg/kg being statistically significant. Anti-tumour efficacy for an anti-PD-L1 mAb and the mouse surrogate mAb² administered at 10 mg/kg every 3 days (3 doses) and exposure profiles after a single dose of both molecules in wt non-tumour bearing mice are shown in FIG. 3A. Whilst exposure to the anti-PD-L1 mAb was maintained above 100 μg/mL over a 3-day period, exposure to mLAG-3/PD-L1 fell to about 10 μg/mL in the same time-period. It should be noted that in the MC38 model trough exposure to the mouse surrogate mAb² decreased over time, possibly related to ADA formation. At 3 mg/kg the mouse surrogate mAb² every 3 days, estimated Cₘₐₓ after the first dose was 25 μg/mL and the observed Cₘₐₓ after the last dose was 6 μg/mL.
FS118 was well tolerated in the NHP 4 wk GLP toxicity study. Mixed mononuclear cell infiltration in the brain and other tissues was observed, similar to that observed with other immune checkpoint inhibitors.
No FS118-related increase in cytokines was observed in in vitro assays and there was no observed increase in serum cytokine levels in the NHP 4 wk GLP toxicity study. This is consistent with other immuno-oncology biotherapeutics where doses associated with >90% target occupancy are not associated with acute cytokine release syndrome (Herbst et al 2014, Heery et al 2017).
The Biacore binding affinity of the mouse surrogate mAb² to mPD-L1 has been shown to be about 10-fold higher when compared with the binding of FS118 to hPD-L1. In contrast, the Biacore binding affinity of the mouse surrogate mAb² to mLAG-3 has been shown to be about 20-fold lower when compared with the binding of FS118 to hLAG-3. However, these differences were much less apparent when comparing EC₅₀ values for binding to HEK cells overexpressing the respective target proteins and EC₅₀ values in a functional T cell activation assay. Given the similar rate of clearance for FS118 and the mouse surrogate mAb², in the presence of functional PD-L1 target binding, these observed differences in target binding affinity are unlikely to affect the prediction of FS118 PK in human.
Analysis of available non-clinical and clinical safety data for immuno-oncology drugs shows that FIH doses based on either 20-80% target occupancy and/or 20-80% in vitro functional activity have acceptable clinical toxicity. FIH systemic exposure above target saturation were also acceptable for antibodies with either normal or silenced ADCC activity (Saber et al 2016) and it should be noted that FS118 has the LALA mutation to reduce ADCC activity. Estimates of systemic target occupancy and in vitro functional activity (as a percentage of maximum) were between 35.8% and 79.2% for Cₘ, (0.26 μg/mL) at the proposed starting dose of 800 μg and were considered to be appropriate for the FIH dose.
In a human T cell activation assay with sub-optimal activation, FS118 stimulated IFNγ production with a mean EC₅₀ of 0.22 μg/mL, although considerable variability was observed.
FS118 was shown to have a high clearance rate compared with monospecific antibodies to the same targets and the mechanism appeared to be mainly driven by the PD-L1 binding component in this bispecific construct, at least in the mouse. Note that FS118 had normal IgG kinetics in the wt and LAG-3 KO mouse (no functional PD-L1 binding), whereas the surrogate mAb² was cleared quickly in both the wt and LAG-3 KO mouse. The clearance process was not saturated at doses up to 200 mg/kg in NHP and the projected terminal half-life in human was 3.7 days (95% confidence interval 0.35-10.4 days).
Cₘₐₓ and AUC (under one 7-day dosage interval) at steady-state for the HNSTD in NHP 4 wk toxicity study were compared with the predicted exposure at steady-state in human for each dose in the proposed dose escalation regimen and the resulting exposure safety margins are shown in Table 6. The proposed 800 μg starting dose (˜11 μg/kg for a 70-kg subject) is anticipated to give a >15,000-fold lower exposure than the HNSTD in NHP and this reduces to >190-fold at the end of the within-patient accelerated titration phase. The large safety margin for the FIH dose allows for FS118 to have unexpected normal IgG kinetics in human and the PK behaviour of FS118 in human will be confirmed prior to proceeding to the dose escalation part of the study. In the clinic, a dose of 20 mg/kg/wk is anticipated to give a C_{trough} concentration of FS118>10 μg/mL and a 10-fold lower exposure (Cₘₐₓ and AUC) than the HNSTD in NHP. Doses and dosing frequency to achieve this target concentration may be adjusted at the end of the accelerated dose titration phase.

TABLE 6
Predicted exposure margins: FIH study
Predicted exposure margins-human vs NHP

| | | | Exposure margin | |
|---|---|---|---|---|
| Dose | Frequency | Route | Cmax | AUC |
| 800 μg | q1wk | iv | 16,735 | 17,095 |
| 0.1 mg/kg | q1wk | iv | 1,911 | 1,948 |
| 0.3 mg/kg | q1wk | iv | 636 | 645 |
| 1.0 mg/kg | q1wk | iv | 191 | 194 |
| 3 mg/kg | q1wk | iv | 63 | 65 |
| 10 mg/kg | q1wk | iv | 19 | 19 |
| 20 mg/kg | q1wk | iv | 9 | 10 |

Predicted exposure margin, Cmax and AUC (steady-state)
compared with Cmax and AUC (0-7 days, steady state) at the highest dose tested in the 4 wk GLP tox study: 200 mg/kg/twice weekly NHP (i.e. observed AUC(0-tau) × 2)

Plasma total sPD-L1 was not included in the PK model although there is evidence from NHP to suggest that this may be a good biomarker of target engagement and this will be measured in the FIH study. The mouse syngeneic tumour model also suggests that total suppression of PD-L1 may not be required throughout each treatment cycle. In the NHP, plasma FS118 concentration 10 μg/mL was associated with maintenance of PD-L1 capture (and by inference, PD-L1 suppression) and the FIH clinical study was designed to explore both maximum suppression of PD-L1 for a limited time-period (Cₘₐₓ≥10 μg/mL) and continuous suppression of PD-L1 throughout each dosing cycle (C_{trough}≥10 μg/mL).

Overall, these data indicated that 800 μg was an appropriate starting dose to initiate clinical testing of FS118. This proposed starting dose was projected to give a maximum concentration (Cₘₐₓ 0.26 μg/mL) at the end of the 1-hour infusion, which is acceptable in terms of target receptor occupancy and in vitro functional activity. Furthermore, this Cₘₐₓ is about 10- to 100-fold lower than the Cₘₐₓ associated with anti-tumour efficacy for the FS118 mAb² surrogate molecule and >15,000 fold lower than the Cₘₐₓ exposure to FS118 at the HNSTD in NHP; similar exposure margins are maintained for AUC under a dosing interval (Table 6). A within-patient dose escalation scheme is proposed to achieve therapeutically relevant exposure to FS118 rapidly and safely, minimising exposure of patients to sub-therapeutic doses while maintaining safety. At the end of the within-patient dose escalation phase, mean Cₘₐₓ was anticipated to be 25 μg/mL, which is within the exposure range associated with anti-tumour efficacy for the mouse surrogate mAb² in the MC38 tumour model and above the FS118 exposure (10 μg/mL) associated with maintenance of sPD-L1 capture in NHP.

Although the non-clinical tumour efficacy data suggested that continuous suppression of PD-L1 is not required, it was decided to also explore doses of FS118 which maintain PD-L1 capture throughout the dosing interval during the FIH study and doses of FS118 mg/kg/wk were anticipated to give a mean C_{trough} concentration 10 μg/mL.

1.6 Summary and Conclusions

Exposure data from a mouse syngeneic tumour model with the mouse surrogate mAb² (mLAG-3/PD-L1), suggested that continuous high exposure to FS118 was not required for anti-tumour efficacy, in contrast to a monospecific anti-PD-L1 mAb. In this tumour model, doses of the mouse surrogate mAb² mg/kg were associated with inhibition of tumour progression, with doses of 3, 10 and 20 mg/kg being statistically significant.

In the presence of functional PD-L1 binding, the rate of clearance of both FS118 and the mouse surrogate mAb² is higher than observed for a standard monospecific IgG-like molecule. Although there is a slight over-proportional increase in exposure with increasing dose, this clearance process does not appear to be saturated at doses up to 200 mg/kg twice weekly in NHP and is adequately described by a linear PK model. In other words, FS118 does not display saturable target-mediated kinetic behaviour, as sometimes observed with IgG-like molecules targeted to a membrane receptor. However, this clearance process appears to be dependent on functional PD-L1 binding of the mAb² construct, since normal IgG kinetics are observed for FS118 in wt and LAG-3 KO mice, (FS118 lacks significant PD-L1 binding in the mouse).

In the NHP 4 wk GLP toxicity study, FS118 has been shown to be well tolerated at doses which provide adequate exposure margins for clinical testing. The proposed FIH starting dose of 800 μg is projected to give a maximum concentration (Cₘₐₓ) at the end of the 1 hour infusion of 0.26 μg/mL, which is about 10-fold lower than the Cₘₐₓ associated with anti-tumour efficacy for the mouse surrogate mAb² molecule and >15,000 fold lower than Cₘₐₓ exposure to FS118 at the HNSTD in NHP. Similar exposure margins are maintained for AUC under a dosing interval.

Plasma total sPD-L1 has been shown to be a useful biomarker of PD-L1 target engagement in NHP. In the NHP, plasma FS118 concentration 10 μg/mL is associated with maintenance of PD-L1 capture (and by inference, PD-L1 suppression) and the FIH clinical study was designed to explore both maximum suppression of PD-L1 for a limited time period (FS118 Cₘₐₓ≥10 μg/mL) and continuous suppression of PD-L1 throughout each dosing cycle (FS118 C_{trough}≥10 μg/mL). The dose escalation strategy was designed to achieve a Cₘₐₓ of about 10 μg/mL at the end of the within-patient accelerated dose titration phase (at 1 mg/kg/wk FS118) and then to explore higher exposure levels which maintain FS118 10 μg/mL within the dosing interval. Doses of 10 and 20 mg/kg/wk were predicted to achieve mean plasma FS118 concentrations >10 μg/mL throughout the dosage interval.

### Example 2: Phase I, Open-Label, Dose-Escalation, and Cohort Expansion First-In-Human Study of the Safety, Tolerability, Pharmacokinetics, and Activity of FS118, a LAG-3/PD-L1 Bispecific Antibody, in Patients with Advanced Malignancies that have Progressed on or after Prior PD-1/PD-L1 Containing Therapy

2.1 Study Design & Parameters

This study was conducted in adult patients diagnosed with advanced tumours to characterize the safety, tolerability, pharmacokinetics (PK), and activity of FS118. This Phase I, multi-center, open-label, multiple-dose, first-in-human study initiated with an accelerated titration design (during which single-patient cohorts were evaluated) followed by a 3+3 ascending dose-escalation design. The study was designed to systematically assess safety and tolerability, and to identify the maximum tolerated dose (MTD) and/or recommended Phase 2 dose (RP2D) for FS118 in patients with advanced tumours. The RP2D was defined as the maximum biological effective dose with acceptable toxicity. Pharmacokinetics, pharmacodynamics, immunogenicity, and response were also assessed.

Following informed consent, all patients underwent screening to determine eligibility within 28 days prior to the start of treatment. Dosing of patients occurred intravenously (IV) weekly in 3-week treatment cycles until iCPD (i.e., immune-confirmed progressive disease) (or progressive disease per the Lugano classification for patients with lymphoma), unacceptable toxicity, withdrawal of consent by patient, discontinuation of patient by Investigator, Sponsor decision to terminate the study or treatment, initiation of alternate anti-cancer therapy, or death. Patients received or will have an End-of-Treatment (EOT) visit approximately 28 days (±7 days) after the last dose of FS118 and a 90-day Follow-up visit approximately 90 days (±7 days) after the last dose of FS118. For all patients after documented iCPD (or progressive disease per the Lugano classification for patients with lymphoma), overall survival (OS) was or will be assessed every 3 months to assess survival and post-study cancer therapy administered.

The first 5 cohorts enrolled sequentially as single-patient cohorts, and patients were observed for dose-limiting toxicities (DLTs) during Cycle 1. As no DLT or ≥Grade 2 adverse event that was not clearly attributed to the patient's underlying disease, other medical conditions, or concomitant medications or procedures were observed in each cohort, a new patient was dosed in the next higher dose cohort and observed for the DLT period. After completion of Cycle 1 in cohort 5 without a DLT or Grade 2 adverse event that was not clearly attributed to the patient's underlying disease, other medical conditions, or concomitant medications or procedures, the dose-escalation regimen continued as a 3+3 design from cohort 6 onward. Intra-patient dose escalation proceeded in single-patient cohorts if the patient tolerated their initial dosing, the patient(s) in the next higher dose cohort had completed the DLT period without evidence of a DLT or a ≥Grade 2 adverse event that was not clearly attributed to the patient's underlying disease, other medical conditions, or concomitant medications or procedures, and the dose has been declared safe by the Safety Review Committee (SRC).

If in any of the single-patient cohorts a patient experiences a Grade 2 adverse event that is not clearly attributed to the patient's underlying disease, other medical conditions, or concomitant medications or procedures during the DLT period, an additional 2 patients would have been enrolled at that dose level and evaluated using 3+3 design rules but this did not occur. All subsequent cohorts enrolled in a 3+3 design. If a DLT occurred, the cohort would have been expanded to 6 patients but no DLTs were observed.

Toxicity was evaluated according to National Cancer Institute (NCI) Common Terminology Criteria for Adverse Events (CTCAE), Version 4.03.

Primary Objectives

The primary objectives of this study were:
1. To assess the safety and determine the MTD and/or RP2D of FS118, and
2. To determine the PK parameters of FS118.

Secondary Objectives

The secondary objectives of this study were:
1. To assess preliminary evidence of anti-cancer activity of FS118 per Response Evaluation Criteria in Solid Tumours (RECIST) 1.1 or the Lugano classification, as applicable, and iRECIST (modified RECIST 1.1 for immune-based therapeutics); and
2. To characterize the immunogenicity (anti-drug antibodies [ADAs]) of FS118.

Exploratory Objective

The exploratory objective of this study was to characterize the pharmacodynamic profile and correlate potential primary pharmacology with exposure.

Patient Population and Number of Patients

24 patients were enrolled by May 2019, increasing to 40 patients by August 2019 and to 43 patients by April 2020. Patients were enrolled across 4 study sites in the United States. Patients were aged ≥18 years with advanced tumours.

Treatment Administration

FS118 was administered intravenously to the first cohort as a slow bolus injection and by continuous infusion pump to subsequent cohorts weekly in 3-week treatment cycles until iCPD (or progressive disease per the Lugano classification for patients with lymphoma), unacceptable toxicity, withdrawal of consent by patient, discontinuation of patient by Investigator, Sponsor decision to terminate the study or treatment, initiation of alternate anti-cancer therapy, or death.

Duration of Treatment and Study

Patients were considered to have completed treatment if they completed 16 cycles (or 12 months) of FS118 treatment, or if they experience confirmed progressive disease. The final analyses for the primary endpoint will be conducted, and a single final clinical study report compiled, after all patients enrolled in the study have had the opportunity to complete 16 cycles of treatment with FS118 and be followed for 90 days after the last study drug administration. The estimated time frame for study completion is 36 months.

Eligibility Criteria

Each patient enrolled had to meet all of the following further requirements to be eligible to enroll in the study:
1. For dose escalation: Patients with histologically confirmed, locally advanced, unresectable, or metastatic solid tumours or haematological malignancies that progressed while on or after anti-programmed cell death protein 1 (PD-1) or anti-programmed death-ligand 1 (PD-L1) therapy for whom no effective standard therapy is available or standard therapy has failed;
2. For dose expansion: Patients with histologically confirmed, locally advanced, unresectable, or metastatic cervical, ovarian, bladder, renal, head and neck squamous cell carcinoma, melanoma, non-small cell lung cancer, triple-negative breast cancer, or non-Hodgkin or Hodgkin lymphoma that progressed while on or after anti-PD-1 or PD-L1 therapy for whom no effective standard therapy is available or standard therapy has failed;
3. Minimum treatment duration of prior PD-1 or PD-L1-containing regimen was 12 weeks (or equivalent of 2 response evaluations);
4. Measurable disease (defined as at least 1 measurable lesion outside the central nervous system [CNS]), as determined by the Investigator using RECIST 1.1 or the Lugano classification, as applicable;
5. Eastern Cooperative Oncology Group (ECOG) Performance Status 1;
6. Life expectancy estimated to be at least 3 months;
7. The patient agreed to undergo a pre-treatment and on-treatment biopsy of the tumour and the biopsy procedure was not judged to be high-risk by the Investigator. For patients in the single-patient cohorts, acceptable baseline tumour samples included newly obtained tumour biopsy samples and/or archival tissue samples (<6 months old) from original diagnosis, if available;
8. Highly effective contraception (that is, methods with a failure rate of less than 1% per year) for both male and female patients if the risk of conception existed. Highly effective contraception had to be used 28 days prior to first study treatment administration, for the duration of study treatment, and at least for 60 days after stopping study treatment. Should a female patient have become pregnant or suspected she was pregnant while she or her partner was participating in this study, the treating physician and Sponsor (or designee) would be informed immediately; and
9. Willing and able to provide written informed consent.

Patients who fulfilled any of the following criteria at Screening were not eligible for admission into the study:
1. Received systemic anti-cancer chemotherapy within 28 days or 5 half-lives, whichever is shorter, of the first dose of study drug, prior treatment with more than 1 immune checkpoint inhibitor (except as a combination in approved indications) that was not standard of care, or prior treatment with a lymphocyte-activation gene 3 (LAG-3) inhibitor or multi-specific immune checkpoint inhibitor molecules;
2. Patients with active autoimmune disease requiring treatment in the previous 2 years and patients with a documented history of any autoimmune disease. Note: This included patients with a history of inflammatory bowel disease, ulcerative colitis and Crohn's Disease, rheumatoid arthritis, systemic progressive sclerosis (scleroderma), systemic lupus erythematosus, autoimmune vasculitis (e.g., Wegener's granulomatosis), CNS or motor neuropathy considered of autoimmune origin (e.g., Guillain Barré Syndrome, Myasthenia gravis, multiple sclerosis), and moderate or severe psoriasis. However, patients with rheumatoid arthritis or psoriasis in stable remission for at least 6 months and without contraindications to possible co-treatment with corticosteroids for immune-related adverse events, vitiligo, Sjogren's Syndrome, interstitial cystitis, Graves' or Hashimoto's Disease, or hypothyroidism stable on hormone replacement were allowed with the study Medical Monitor's approval;
3. History of uncontrolled intercurrent illness including but not limited to:
   Documented hypertension uncontrolled by treatment with standard therapies (not stabilized to 150/90 mmHg or lower), or
   Documented uncontrolled diabetes. Note: Patients with well-controlled diabetes under stable insulin replacement therapy for at least 6 months and without contraindications to possible co-treatment with corticosteroids for immune-related adverse events could be considered;
4. Known infections:
   Human immunodeficiency virus, hepatitis B virus (HBV) (i.e., hepatitis B surface antigen-positive), or hepatitis C virus (HCV) (i.e., detectable HCV ribonucleic acid [RNA]). Note: Patients with a prior history of treated HBV infection who are antigen negative or patients with a prior history of treated HCV infection who are HCV RNA-undetectable could be considered; or
   Active infections (including asymptomatic infections with positive virus titers and Investigator's judgment that worsening of condition is likely with study treatment or condition would impair/prohibit a patient's participation in the study;
5. Uncontrolled CNS metastases, primary CNS tumours, or solid tumours with CNS metastases as only measurable disease. Patients with active disease but stable CNS disease could be enrolled;
6. Prior history of or active interstitial lung disease or pneumonitis, encephalitis, seizures, severe immune-related adverse events (i.e., pneumonitis, hepatitis, colitis, hypophysitis, pancreatitis, myocarditis, CNS, or ophthalmic) with prior PD-1/PD-L1 containing treatments, history of severe or life-threatening skin adverse reaction on prior treatment with other immune stimulatory anticancer agents;
7. Use of immunosuppressive agents, prior organ transplantation requiring immunosuppression, hypersensitivity or intolerance to monoclonal antibodies or their excipients, or persisting toxicity related to prior therapy of >Grade 1 NCI CTCAE v4.03 with the following exceptions:
   All grades of alopecia were acceptable;
   Endocrine dysfunction on replacement therapy was acceptable (including stable hypophysitis on hormone replacement therapy);
   Non-systemic steroids; topical, intraocular, intranasal, intraarticular, or inhalative steroids were allowed;
   Systemic steroid replacement treatment at or below 10 mg/day prednisone equivalent in patients with adrenal insufficiency was allowed; and
   Enrolment of patients that receive systemic steroid treatment at or below 10 mg/day prednisone equivalent as part of their palliative treatment or symptomatic disease control was to be discussed with the Medical Monitor and/or Sponsor;
8. Significant cardiac abnormalities, including a history of long QTc syndrome and/or pacemaker, cerebral vascular accident/stroke (<6 months prior to enrolment), myocardial infarction (<6 months prior to enrolment), unstable angina, congestive heart failure (New York Heart Association Classification Class ≥II), or clinically significant and symptomatic cardiac arrhythmia that had not been controlled with medication for at least 6 months;
9. Screening laboratory values with the following criteria (using NCI CTCAE, Version 4.03):
   Hemoglobin <9.0 g/dL (5.7 μmol/L);
   Absolute neutrophil count (ANC)<1.0×10⁹/L;
   Platelets <100×10⁹/L;
   Serum creatinine >1.5× upper limit of normal (ULN);
   Total bilirubin >1.5×ULN; or aspartate aminotransferase (AST) and alanine aminotransferase (ALT) >2.5×ULN (5×ULN if liver metastasis); or
10. Intolerance to the investigational product or its excipients, or any condition that would significantly impair and/or prohibit the patient's participation in the study, as per the Investigator's judgment.

Main Criteria for Evaluation and Analyses

The primary endpoints of this study were:
Incidence, severity, and duration of adverse events; and
PK parameters, including maximum observed concentration (Cₘₐₓ), time to Cₘₐₓ (Tₘₐₓ), observed trough serum concentration (C_{trough}), 1 terminal elimination half-life (t_{1/2}), area under the concentration-time curve (AUC) in 1 dosing interval [AUC(TAU)], average concentration over a dosing interval [AUC(TAU)/tau], systemic clearance (CL), volume of distribution at steady-state (Vₛₛ), and accumulation ratio from first dose to steady-state.

The secondary endpoints of this study were:
Response as assessed by RECIST 1.1 or the Lugano classification, as applicable, and iRECIST. These responses have been used to determine the disease control rate (DCR), objective response rate (ORR), duration of response (DoR), and progression-free survival (PFS)/iPFS. Overall survival will also be assessed; and
Incidence of FS118 immunogenicity, including ADA detection and analysis.

The exploratory endpoints of this study included:
Percentage PD-L1 and LAG-3 receptor occupancy in CD3+, CD4+, and CD8+ T cell populations by flow cytometry of peripheral blood mononuclear cells;
Soluble PD-L1 and LAG-3 quantification.

Statistical Considerations

Patient disposition has been tabulated for all enrolled patients. Demographic and baseline data (i.e., age, gender, race, ethnicity, height, and weight), and disease history and characteristics were summarized using descriptive statistics for the Safety Analysis Set.

Efficacy analyses have been conducted using the Efficacy Analysis Set. Tumour response data per RECIST 1.1 criteria or the Lugano classification, as applicable, and per iRECIST criteria have been employed. For ORR and DCR, the point estimates and the 95% exact confidence intervals have been/will be provided. Patients with unknown or missing response will be treated as non-responders (i.e., they will be included in the denominator when calculating the percentage).

Best overall response has been determined according to RECIST 1.1 criteria or the Lugano classification, as applicable, and according to iRECIST criteria.

Time-to-event variables, including duration of cure, DoR, PFS, iPFS, and OS, have been/will be summarized descriptively using the Kaplan-Meier method. Censoring methods for time-to-event variables will be described in the Statistical Analysis Plan. Kaplan-Meier curves for time-to-event variables will be generated.

The PK Analysis Set has been used for summaries of all PK data. Serum concentration versus time profiles have been presented, where necessary, graphically along with tabular summaries of non-parametric parameters Cₘₐₓ, Tₘₐₓ, C_{trough}, and AUC over the dosage interval for each patient and by dose cohort. If appropriate, total AUC has been calculated, using extrapolation to infinity from the terminal phase of the concentration versus time profile and allowing serum t_{1/2}, CL, and Vₛₛ to also be derived.

The Pharmacodynamic Analysis Set has been used for pharmacodynamic analyses.

The proportion of patients with positive FS118 ADA and the proportion of patients with positive neutralizing FS118 ADA during the study has been summarized. Correlation analysis of FS118 ADA titre and PK has been performed.

The safety profile has been based on adverse events (including DLTs and serious adverse events), physical examination findings (including ECOG performance status), vital sign measurements, standard clinical laboratory measurements, and electrocardiogram recordings.

Sample Size Justification

24 patients were enrolled by May 2019, increasing to 40 patients by August 2019 and to 43 patients by April 2020. The accelerated titration portion consisted of a minimum of 5 patients, and the 3+3 ascending dose-escalation portion of the study consisted of 3 to 6 patients per dose level. Cohorts could be expanded for safety reasons (up to 3 patients), to enrich for PK and/or pharmacodynamics (up to 10 patients), and to further characterize clinical efficacy at or before the RP2D level (up to 24 patients). The sample size for the study has been determined by practical considerations. No formal statistical assessment has been performed.

2.2 Interim Results (May 2019)

2.2.1 Interim Clinical Data

By May 2019, the single patient cohorts of the accelerated titration design (800 μg, 2400 μg, 0.1 mg/kg, 0.3 mg/kg, and 1.0 mg/kg doses) had been completed. In the 3+3 ascending dose-escalation design part of the Phase I study, the 3 mg/kg had been completed and dosing at the 10 mg/kg and 20 mg/kg dosage levels was ongoing. Two of the cohorts had been expanded (1 mg/kg to 3 subjects; 3 mg/kg to 10 subjects).

Of the 24 patients enrolled in the Phase I study by May 2019, 8 patients were active on treatment. 16 patients discontinued treatment, 4 patients due to iCPD, 2 patients due to progressive disease RECIST 1.1, and 10 patients due to other considerations: TBC/clinical PD/PI decision.

Of the treatment emergent adverse events observed during the Phase I study up to May 2019, 20% were assessed as related to FS118, all of which were mild to moderate. Of the 10 serious adverse events observed, none were related to FS118. No DLTs were observed at any of the dosages tested. This demonstrated that doses of up to 20 mg/kg are well tolerated and that the safety profile of FS118 is in-line with other immune checkpoint blockers.

Duration of treatment with FS118 continued for an average 9.2 weeks=3 cycles (0-24 weeks). For 14 subjects, at least 1 “on-study” scan was reported. Of the 14 patients, 5 had stable disease and 9 had progressive disease. Although determination of efficacy was not a primary objective of the Phase I study, the average time patients spent on FS118 treatment, and the fact that on-study scans showed disease stabilization in 5 out of 14 patients, demonstrated that FS118 is capable of disease stabilization and thus has the potential to inhibit tumour growth in human patients. In evaluating this data, it should be borne in mind that the patients enrolled in the Phase I study all had advanced malignancies and may have been too compromised to be capable of benefitting from treatment with FS118. Treatment of less compromised cancer patients, as may be investigated in a Phase 2 study, may show even higher efficacy of FS118.

2.2.2 Interim Pharmacokinetic/Pharmacodynamic Data

A Pharmacokinetic/Pharmacodynamic analysis of 20 patients across the first 7 cohorts (800 μg, 2400 μg, 0.1, 0.3, 1, 3, 10 mg/kg/Q1 wk dose) was performed, measuring PK and pharmacodynamics (FS118 engagement of LAG-3 or PD-L1 receptor in blood [soluble or T cell expressed]). PK analysis only was performed on one patient from the 20 mg/kg/Q1 wk dose cohort.

2.2.2.1 PK Analysis

For the PK analysis, serum FS118 levels were measured using a validated ligand binding assay utilising the GyroLab platform with biotinylated LAG-3 capture and Alexa Fluor® 647-labelled PD-L1 detection. Briefly, serum samples were diluted to a minimum required dilution (MRD) of 1:10 in Rexxip HN and added to plates which were then loaded, together with BioAffy 1000 CD(s), onto the Gyrolab XP workstation. FS118 was detected by fluorescence emission. The standard curve was regressed using a 5-parameter logistic curve with response as the weighting factor (1/y2) in the Gyrolab Evaluator application. The validated assay had an LLOQ of 100 ng/mL.

The results showed a dose linear increase in exposure with increasing dose across the 7 cohorts (800 μg, 2400 μg, 0.1, 0.3, 1, 3, 10 mg/kg/Q1 wk dose) (Cₘₐₓ, AUC). The Cₘₐₓ was approximately as predicted scaled from non-human primate but the clearance rate was higher than predicted (AUC 30% lower than predicted). There was a linear increase in exposure (Cₘₐₓ) and no accumulation of FS118 on weekly dosing up to a dose of 3 mg/kg. Some subjects in the 3, 10 and 20 mg/kg cohorts had measurable C_{trough} FS118 concentrations. Serum FS118 concentration at 7 days post-dose (prior to the next infusion) was below 100 ng/mL (LLOQ) for all patients at doses <1 mg/kg.

2.2.2.2 Soluble LAG-3

Serum total sLAG-3 was quantified using a validated enzyme linked immune-assay (ELISA), in the presence of a saturating amount of FS118. Briefly, sLAG-3 in serum samples was captured with plate-coated anti-LAG-3 monoclonal antibody (non-competitive binding). FS118 was added in vitro to saturate binding of sLAG-3. The captured sLAG-3:FS118 complexes were detected with a biotinylated anti-idiotype antibody against the FS118 Fcab domain engaged with sLAG-3 receptor, followed by addition of streptavidin conjugated-HRP and chromogen. The validated assay had an LLOQ of 0.675 ng/mL.

Analysis of soluble LAG-3 (sLAG-3) at the 1, 3 and 10 mg/kg/Q1 wk dose levels showed an approximately 10-fold increase in total sLAG-3 after the first dose of cycle 1 and cycle 2, with Cₘₐₓ peaking at 2-3 days post-dose.

The elevation of sLAG-3 levels confirms FS118 engagement of receptor. At the 1 mg/kg dose level, total sLAG-3 concentration returned to baseline values prior to the next dose (Cycle 1 Day 8; C1D8). At the 3 and 10 mg/kg/Q1 wk cohorts there was some evidence for an accumulation of total sLAG-3 (trough concentration) prior to the next dose. The extent and duration of increase in total soluble LAG-3 at the 3 mg/kg and 10 mg/kg doses suggested that saturation of soluble LAG-3 capture with FS118 had almost been achieved.

2.2.2.3 Soluble PD-L1

Plasma total soluble PD-L1 (sPD-L1) was quantified using a Meso-Scale Discovery immunoassay, in the presence of a saturating amount of FS118. The assay has an LLOQ of 0.458 ng/mL. The results showed early evidence of a transient increase in total soluble PD-L1 (sPD-L1) after each dose, although this was inconsistent across all patients and many patients had baseline concentrations below the level of quantification of the assay.

2.2.2.4 PD-L1 and LAG-3 Receptor Occupancy

PD-L1 and LAG-3 receptor occupancy was measured in whole blood T cells and monocytes. In brief, the method involved collection of whole blood in Cyto-Chex® tubes, stored at 4° C. until processing (100 μL per test). First, non-specific binding was blocked for 10 minutes at RT with 5 μL of Human BD Fc Block solution. Samples were then stained with one of three panels (Free Receptor, Total Receptor and FMO/Isotype) antibody cocktail (50 μL) and incubated for 30 minutes at RT followed by lysis of red blood cells and fixation with 900 μL of BD FACS Lysing Solution for 10 minutes at RT. Samples were then washed twice with 2% FBS before acquisition on the cytometer (LSR Fortessa). Receptor occupancy was calculated using the following formula (assumption: PD-L1 or LAG-3 target expression levels remain unchanged over the period of investigation):

$$T O ( \% ) = ( 1 - \frac{{D}_{t} - {C}_{t}}{{D}_{0} - {C}_{0}} ) \times 100$$

C—Median Fluorescence Intensity (MdFI) from isotype free target (competing) mAb

D—MdFI from free target (competing) mAb

C₀, D₀: MdFI values from pre-drug administration samples

Cₜ, Dₜ: MdFI from post-drug administration samples at a given time point

Overall, LAG-3 expression was 40- to 130-fold lower when compared with PD-L1 expression and the variability in estimated receptor occupancy was quite high (CV typically >50%). At 3 h after the first dose, mean PD-L1 receptor occupancy was 49 and 54% for the 3 and 10 mg/kg dose cohorts, respectively, and there was no obvious relationship between PD-L1 receptor occupancy and serum FS118 concentration. Similarly, at 3 h after the first dose, mean LAG-3 receptor occupancy was 23 and 32% for the 3 and 10 mg/kg dose cohorts, respectively, and there was no obvious relationship between LAG-3 receptor occupancy and serum FS118 concentration. Immediately prior to the second dose, PD-L1 and LAG-3 receptor occupancy was lower when compared with the 3 h post-dose time point.

Accumulation of total sLAG-3 and sPD-L1 and LAG-3 T cell receptor occupancy in the blood in some patients at C_{trough} levels for the 3, 10 and 20 mg/kg/Q1 wk cohorts where PK values were below the level of quantification or very low provide evidence of a sustained pharmacodynamic response at C_{trough} levels, thus surprisingly showing that FS118 exposure throughout the dosing interval is not needed for a pharmacodynamic effect in human patients.

The above results indicate that, in contrast to the results observed in mice with the mouse surrogate anti-LAG3/PD-L1 antibody, clearance of FS118 in humans is mainly LAG-3 mediated, more specifically membrane LAG-3 mediated, as clearance of sLAG-3 complexed with FS118 was found to be slower than the clearance of FS118.

2.2.3 Conclusions

The interim results available by May 2019 from the Phase I study demonstrated that FS118 was well-tolerated and that the maximum observed concentration (Cₘₐₓ) was in line with the Cₘₐₓ predicted from the cynomolgus monkey study but that the rate of clearance of FS118 was unexpectedly higher than predicted. This initially suggested that higher doses of FS118 in humans might be needed but despite the faster rate of clearance, a sustained pharmacodynamic response was observed at the lower doses tested which is indicative of therapeutic efficacy.

In particular, the results obtained showed that FS118 is capable of inducing a sustained increase in soluble LAG-3 (sLAG-3) levels at doses of 3 mg/kg, 10 mg/kg and 20 mg/kg administered once weekly, as well as sustained LAG-3 receptor occupancy. sLAG3 levels have been shown to be associated with therapeutic efficacy in mice. These interim results also suggested that sPD-L1 levels were increased following FS118 treatment.

2.3 Interim Data (August 2019)

2.3.1 Interim Clinical Data (August 2019)

By August 2019, a further 16 patients had been enrolled in the Phase I study. Thus, a total of 40 patients had been enrolled. Of these 40 patients, 16 were actively on treatment. The remaining 24 patients were discontinued from treatment: 11 patients due to iCPD, 3 patients due to non-related adverse events, 8 patients due to physician decision or clinical signs of progressive disease, and 2 patients due to other considerations.

Once weekly FS118 dosing was well tolerated up to 20 mg/kg and no dose limiting toxicity (DLT) was observed in Cycle 1 or subsequent cycles. Study-related Treatment Emergent Adverse Events (TEAEs) were observed in 62.5% of patients. None of these were deemed Treatment Emergent Serious Adverse Events (TE-SEAEs); 2 were deemed Grade 3 TEAEs based on elevated levels of transaminases. These 2 latter cases were reviewed by the safety committee for the trial who deemed that the elevated levels had no apparent clinical impact and classified the elevated levels as non-limiting toxicities. No apparent dose relationship between TEAEs and FS118 treatment was observed. No deaths or TE-SAEs that were observed were deemed to be related to FS118.

22 of 32 subjects in the cohorts receiving 3, 10 or 20 mg/kg had evaluable tumour scans. Of these 22 patients, 11 had some stable disease and 11 had progressive disease based on best overall response (BOR and iBOR). This represents a Disease Control Rate (DCR) of 34.4%.

For example, all of the patients in Table 7 (below) exhibited some stable disease during the ongoing trial and remained on study for at least 10 weeks.

TABLE 7
patients exhibiting some stable disease and
who remained on study for at least 10 weeks

| Dose | | Number of | Number |
|---|---|---|---|
| (mg/kg) | Tumour type | weeks on study | of scans |
| 1.0 | NSCLC | 24 | 3 |
| 3.0 | NSCLC** | 35 | 3 |
| | H&N | 28 | 3 |
| 10.0 | H&N | 17 | 2 |
| | Mesothelioma** | 27 | 3 |
| | CRC | 16 | 2 |
| | Cervical** | 18 | 2 |
| 20.0 | Thyroid** | 23 | 2 |
| | H&N** | 21 | 2 |

*NSCLC-non-small cell lung cancer;
H&N-head and neck cancer;
CRC-colorectal cancer
**study was on-going as at August 2019

In particular, subject 1004-0001 (suffering from NSCLC) had stable disease (RECIST 1.1 best response) and showed the best tumour reduction of 28.13 percent (change from baseline in sum of diameters (SoD)) which was observed at weeks 8 and 16 post FS118 dosing, decreasing slightly to 25% tumour reduction at week 24. Thus, this particular patient had a near Partial Response based on the measurement of their target lesions.

These results thus demonstrate that FS118 is capable of disease stabilization bearing in mind that the patient population included multiple different types of cancer, all patients had advanced malignancies, had failed on multiple alternative treatment regimens prior to entering the trial and some patients may have been too compromised to be capable of benefitting from treatment with FS118.

2.3.2 Interim Pharmacokinetic/Pharmacodynamic Data

By August 2019, the Pharmacokinetic/Pharmacodynamic analysis had been performed on up to 29 patients across 8 cohorts (800 μg, 2400 μg, 0.1, 0.3, 1, 3, 10 and 20 mg/kg/Q1 wk dose). As described in Example 2.2.2, free FS118 serum concentration was measured along with soluble LAG-3. Additionally, frequencies of proliferating (Ki67+) and total effector memory or central memory CD4+ and CD8+ T cells in the blood were measured, immune cell subsets were enumerated as well as LAG-3 and PD-L1 expression quantification in tumour tissues pre- and post-first dose of FS118.

2.3.2.1 PK Analysis

Following from the May 2019 results (see Example 2.2.2.1), free FS118 serum concentration levels were quantified in a further 9 patients including patients in the 20 mg/kg dosed weekly (Q1W) cohort. Free FS118 serum concentration levels were quantified using the validated ligand-binding assay described in Example 2.2.2.1.

Analysis of free FS118 serum PK profiles over the first week following the start of treatment cycle 1 and cycle 2 (3 weeks per cycle) showed a dose linear increase in exposure (Cₘₐₓ, AUC) across patient cohorts receiving either 800 μg, 2400 μg, 0.1, 0.3, 1, 3 or 10 mg/kg Q1W. PK analysis of samples from patients receiving 20 mg/kg was ongoing. Estimated Cₘₐₓ and AUC values were comparable between PK profiles from Cycle 1 and Cycle 2 within each patient cohort, which is indicative of low anti-drug antibody (ADA) response, low ADA-mediated accelerated clearance of FS118 or the absence thereof.

As seen in the May 2019 results, the Cₘₐₓ was approximately as predicted scaled from non-human primate but the clearance rate was higher than predicted (AUC 30% lower than predicted). Terminal clearance half-life (T_{1/2}) of free FS118 from 1-compartmental modelling, fitted on available phase I study data, is estimated to be 19.6 hours.

A week following dosing start (in cycle 1 and 2) and prior to the next FS118 dose, C_{trough} levels of free FS118 in the serum for patients receiving 1 mg/kg were below the lower limit of quantification (LLOQ) of the assay, which suggests the lack of free FS118 accumulation in the blood at <1 mg/kg Q1W dosing schedule. Some subjects in the 3, 10 and 20 mg/kg cohorts had quantifiable C_{trough} levels of free FS118 on day 7 post-dose in the range of approximately 0.1 to 10 μg/mL.

2.3.2.2 Soluble LAG-3

Following from the May 2019 results (see Example 2.2.2.2), serum total soluble LAG-3 (sLAG-3) levels were quantified in a further 9 patients including patients in the 20 mg/kg dosed weekly (Q1W) cohort. Serum total soluble LAG-3 (sLAG-3) levels were quantified using the validated ELISA described in Example 2.2.2.2.

Consistent with the May 2019 interim results, analysis showed dose-dependent increases in serum total sLAG-3. More specifically, patients receiving 1, 3, 10 or 20 mg/kg/Q1 wk dose levels showed an approximate 10- to 150-fold increase in total sLAG-3 after the first dose of cycle 1 and cycle 2, with time to maximal concentration (Tₘₐₓ) observed at approximately 2-3 days post-dose. At the 1 mg/kg dose level, total sLAG-3 concentration returned to baseline values prior to the next dose (C1D8). At the 3, 10 and 20 mg/kg/Q1 wk cohorts there was some evidence for an accumulation of total sLAG-3 (trough concentration) prior to the next dose; this is further evidenced by higher levels of sLAG-3 observed in cycle 2 compared to cycle 1. Further developing the May 2019 analysis, the extent and duration of increase in total soluble LAG-3 at the 10 and 20 mg/kg doses in particular suggests that saturation of soluble LAG-3 capture with FS118 at these dose levels has almost been achieved. However, additional patient numbers are required for the 20 mg/kg patient cohort in order to confirm this apparent observation.

Utilising this data, 1-compartment modelling estimated terminal clearance half-life (T_{1/2}) of 15.8 days for the sLAG-3:FS118 complex. The estimated terminal T_{1/2} of free sLAG-3 was 1.6 hours. A high correlation of population and individual analyses of FS118 PK and sLAG-3 data with Pharmacokinetic/Pharmacodynamic modelled data was observed. This confirms the absence of both ADA interference and ADA-mediated accelerated clearance of FS118.

In summary, this analysis showed that the dose-dependent increase of total sLAG-3 levels following FS118 dosing could be used as a pharmacodynamic marker of FS118 engagement of target LAG-3 receptors. This finding supports the proposed mechanism of action of FS118 whereby FS118 binding to LAG-3 receptors expressed on target cell surfaces leads to increased systemic soluble LAG-3 levels potentially through shedding of cell surface-expressed LAG-3.

2.3.2.3 Frequencies of Proliferating and Total Effector and Central Memory CD4+ and CD8+ T Cells in Blood

Frequencies of proliferating Ki67⁺ CD4⁺ and Ki67⁺ CD8⁺ effector memory and central memory T cells in the blood were monitored over time by flow cytometric analysis. In brief, whole blood was collected in Cyto-Chex® and stored under refrigeration until processing. 100 μL of each sample was used per test. First, non-specific binding was blocked with Human BD Fc Block solution. Samples were then stained with a surface antibody cocktail (50 μL) followed by lysis of red blood cells and fixation with BD FACS Lysing Solution. Washed cells were permeabilised with Fix/Perm Buffer, then washed twice with 1×Perm Buffer. Intracellular antibody cocktail (50 μL) was added and incubated for 30 min at 2-8° C. Cells were then washed twice with 2% FBS and transferred to TruCount tubes for acquisition on the cytometer (BD LSR).

The frequencies of CD4⁺ or CD8⁺ central memory T cells (defined by CD45⁺ CD3⁺ CD19^{neg} CD4⁺, or CD8⁺ respectively, CD45RA^{neg} CCR7^{pos} expression) or CD4⁺ or CD8⁺ effector memory T cells (defined by CD45⁺ CD3⁺ CD19^{neg} CD4⁺, or CD8⁺ respectively, CD45RA^{neg} CCR7^{neg}) were determined. In addition, frequencies of Ki67⁺ cells within the CD4⁺ or CD8⁺ effector or central memory T cell populations were determined. Analysis of available data from the 3, 10 and 20 mg/kg patient cohorts showed that FS118 was capable of inducing increases in frequencies of proliferating Ki67⁺ CD4⁺ and Ki67⁺ CD8⁺ effector memory and central memory T cells following dosing in cycle 1 relative to baseline measurements. The kinetic and transient nature of this peripheral pharmacodynamic response is indicative of T cell activation in-line with that observed with pre-clinical data.

In the same flow cytometric analysis described above, enumeration of immune cell subsets in the blood over time was performed. Initial data showed that FS118 dosing led to increased absolute numbers of CD3⁺, CD4⁺, CD8⁺ T-cells, and NK cells. The response kinetics were transient, similar to the pharmacodynamic effect observed on the frequencies of proliferating effector or central memory CD4⁺ and CD8⁺ T-cells in the blood. This was particularly observed in 4 patients suffering from mesothelioma, cervical cancer, anaplastic thyroid cancer and laryngeal cancer respectively. Taken together, the preliminary data suggest that FS118 can elicit a systemic immune activation response in patients.

2.3.2.4 PD-L1 and LAG-3 Expression in Tumour

Preclinical studies in mouse tumour models have previously shown that the mLAG-3/mPD-L1 bispecific antibody can induce LAG-3 suppression on LAG-3-expressing tumour infiltrating lymphocytes (TILs), whereas LAG-3 expression was increased when mice were treated with two antibody molecules comprising the same mLAG-3 and mPD-L1 binding sites as surrogate mLAG-3/mPD-L1 bispecific antibody (P2399 A LAG3/PD-L1 mAb² can overcome PD-L1-mediated compensatory upregulation of LAG-3 induced by single-agent checkpoint blockade, Faroudi et al., American Association for Cancer Research (AACR) Annual Meeting 2019, 29 March-3 Apr. 2019, Atlanta, Ga., USA).

To investigate this potential effect in the context of FS118, this being a bispecific hPD-L1/h LAG-3 antibody, paired tumour samples (N=4) were obtained from patients' pre-dose (ranging day −3 to −12) and post-dose (ranging from day 19 to 41). PD-L1 and LAG-3 expression in formalin-fixed and paraffin embedded (FFPE) tumour core needle biopsies were evaluated using an in vitro diagnostic (IVD) anti-PD-L1 (clone SP263) assay (Roche Diagnostics/Ventana Medical Systems) and a validated anti-LAG-3 (clone 17B4) immunohistochemistry (IHC) assay (Ventana BenchMark Ultra staining platform), respectively. For subsequent evaluation following IHC staining, selection criteria of 100 tumour cells and >25% tumour content were applied. Evaluation included determination of percent tumour positive score (% TPS) based on percentage and intensity of membranous anti-PD-L1 staining of tumour cells and quantification of PD-L1⁺ or LAG-3⁺ immune cells in up to 5 high power fields in the following compartments: intratumoural stroma, intraepithelial tumour component, or peritumoral area if applicable.

Overall, preliminary results at this point in the study showed no indication of compensatory upregulation of PD-L1 or LAG-3 expression in the tumour following FS118 dosing.

2.3.3 Conclusions

The interim results available by August 2019 support the conclusions observed in May 2019 (see Example 2.2.3). In short, FS118 was well-tolerated and the maximum observed concentration (Cₘₐₓ) was in line with the Cₘₐₓ predicted from the cynomolgus monkey study. The rate of clearance of FS118 was unexpectedly higher than predicted, but a sustained pharmacodynamic response was observed at the doses tested which is indicative of therapeutic efficacy. Indeed, by August 2019 11 patients were observed to have had some stable disease representing a Disease Control Rate (DCR) of 34.4%. These results demonstrate that FS118 is capable of disease stabilization bearing in mind that the patient population included multiple different types of cancer, all patients had advanced malignancies, had failed on multiple alternative treatment regimens prior to entering the trial and had no other treatment options available, and some patients may have been too compromised to be capable of benefitting from treatment with FS118.

In further support, the August 2019 results showed that FS118 is capable of inducing a sustained increase in soluble LAG-3 (sLAG-3) levels at doses of 3 mg/kg, 10 mg/kg and 20 mg/kg administered once weekly. sLAG3 levels have been shown to be associated with therapeutic efficacy in mice.

Furthermore, FS118 has been shown to induce a kinetic and transient peripheral pharmacodynamic response indicative of T cell activation in the 3, 10 and 20 mg/kg patient cohorts. In addition, increased proliferation of CD4⁺ and CD8⁺ central memory and effector T cells at C_{trough} levels provide further evidence for a sustained pharmacodynamic response at C_{trough} levels and there was no indication of compensatory upregulation of PD-L1 or LAG-3 expression in the tumour following FS118 dosing consistent with the hypothesised mechanism of action of FS118.

2.4 Interim Data (April 2020)

2.4.1 Interim Clinical Data (April 2020)

By April 2020, a further 3 patients had been enrolled into the study. Thus, a total of 43 patients were enrolled. Of these 43 patients, 2 patients were actively on treatment. The remaining 41 patients had completed/discontinued treatment: 14 patients due to iCPD, 4 patients due to non-related adverse events, 10 patients due to physician decision or clinical signs of progressive disease, and 10 patients due to other considerations. Of these 41 patients, 14 were in follow up and 27 had completed the study.

In respect of the further 3 patients enrolled into the study, once weekly dosing with FS118 IV administrations at a dose level of 20 mg/kg was well tolerated and no Dose Limiting Toxicities were reported to the sponsor.

In respect of all patients enrolled into the study, about 20% of the observed treatment emergent adverse effects were related to FS118 with the majority being of mild to moderate severity (Grade 1 or 2, Common Terminology Criteria for Adverse Events v4.3). Approximately 5% of FS118-related adverse events were categorised as Grade 3. No serious adverse events (SAEs) were reported with a relationship to FS118. SAEs are defined as any adverse event that results in death, is life threatening, requires hospitalisation, causes a disability, permanent damage to the patient's body or congenital anomalies/birth defects, requires intervention to prevent permanent impairment or damage or other serious events e.g. allergic bronchospasm. No deaths which occurred during the study were deemed to be related to FS118. In summary, no new safety risks were identified.

As of 25 Mar. 2020, 30/36 patients in 3, 10 or 20 mg/kg cohorts had evaluable tumour scans. 17 patients were recorded as having stable disease (SD) and 13 having progressive disease (PD) as their best response to treatment with FS118 (BOR and iBOR). This represents a Disease Control Rate (DCR) of 47.2%, corresponding to an increase of 12.8% from August 2019. The 17 patients recorded as having stable disease are listed in Table 8 (below).

TABLE 8
17 patients exhibiting stable disease as BOR/iBOR when
administered FS118 at a dose of 3, 10 or 20 mg/kg once weekly

| Dose | | Number of |
|---|---|---|
| (mg/kg) | Tumour type | weeks on study |
| 3 | Head & Neck | 26 |
| | NSCLC | 44 |
| | Ovarian | 11 |
| 10 | Cervical Cancer | 28 |
| | CRC | 15 |
| | CUP | 6 |
| | Head & Neck | 15 |
| | Melanoma | 15 |
| | Mesothelioma | 35 |
| | NSCLC | 12 |
| 20 | Anaplastic Thyroid | 55** |
| | Head & Neck | 11 |
| | | 12 |
| | | 27 |
| | Ovarian | 21 |
| | Prostate | 10 |
| | Leiomyosarcoma | 32** |

NSCLC-non-small cell lung cancer;
CRC-colorectal cancer;
CUP-Cancer of Unknown Primary
**study was on-going as at Mar. 25, 2020

Of the two remaining patients on the Phase I study (both receiving a once weekly 20 mg/kg dose), 1 patient had leimyosarcoma (soft-tissue sarcoma) and had been on study for 32 weeks as at 25 Mar. 2020; the other patient had anaplastic thyroid cancer (ATC) and had remarkably been on study for >1 year (55 weeks) as at 25 Mar. 2020.

2.4.2 Conclusions

These results continue to demonstrate that FS118 exhibits favourable tolerability when administered over an extended period and, more importantly, that treatment with FS118 at dosages within the range 1-20 mg/kg can result in long term disease stabilization (multiple patients >18 weeks completed with SD as BOR/iBOR; 1 patient >1 year and remaining on-study). This is particularly significant as the patient population for the trial included multiple different types of cancer, all patients had advanced malignancies, had failed on multiple alternative treatment regimens prior to entering the trial and some patients may have been too compromised to be capable of benefitting from treatment with FS118. Despite this challenging patient population, FS118 was able to achieve a Disease Control Rate (DCR) of 47.2%, corresponding to an increase of 12.8% from August 2019. This increase further demonstrates that doses in the range 3-20 mg/kg of FS118 are able to achieve disease stabilization.

### Example 3: Selecting Patients More Likely to Respond to FS118 Based on Resistance to Prior Anti-PD-1 or Anti-PD-L1 Therapy

3.1 Background

All patients included in the ongoing FS118 trial had previously progressed while receiving or after receiving PD-1/PD-L1 containing therapy.

Initial results (August 2019) demonstrated that FS118 is capable of disease stabilization in some patients with a disease control rate (DCR) of 34.4% (see Example 2.3.1), rising to 47.2% by April 2020 (see Example 2.4.1). The inventors hypothesized that FS118 may be providing benefit to these patients due to the additional benefit provided by LAG-3 inhibition in combination with PD-L1 inhibition (dual checkpoint inhibition) or by novel biology provided by the bispecific targeting of PD-L1 and LAG-3 (WO2017220569A1). It was not expected that patients would achieve clinical benefit with re-treatment with anti-PD-1 or anti-PD-L1 containing regimens alone (Fujita et al., Anticancer Res. (2019); Fujita et al., Thoracic Cancer (2019); Martini et al., J. Immunotherapy Cancer (2017)).

One of the mechanisms for resistance to PD-1/PD-L1 blockade may be up-regulation of signaling receptors that can impair T cell functionality (Nowicki et al., The Cancer Journal (2018)); this class of receptors includes LAG-3. This mechanism of resistance is thought to be a form of acquired resistance where T cells initially respond but subsequently become exhausted leading to a loss of T cell function. This contrasts with primary resistance where patients fail to respond to initial therapy.

The inventors therefore hypothesized that FS118 may be most likely to provide clinical benefit to patients with acquired resistance to anti-PD-1/PD-L1 therapy and performed analysis to define specific criteria that could be used to select patients for treatment with FS118. To define these criteria, sub-groups based on each patient's previous treatment history with anti-PD-1/PD-L1 therapies were defined (Best Overall Response (BOR) to these therapies and the number of months of treatment with these therapies). Clinical benefit derived from FS118 was based on the number of weeks that each patient received FS118 treatment, termed “FS118 weeks completed”.

3.2 Methodology

Of the patients enrolled in the Phase I trial by December 2019, the treatment history with anti-PD-1 or anti-PD-L1 therapies was known for 43 patients. These prior anti-PD-1 or anti-PD-L1 therapies included treatment with nivolumab, pembrolizumab, avelumab, durvalumab, atezolizumab, Cemiplimab, MSB-2311 or KN035, either alone or in combination with another agent (e.g. a chemotherapeutic or immunotherapeutic (e.g. anti-CTLA-4)). It was not necessarily the case that the prior anti-PD-1 or anti-PD-L1 therapy immediately preceded treatment with FS118, but rather the prior anti-PD-1 or anti-PD-L1 therapy could have occurred at any time during the patient's treatment history for the cancer in question.

Initially, 6 sub-groups based on treatment history were defined as follows:
PD (Progressive Disease (by RECIST 1.1; Eisenhauer et al., 2009) as BOR on any previous anti-PD-1 or anti-PD-L1 containing therapy regardless of treatment duration);
SD (Stable Disease (by RECIST 1.1) as BOR and a treatment duration of 3 months or less with any previous anti-PD-1 or anti-PD-L1 containing therapy (presented as “0-3 months”);
SD (Stable Disease (by RECIST 1.1) as BOR and a treatment duration of more than 3 months but less than 6 months with any previous anti-PD-1 or anti-PD-L1 containing therapy (presented as “3-6 months”)
SD (Stable Disease (by RECIST 1.1) as BOR and a treatment duration of 6 months or longer with any previous anti-PD-1 or anti-PD-L1 containing therapy (presented as “6+ months”) and
PR (Partial Response (by RECIST 1.1) as the BOR to any previous anti-PD-1 or anti-PD-L1 containing therapy).
Unknown RECIST criteria (BOR) to prior anti-PD-1/PD-L1 therapy (“UNK”), regardless of treatment duration with the previous anti-PD-1 or anti-PD-L1 containing therapy.

None of the patients assessed in this study had a CR (Complete Response) to a prior anti-PD-1 or anti-PD-L1 containing therapy and therefore no sub-group was established for CR patients.

Patients in each sub-group defined above were first plotted against the number of weeks that each patient received FS118 treatment “FS118 weeks completed”.

Following this initial analysis, the following definitions of Primary and Acquired resistance were derived:
“Primary resistance” defined as a combination of the PD sub-group and the SD 0-3 months sub-group.
“Acquired resistance” defined as a combination of the SD 3-6 months, SD 6⁺ months and PR sub-groups.
Unknown (BOR to prior anti-PD-1/PD-L1 therapy not known)

If patients who had a CR to a prior anti-PD-1 or anti-PD-L1 containing therapy had been present in the study, they would also have been categorised in the Acquired Resistance group, based on them having achieved significant clinical benefit from their prior therapy before progression, akin to the SD 3-6 months, SD 6⁺ months and PR sub-groups. This is because anti-PD-1 and anti-PD-L1 therapies can result in complete responses, and some of these patients do subsequently develop resistance mechanisms and progressive disease. It is expected that the mechanisms of resistance in patients achieving a complete response following anti-PD-1 or anti-PD-L1 are similar to those patients who achieve a partial response.

Each of the Primary resistance, Acquired resistance and Unknown sub-groups were plotted against “FS118 weeks completed”.

For the plots, “FS118 weeks completed” data was accurate through to 25 Mar. 2020. These data were from an ongoing trial. All plots were produced using R version 3.6.1 (www.crans.r-project.org) with the package ‘ggplot’ and statistical analyses using the non-parametric Wilcoxon Rank Test calculation were performed using the same version of R.

3.3 Results

Using patient data accurate through to December 2019, analysis of the 6 initial sub-groups defined based on prior response to anti-PD-1/PD-L1 containing therapies (PD, SD 0-3m, SD 3-6m, SD 6m+, PR, UNK) did not show a significant difference (Wilcoxon Rank Sum Test p>0.05) between these groups with respect to number of weeks FS118 treatment that had been completed by these patients at the time of analysis. However, a trend was observed that patients with a best overall response (BOR) to prior anti-PD-1/PD-L1-containing treatment of stable disease (SD) who had received this anti-PD-1 or anti-PD-L1 treatment for greater than 3 months (including partial responders) showed longer duration of response times to FS118 when compared to the PD and SD 0-3m sub-groups.

Based on this surprising observation, the 6 initial sub-groups were subsequently parsed into two groups based on patients' responses to prior treatment with anti-PD-1/PD-L1 containing therapies. The first group contained the PD and SD 0-3 months sub-groups and was termed “Primary Resistant”, based on the fact that these patients derived no significant clinical benefit from the prior anti-PD-1/PD-L1 therapy. The second group contained the SD 3-6 months, SD 6 months+ and PR sub-groups and was termed “Acquired Resistant” based on patients having derived clinical benefit on prior anti-PD-1/PD-L1 therapy for more than 3 months before subsequently experiencing progressive disease.

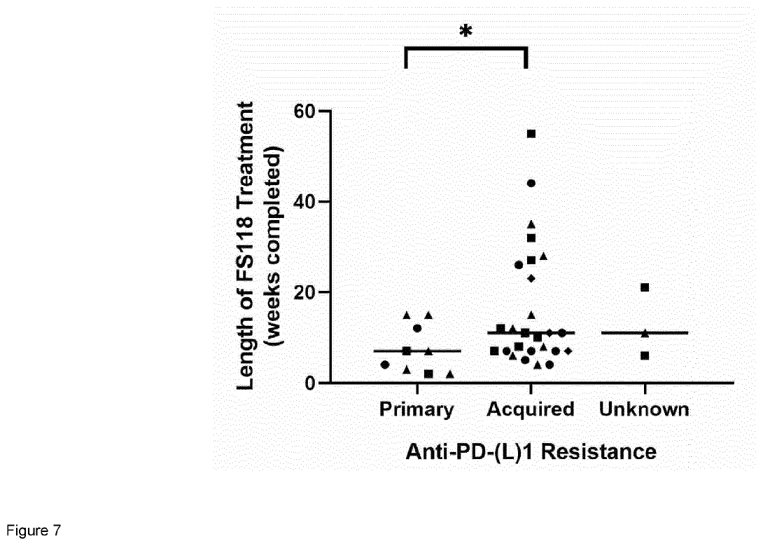

When grouping patients into Primary and Acquired resistance groups, a striking difference was observed between these two patient groups when each was compared against the number of weeks that patients in the respective group remained on FS118 treatment (Mann-Whitney-Wilcoxon Test p=0.059). More specifically, patients in the Acquired resistance group were observed to be more likely to respond to, and derive benefit from, treatment with FS118. In particular, all patients completing 18 weeks or more on FS118 treatment were from the Acquired resistance group, with the exception of one patient for whom the BOR on their prior anti-PD-1 therapy was unknown. However, it was known for this latter patient with unknown BOR that they had stayed on the prior anti-PD-1 therapy for more than one year and thus it is suspected that this patient would have had a BOR that would classify as having acquired resistance. None of the Primary resistant patients were able to stay on study for more than 17 weeks. These observations were further supported by additional clinical data available at 25 Mar. 2020 which, due to patients in the Acquired resistance group continuing to remain on treatment, found that the statistical significance of the difference observed between the Primary and Acquired resistant patient groups was improved (Mann-Whitney-Wilcoxon Test p=0.048, FIG. 7).

Moreover, it is important to note that whilst this analysis is from an ongoing study, none of the Primary resistant patients have remained on study.

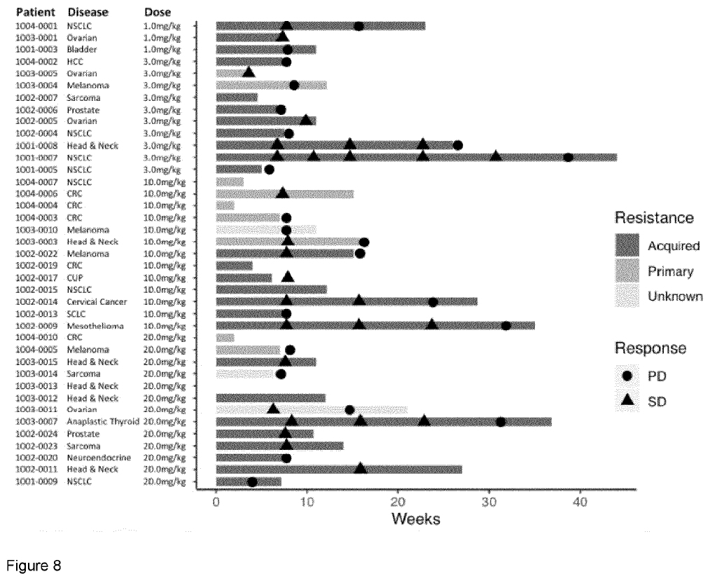

By December 2019, 39 patients had evaluable tumour scans whilst on FS118 treatment. These patients are shown in FIG. 8 with an indication as to whether each patient has an Acquired or Primary resistant phenotype. All patients with more than 18 weeks of FS118 treatment completed (which had an Acquired resistance phenotype) had at least one measurement of stable disease (FIG. 8), although stable disease was observed in both Acquired and Primary resistant populations.

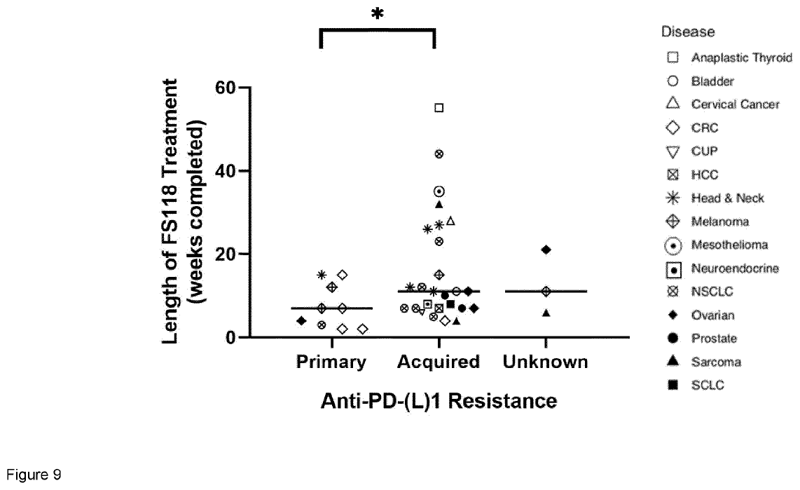

The phenomenon observed for Acquired resistance patients in terms of likelihood of response to FS118 treatment appeared to be independent of any particular dose level (FIG. 7) or clinical indication (FIGS. 8 and 9).

3.4 Conclusions

In summary, patients with Acquired resistance (defined as having a BOR of SD, PR or CR and therefore having derived some clinical benefit while on prior anti-PD-1/PD-L1 therapy for a treatment duration of more than 3 months before subsequently experiencing progressive disease) have surprisingly been found to be more likely to positively respond to FS118 treatment for longer than patients with Primary resistance (defined as patients deriving no clinical benefit or some clinical benefit from prior anti-PD-1/PD-L1 therapy lasting 3 months or less). This is particularly significant because re-treatment of patients with a PD-(L)1 antibody after disease progression on a prior PD-(L)1 containing treatment regimens is not recommended and historically patients derive little benefit (Fujita et al., Anticancer Res. 2019; Fujita et al., Thoracic Cancer, 2019; Martini et al., J. Immunotherapy Cancer, 2017). Thus, the present inventors have identified a threshold to select for patients more likely to respond to FS118 treatment. This threshold appears to be independent of FS118 dose or cancer type.

As a side note, the present inventors then identified three clinical studies underway investigating IgG4 monoclonal antibody BI-754111 (anti-LAG-3) in combination with BI-754091 (anti-PD-1 mAb): NCT03697304, NCT03780725 and NCT03156114. These studies refer to the use of patient cohorts who exhibit secondary resistance (acquired resistance) to prior anti-PD-1 or anti-PD-L1 based therapy. None of the information publicly available in relation to these clinical studies indicates why these cohorts have been selected, how the definitions of secondary resistance were derived nor offer any suggestion or data that demonstrate an improved response in the secondary resistance patient cohorts relative to a primary resistant patient population. These clinical studies therefore offer no assistance in the context of FS118 nor appear relevant for the present studies.

### Example 4: PD-L1 Expression as a Marker to Select Patients for Treatment with FS118 Based on Resistance to Prior Anti-PD-1 or Anti-PD-L1 Therapy

4.1 Background

As all patients taking part in the ongoing FS118 trial had received prior treatment with anti-PD-1 or anti-PD-L1 therapies, and it has been shown by Faroudi et al. (American Association for Cancer Research (AACR) Annual Meeting 2019, 29 March-3 Apr. 2019, Atlanta, Ga., USA) that targeting these pathways can change the levels of checkpoint receptors, we sought to determine the expression levels of PD-L1 and LAG-3 before treatment with FS118 (“baseline”), and determine if any correlation existed between these expression levels and time on treatment with FS118. For this analysis, patients were grouped as either having “Acquired” or “Primary” resistance to their prior treatment with anti-PD-1 or PD-L1 therapy, as defined in Example 3.

4.2 Methods

PD-L1 expression were measured in biopsies taken from patients before treatment with FS118 (“baseline”). In order for the biopsy to be eligible for analysis, tumour cell content had to be 25% or more and ≥100 tumour cells needed to be present. Tumour samples were formalin-fixed and paraffin embedded (FFPE), stained and evaluated as described in Example 2.3.2.4. The PD-L1 percent tumour positive score (% TPS) was calculated as the percentage of tumour cells in the biopsy sample showing positive staining for PD-L1. % TPS was measured for all available samples, which were: 13 subjects with Acquired resistance and 4 subjects with Primary resistance.

4.3 Results

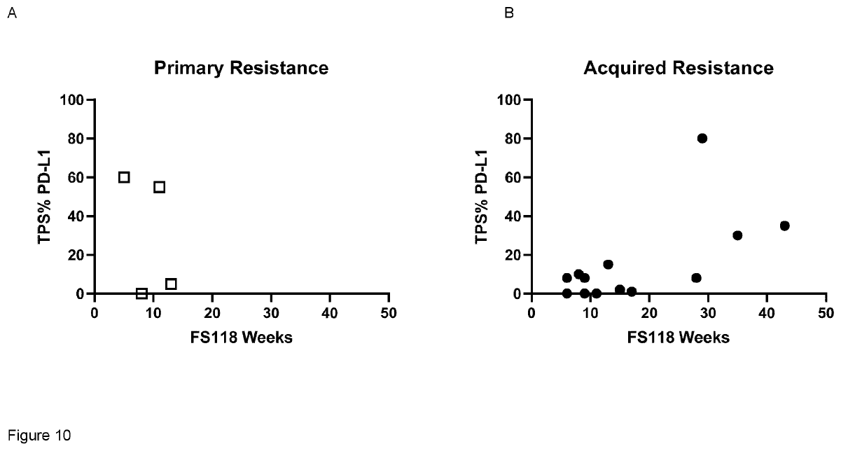

When comparing PD-L1% TPS at baseline and the number of weeks treatment with FS118, a positive correlation was observed for the Acquired resistance group (One-tailed Spearman Correlation Coefficient r=0.57, p=0.022). Conversely, no correlation between PD-L1% TPS and FS118 treatment time was found for the Primary resistance patient group (One-tailed Spearman Correlation Coefficient r=−0.40, p=0.37). Furthermore, within the Acquired resistance group, three patients were treated with FS118 for 30 weeks or more evidencing disease control by FS118. These three patients also had the highest PD-L1% TPS within the group (see FIG. 10). Using the positive correlation for the Acquired resistance group, a prognostic threshold that could be used to select patients who are more likely to respond to treatment with FS118 was determined. This was done by plotting the correlation trend line and, via interpolation, using this to determine the PD-L1% TPS score that correlated with 18 weeks of FS118 treatment. 18 weeks was chosen because remaining on 18 weeks treatment or more was observed in the Acquired resistance group, but not in the Primary resistance group, and therefore deemed indicative of clinical benefit with FS118. The PD-L1% TPS score determined in this way was 15%.

4.4 Conclusions

Overall, these results demonstrate that expression of PD-L1 by the tumour (PD-L1% TPS) in Acquired resistant patients positively correlates with the longevity of disease control achieved by FS118. The 3 patients with the highest PD-L1% TPS in the Acquired resistance group all had a long duration of disease control by FS118 (30 weeks or more on FS118 treatment). Using the positive correlation for the Acquired resistance group, a PD-L1% TPS of 15% was established as a prognostic threshold to select patients in the Acquired resistance group particularly likely to exhibit a sustained response to treatment with FS118.

### Example 5: Effect of FS118 on the Immune Response in Acquired and Primary Resistant Patients

5.1 Background

Following the observation in Example 3 that Acquired resistant patients are more likely to remain on FS118 treatment for longer than Primary resistant patients, the inventors sought to determine whether there was a difference in the pharmacological response to FS118 between these two groups. Patients with Primary resistance may fail prior anti-PD-1/PD-L1 therapy due to inadequate T cell function as a result of suppressive factors in the tumour or a lack of recognition of the tumour by the immune system (Nowicki et al., 2018). Patients with Acquired resistance may initially have a T cell response but are believed to have a loss of T cell function which could result from multiple mechanisms including up-regulation of LAG-3. Activation of T cells by FS118 has been demonstrated to be a mechanism of action of FS118 in vitro (WO2017220569A1). Therefore, the inventors hypothesised that the ability of the patient's immune system to respond to FS118 may depend on their response to prior anti-PD-1/PD-L1 therapy and that the ability of FS118 to potentiate an immune response may be important in FS118 providing clinical benefit.

5.2 Methodology

During the course of treatment with FS118, the effect of FS118 on the peripheral immune cell count in the bloodstream of 35 patients on the trial was assessed (24 patients with Acquired resistance, 8 patients with Primary resistance and 3 unknowns—as defined in Example 3). Blood samples were obtained from patients and absolute cell counts of CD3⁺ lymphocytes, CD4⁺ T cells, CD8⁺ T cells, B cells, and NK cells (TBNK cell counts) of whole blood immune cells were performed by Caprion Biosciences (Caprion Biosciences, Inc., Montreal, Quebec, Canada). In short, the collected blood was stored in Cyto-Chex® BCT tubes at 4° C. until processing. Then, 100 μL of whole blood was used to stain with a pre-defined TBNK panel in single replicates by Caprion. Following staining, samples were acquired within 24 h on a BD LSR flow cytometer and quantitated using FlowJo Software. Absolute cell counts were measured at several time points both before FS118 treatment (termed “baseline”) and during FS118 treatment.

In the subsequent data analysis of absolute cell counts, only patients that received a dose greater than or equal to 1 mg/kg of FS118 were taken into account.

The percentage change of cell count from baseline per cell type was calculated as follows:

Percentage change from baseline=[(cell count_{at treatment day}−cell count_{at baseline})/cell count_{at baseline}]*100

For each cell type, the percentage change from baseline was then plotted against time on FS118 treatment. The immune response profile based on immune cell counts was calculated for each patient individually.

Patients were categorised into “Primary” or “Acquired” resistance as defined in Example 3.

5.3 Results

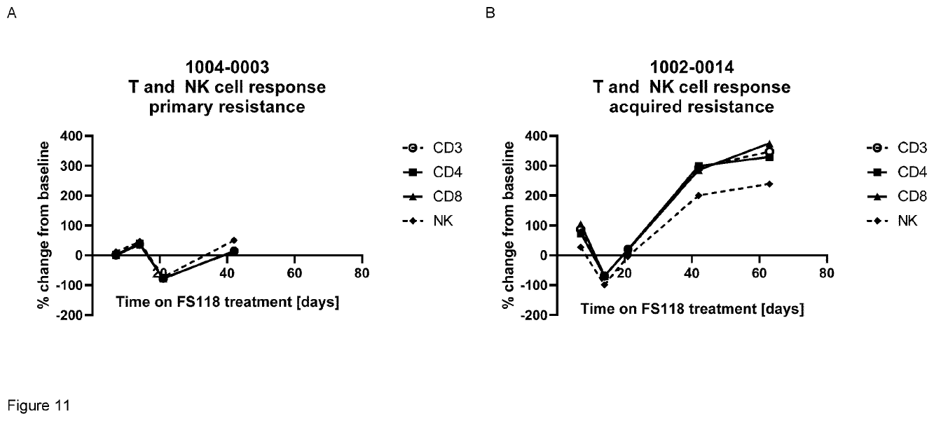

FIG. 11 shows the percentage change from baseline for two representative patients: Patient 1004-0003 as a representative example of an immune cell response profile for a patient with “Primary resistance” and patient 1002-0014 as a representative example of an immune cell response of a patient with “Acquired resistance”. Patients with Acquired resistance showed a trend toward increased numbers of CD3⁺ lymphocytes, CD4⁺ T cells, CD8⁺ T cell and NK cells than patients with Primary resistance (based on percentage change from baseline for these cell sub-sets).

In addition, the highest fold change in CD3⁺ lymphocytes from baseline observed during the course of FS118 treatment was plotted against time on FS118 treatment for each patient. This was done for both Primary and Acquired resistant groups. The magnitude of CD3+ lymphocyte response measured by the fold-change of immune cell counts compared to baseline was found to significantly positively correlate with the duration of FS118 treatment in the Acquired resistant group (One-tailed Spearman Correlation Coefficient r=0.45, p=0.025), however this was not significant in the Primary resistant group (One-tailed Spearman Correlation Coefficient r=0.52, p=0.098).

5.4 Conclusions

These data demonstrate that the increases in T and NK cells observed in patient blood, as well as fold-change increases in CD3⁺ lymphocytes, are a consequence of treatment with FS118 and indicate that the immune systems of patients with Acquired resistance are more able to raise an immune response with FS118 treatment. Therefore, Acquired resistance as defined herein can be used as a threshold to select patients more likely to respond to FS118.

### Example 6: Dose Recommendation for Phase I Expansion and/or Phase II Trials Based on FIH Data and Modelling

6.1 Overview

To guide dose selection for future clinical studies, multiple parameters of the FIH Phase I trial data (described in Examples 2-5) were collected and analysed. Per dose tested in the FIH Phase I trial, these parameters included the presence of anti-drug antibodies (ADA) and Treatment Emergent Adverse Events (TEAE), as well as efficacy as assessed by time on treatment, tumour growth rate, tumour size by sum of diameter and number of responders. Simulations of LAG3:FS118:PD-L1 receptor trimeric complex formation, total sLAG3 and total sPD-L1 profiles in serum were also performed. While no differences were observed in most parameters when comparing between doses, ADA levels, efficacy as assessed by the number of responders and the simulation of trimeric complex formation showed sufficient differences between doses to enable a preferred dose to be recommended for further studies.

6.2 ADA Analysis of FIH Serum Samples

Administration of protein therapeutics such as FS118 can induce anti-drug antibodies (ADA), which can have an impact on their Pharmacokinetic/Pharmacodynamic characteristics. The detection and characterisation of ADA against FS118 in human serum samples in the FIH study were performed in support of clinical study. The FS118 ADA assay was developed at BioAgilytix Labs. Briefly, a bridging assay using the electrochemiluminescence (ECL) MSD platform, similar to that described in Example 1.2.2, was used to measure antibodies that bind to FS118 in human serum; biotinylated FS118 was used to capture ADA which were then detected using FS118 tagged with MSD TAG-NHS-ester (MSD #R91BN). ADA levels (ECL signal) from patient serum samples were measured, normalised to negative control consisting of pooled untreated human serum, and grouped by FS118 dose (Table 9).

TABLE 9
normalised ADA levels in patients from the FIH Phase I trial

| | ADA level | | |
|---|---|---|---|
| Dose | (normalised ECL | | |
| (mg/kg) | signal) | SEM | p value |
| 3 | 8908 | 6375 | Not applicable |
| 10 | 120.0 | 81.24 | 0.048 |
| | | | (vs 3 mg/kg; |
| | | | Mann Whitney test) |
| 20 | 38.13 | 22.77 | 0.006 |
| | | | (vs 3 mg/kg; |
| | | | Mann Whitney test) |

The 3 mg/kg once weekly dosing group had significantly higher levels of ADA when compared against the 10 mg/kg or 20 mg/kg once weekly dosing regimens (p≤0.05, Mann Whitney test). Higher ADA levels at a lower drug dose, which may also be referred to as higher doses “dosing through” the ADA response, is a commonly observed phenomenon (Chirmule, 2012). Despite the difference in the levels of ADA, no apparent dose relationship between TEAEs and FS118 treatment was observed (see Example 2.3.1). To minimise the possible impact of ADA on immunogenicity, Pharmacokinetic/Pharmacodynamic characteristics and toxicity, 10 mg/kg and 20 mg/kg once weekly dosing regimens would be preferred for future studies.

6.3 Bayesian Analysis of Efficacy Data from the FIH Phase I Trial

Using the FIH BOR/iBOR efficacy data collected in response to treatment with FS118, Bayesian analysis was used to predict the frequency of patients within each of the 3, 10 and 20 mg/kg once weekly dosing groups that will exhibit stable disease in future trials.

The occurrence of stable disease as BOR/iBOR in patients from the FIH Phase I trial was calculated for patients in the 3 mg/kg, 10 mg/kg and 20 mg/kg once weekly dose groups. This data was used to estimate the probability of a patient exhibiting stable disease at each dose level in future trials as shown in Table 10 below:

TABLE 10
Estimated probability of a patient exhibiting stable
disease at different dose levels in future trials with FS118

| FS118 dose | | No. of | |
|---|---|---|---|
| administered | Total no. | patients with | |
| (mg/kg once | of patients | stable disease as | Estimated |
| weekly) | dosed | BOR/iBOR | probability |
| 3 | 8 | 3 | 0.375 |
| 10 | 11 | 7 | 0.636 |
| 20 | 11 | 7 | 0.636 |

For example, assuming enrolment of 24 patients in a future trial (e.g. Phase I expansion or Phase II) and utilising the estimated probabilities shown in Table 10 factored with 90% confidence intervals, it is estimated that the number of responders (i.e. patients exhibiting at least stable disease as BOR/iBOR) per dose of FS118 would be as follows:

3 mg/kg once weekly: 4-14 responders

10 mg/kg once weekly: 11-19 responders

20 mg/kg once weekly: 11-19 responders

As shown above, both the 10 mg/kg once weekly and 20 mg/kg once weekly doses are predicted to achieve the best response outcome by eliciting stable disease in the highest proportion of patients. Thus, either of these doses would be preferred for future trials based on this Bayesian analysis.

6.4 Trimeric LAG3:FS118:PD-L1 Receptor Complex Formation as a Pharmacodynamic Marker

Activation of T cells by FS118 has been demonstrated to be a mechanism of action of FS118 in vitro (WO2017220569A1). Therapeutic efficacy of FS118 in the tumour was hypothesised to be as a result of tumour-specific T cells being activated in the tumour microenvironment as a consequence of FS118 binding to LAG-3 and PD-L1 simultaneously and inhibiting the immunosuppressive signals otherwise generated by LAG-3 and PD-L1 signalling. Using serum-derived data from the FIH Phase I trial, trimeric complex formation was simulated in both the serum and in the tumour microenvironment and used as a pharmacodynamic marker for dose regimen selection, in particular to select between the 10 mg/kg and 20 mg/kg once weekly dose regimens.

From the Pharmacokinetic/Pharmacodynamic data collected in the FIH study, the median free FS118, total sLAG3 and total sPD-L1 serum concentration profiles by dose in weeks 1 and 4, were derived. These data provided the basis to predict that the higher the FS118 dose, the higher the free FS118 concentrations in both serum and in the tumour microenvironment. From these data, a population model for free FS118, total sLAG-3 and total sPD-L1 serum concentrations in patients with advanced solid tumours was then developed. This model was used to run simulations linking dose regimen with the formation of the trimeric complex in the tumour in order to inform the selection of a dose regimen for future trials. A stepwise approach was used for model development (Table 11). First, free FS118 serum concentration was modelled with a one-compartmental PK model and linear elimination. Then, total sLAG-3 and total sPD-L1 serum concentrations were added and fitted with a binding model. Simulations of binding to FS118 and slower elimination of the FS118:sLAG-3 and FS118:sPD-L1 complexes compared to free sLAG-3 and free sPD-L1 were able to explain the observed increase in total sLAG-3 and total sPD-L1 serum concentration upon FS118 treatment seen in patients. Initially, the in vitro measured equilibrium dissociation constants were used for the respective complexes, but a model with estimated equilibrium dissociation constants was able to better describe the observed profiles. The fit of the free FS118 serum concentration profiles was not modified by the addition of the binding and elimination of sLAG-3 and sPD-L1. sLAG-3 and sPD-L1 were assumed to be constantly produced from an unspecified source.

At this point, the model was able to describe the observed free FS118, total sLAG-3 and total sPD-L1 serum concentrations. In order to link the FS118 dose regimen with efficacy, binding to cell surface LAG-3 and PD-L1 receptors in both serum and the tumour microenvironment were added to the model after parameter estimation to determine the trimeric FS118:LAG-3:PD-L1 complex in serum and the tumour microenvironment.

For modelling of the tumour microenvironment, simplistic assumptions were made. Firstly, free FS118 tumour concentration was assumed to always be a fraction of the free FS118 serum concentration (represented as a biodistribution coefficient) and that there was instant equilibration between the serum and the tumour. With the assumption that the tumour mass was low and that it will not affect the systemic FS118 concentrations, no mass flow of FS118 from serum to tumour was modelled. Thus, free FS118 concentration in tumour was estimated with a biodistribution coefficient (BC) as [FS118]ₜᵤₘₒᵤᵣ=BC [FS118]ₛₑᵣᵤₘ. Tumour concentrations of LAG-3 and PD-L1 were assumed to be the same as serum concentrations of LAG-3 and PD-L1 receptors which were assumed to be constant. Binding to the cell surface receptors was modelled using the same equilibrium dissociation constants estimated for the binding to the soluble targets.

TABLE 11
Main steps of model development

| Step | Action | Result |
|---|---|---|
| 1. | 1-compartment model with linear | Good fit: model taken forward |
| Fit the observed free | elimination | |
| FS118 serum | Elimination through internalization by | Unable to describe the observations: |
| concentration | target receptor | model dropped |
| 2. | Estimated K_{D}s vs fixed to in vitro | Better fit with estimated K_{D}s: model |
| Fit the observed total | measured values for sLAG3 and sPD- | with estimated K_{D}s taken forward |
| SLAG3 and sPD-LI | L1 binding | Better fit of the total sLAG3 |
| serum concentrations | Constant sLAG3 production vs FS118- | observations with the constant |
| | depdendent sLAG3 production based | production rate: model with constant |
| | on in vitro LAG3 shedding data | sLAG3 production rate taken forward |
| 3. | Add LAG3 and PD-L1 cell surface | No change to the fit of the observed |
| Additions to the model for | receptors | free FS118, total sLAG3 and total |
| simulations | Add trimeric complexes | sPD-L1 observations |
| | Add tumour compartment with a BC | No change to the fit of the observed |
| | | free FS118, total sLAG3 and total |
| | | sPD-L1 observations |
| | | By model design, no impact on the |
| | | serum concentrations |

The following dose regimens were simulated: (i) 1, 3, 10 or 20 mg/kg administered once weekly as a 1-hour IV infusion, or (ii) 3, 10 or 20 mg/kg administered once every two weeks as a 1-hour IV infusion. Simulations were done in R 3.6.0 (R Development Core Team 2008) using the mlxR 4.0.0 library. The simulations used the individual parameter estimates by Monolix (mode of conditional distributions) from the FIH Phase I trial patients. The means of these individual predicted profiles were then plotted. It was investigated which of the simulated dose regimens produced the highest trimeric complex concentration (LAG3:FS118:PD-L1) in serum and in the tumour.

The mean of simulated individual cell surface trimeric complex (LAG3:FS118:PD-L1), as a percentage of total LAG-3 receptors, in serum and tumour for different BCs, using the individual estimates from the Phase I trial patients was obtained and plotted. The simulations revealed that higher free FS118 concentrations resulted in lower trimeric LAG3:FS118:PD-L1 complex concentration, favouring the dimeric FS118:LAG3 and FS118:PD-L1 complexes. It had also been shown using the data collected in the FIH study that the higher the FS118 dose, the higher the free FS118 concentration. The optimal free FS118 concentration range was approximately 0.1-1 μg/mL. For a BC of 10%, assumed to most closely mimic the tumour microenvironment in vivo, 10 mg/kg once-weekly dosing had a higher trimeric complex concentration than the 20 mg/kg once-weekly dosing by virtue of being more likely to generate a free FS118 concentration in the range 0.1-1 μg/mL.

This also means that too high doses and/or too frequent dosing would reduce the trimeric complex concentration and therefore reduce T cell activation induced by simultaneous binding of FS118 to LAG-3 and PD-L1, and therefore result in a reduced effect on inhibition of tumours.

6.5 Conclusions

Multiple elements of the FIH Phase I trial data were analysed and used to guide dose selection for future trials. Firstly, the analysis of ADA at different FS118 doses showed that higher levels of ADA were detected in 3 mg/kg once weekly dosing compared to 10 mg/kg or 20 mg/kg once weekly dosing. To minimise potential immunogenicity and toxicity, the 10 mg/kg once weekly and 20 mg/kg once weekly regimens would be preferred over 3 mg/kg once weekly. Secondly, Bayesian analysis of the Phase I BOR data estimated that there would be a greater likelihood of patients exhibiting SD as BOR/iBOR if administered 10 mg/kg once weekly or 20 mg/kg once weekly regimens than a 3 mg/kg once weekly regimen. Finally, the Pharmacokinetic/Pharmacodynamic modelling and simulations of trimeric complex formation revealed that trimeric LAG3:FS118:PD-L1 complex concentration was highest at a dose of 10 mg/kg once weekly assuming a BC of 10%. Higher trimeric complex is hypothesized to translate to T cell activation and inhibition of tumour growth.

Combining the above observations, 10 mg/kg once weekly dosing is preferred for future trials.

Deriving from the above data, the alternative of a flat dose of 700 mg once weekly is also proposed. Assuming the average patient weight in a population is 70 kg, a dose of 700 mg once weekly would be equivalent to a dose of 10 mg/kg once weekly. If the actual weight of patients in a population ranges from 35-100 kg, a dose of 700 mg once weekly would be equivalent to a dose in the range of 20 mg/kg to 7 mg/kg once weekly, depending on the patients' actual weight. This would be within the dose range in which stable disease responses were observed without TEAE in the FIH Phase I trial and is therefore expected to be efficacious. A similar rationale can be employed for any particular patient population in question and thus the skilled person, based on the teaching herein, would be able to identify a suitable flat dose for any particular patient population. For instance, if the average weight of patients in a population is estimated to be 80 kg, a dose of 800 mg once weekly would be equivalent to a dose of 10 mg/kg once weekly. If the actual weight of patients in the population ranges from 40-100 kg, a dose of 800 mg once weekly would be equivalent to a dose in the range of 20 mg/kg to 7 mg/kg once weekly, depending on the patients' actual weight. This would again be expected to be efficacious for the reasons explained above.

### Example 7: SCCHN Protocol for Phase I Expansion Trial

In order to explore the clinical activity of FS118 in a specific tumour type, an extension to the Phase I clinical trial is planned. This is called a Phase I expansion cohort and involves recruiting a pre-specified number of patients in order to further assess the safety, pharmacokinetics/pharmacodynamics and clinical efficacy of FS118.

The planned expansion cohort will contain only patients with relapsed or metastatic squamous cell carcinoma of the head and neck (SCCHN). SCCHN was specifically chosen because in the FIH Phase I trial (see Example 2) there were three SCCHN patients, dosed with 3, 10 and 20 mg/kg FS118 once weekly respectively, who remained on study for 26, 15 and 27 weeks respectively (see Example 2.4.1, Table 8) indicating that FS118 may be particularly effective at treating SCCHN. In addition, increased levels of LAG-3 on T cells in the tumour microenvironment of SCCHN patients has previously been observed, as has increased levels of PD-1 (Hanna et al., 2018; Deng et al., 2016) suggesting elevated levels of PD-L1 also. Thus, there is a clear biological rationale for FS118, which targets both LAG-3 and PD-L1, to be efficacious in the SCCHN tumour microenvironment. More specifically, the expansion cohort will contain SCCHN patients who have a disease site of oral cavity, oropharynx, larynx or hypopharynx and are not eligible to receive curative therapies, such as surgery or radiation. Anti-PD-1 antibodies are currently approved by regulatory authorities for these disease sites and thus the patients recruited will have been pre-treated with anti-PD-1 antibodies which is important for the patient recruitment strategy described below. Human papilloma virus (HPV) is thought to cause approximately 20% of SCCHN, especially disease in the oropharynx, known as oropharyngeal cancer. These patients typically have a better clinical outcome in response to anti-cancer treatments than other SCCHN patients where the disease might be caused by tobacco and/or alcohol use. Therefore, the HPV status will be recorded before patients enter the study and if it is not known they will be tested after they have entered the study.

For the reason explained above, all patients will have been previously treated with approved anti-PD-1 antibodies either as a monotherapy or in combination with chemotherapy, and have progressive disease, before they enter the study. The patients must have Acquired resistance to prior PD-1 therapy as defined herein (i.e. patients had a complete or partial response on the prior anti-PD-1 therapy, or showed stable disease for more than 3 months, but then transitioned to progressive disease). This is in view of the inventors' discovery that patients with Acquired resistance are significantly more likely to better respond to FS118 (e.g. by exhibiting stable disease and staying on treatment for longer)—see Example 3.

In order to have had prior treatment with approved anti-PD-1 antibodies, patients must have had PD-L1 levels of >1% either by combined positive score (CPS) or tumour proportion score (TPS), as per the drug labels. Therefore, it is anticipated that all patients who enter the Phase I Expansion trial will have PD-L1 levels >1% and these will be recorded. This is important because the present inventors have demonstrated that baseline PD-L1 levels prior to FS118 treatment in Acquired resistance patients positively correlated with the length of treatment with FS118 (see Example 4). In order for the prior anti-PD-1 therapy to have washed out of each patient's system they must wait a minimum of 28 days before they can enter into the planned Phase I Expansion trial. Further, the patients cannot be more than 12 weeks from the termination of their last anti-PD-1 therapy and the start of treatment with FS118. This ensures that patients who enter the Phase I Expansion trial require immediate further therapy for their progressive disease.

Prior to entry into the Phase I Expansion trial, all patients will be required to provide a biopsy of their tumour as well as blood samples. From the biopsy, baseline PD-L1 and LAG-3 expression levels on tumour cells and T-cells respectively will be measured and analysed, in addition to other features of their cancers such as the percentage of CD8+ T cells within the tumour microenvironment. Blood samples will be used to measure and analyse levels of immune cell populations including Ki67+ immune cells as described in Example 2.3.2.3 and/or soluble LAG-3 or soluble PD-L1 in the plasma. All patients will be dosed with 10 mg/kg FS118 on a once weekly basis consistent with the dose regimen recommendation described in Example 6. The efficacy of FS118 in SCCHN will be evaluated using a clinical endpoint of disease control rate (DCR) after 24 weeks on treatment. This is the percentage of patients who have a complete response (CR), partial response (PR) and/or stable disease (SD) over a 24-week period starting from the initiation of treatment with FS118. Thus, this would cover a patient who, for example, first exhibits a partial response but then moves to stable disease or vice versa. Patients who receive standard of care therapy after anti-PD-1 therapy (for example: taxanes such as docetaxel or paclitaxel, cetuximab or methotrexate) would typically have a DCR rate at 24 weeks of <20% based on principal investigators' experience. This means that >80% of such patients would have progressive disease by 24 weeks from the start of the standard of care therapy. It is the minimum objective that FS118 exceed said DCR rate for standard of care therapies administered after an anti-PD-1 therapy. Given that the patients in the Phase I Expansion trial will be less highly pre-treated than those in the FIH and dose escalation Phase I clinical trials (see Example 2), this objective is considered achievable.

The statistical design of the Phase I Expansion trial will utilise a method called a Simon's 2-stage minimax design (Simon, 1989). At the first stage, 10 patients will be recruited. If 1 or none of said 10 patients achieve disease control (CR, PR and/or SD) over a 24-week period from initiation of treatment with FS118, enrolment will terminate and FS118 will not be deemed sufficiently efficacious compared to standard of care therapies to warrant continuing recruitment. Otherwise, a further 12 patients will be enrolled as a second stage. Upon completion of the second stage, if 6 or more patients out of 22 evaluable patients achieve disease control (CR, PR and/or SD) over a 24-week period from initiation of treatment with FS118 then FS118 will deemed to be efficacious in these patients.

In order to further understand which patients might benefit the most from treatment with FS118, the expression levels of PD-L1 and LAG-3 in the patients' cancers (recorded at baseline using the mandatory biopsy material), will be compared against clinical benefit (CR, PR and/or SD during the study and length of time on treatment) to FS118 to identify if any correlations exist. Additionally, changes in the levels of soluble LAG-3 in patient plasma samples and in the frequency and Ki67 expression levels of peripheral immune cell populations will also be monitored as pharmacodynamic markers of a response to FS118.

Sequence listing
Amino acid sequence of the heavy chain of anti-human LAG-3/PD-L1
mAb² FS118 (with LALA mutation)
CDRs are underlined. The AB, CD, and EF loop sequences are shown in
bold and underlined.
(SEQ ID NO: 1)
EVQLVESGGGLVQPGRSLRLSCAASGFTFDDYAMHWVRQTPGKGLEWVSGISWKSNIIGYADSVKG
RFTISRDNAKNSLYLQMNSLRAEDTALYYCARDITGSGSYGWFDPWGQGTLVTVSSASTKGPSVFPL
APSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKAL
PAPIEKTISKAKGQPREPQVYTLPPSWDEPWGEDVSLTCLVKGFYPSDIAVEWESNGQPENNYKTTP
PVLDSDGSFFLYSKLTVPYDRWVWPDEFSCSVMHEALHNHYTQKSLSLSPG
Amino acid sequence of the liqht chain of anti-human LAG-3/PD-L1
mAb² FS118
CDRs are underlined.
(SEQ ID NO: 2)
DIQMTQSPSSLSASVGDRVTITCRASQSISSYLNWYQQKPGKAPKPLIYVASSLQSGVPSSFSGSGS
GTDFTLTISSLQPEDFATYYCQQSYSNPITFGQGTRLEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLS
SPVTKSFNRGEC
Amino acid sequence of the heavy chain of anti-mouse LAG-3/PD-L1 mAb²
FS18-7-108-29/S1 (with LALA mutation)
CDRs are underlined. Position of the AB, CD, and EF loop sequences
are shown in bold and underlined. The position of LALA mutation is
shown in bold.
(SEQ ID NO: 3)
EVQLVESGGGLVQPGGSLRLSCAASGFTFSDSWIHWVRQAPGKGLEWVAWISPYGGSTYYADSVK
GRFTISADTSKNTAYLQMNSLRAEDTAVYYCARRHWPGGFDYWGQGTLVTVSAASTKGPSVFPLAP
SSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSWDEPWGEDVSLTCLVKGFYPSDIVVEWESNGQPENNYKTTPP
VLDSDGSFFLYSKLTVPFERWMWPDEFSCSVMHEALHNHYTQKSLSLSPG
Amino acid sequence of the liqht chain of anti-mouse LAG-3/PD-L1 mAb²
FS18-7-108-29/S1
CDRs are underlined.
(SEQ ID NO: 4)
DIQMTQSPSSLSASVGDRVTITCRASQDVSTAVAWYQQKPGKAPKLLIYSASFLYSGVPSRFSGSGS
GTDFTLTISSLQPEDFATYYCQQYLFTPPTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLS
SPVTKSFNRGEC
Amino acid sequence of the heavy chain of anti-FITC mAb G1AA/4420
(comprising LALA mutation)
Position of the CDRs are underlined. Position of LALA mutation is
in bold.
(SEQ ID NO: 5)
EVKLDETGGGLVQPGRPMKLSCVASGFTFSDYWMNWVRQSPEKGLEWVAQIRNKPYNYETYYSDS
VKGRFTISRDDSKSSVYLQMNNLRVEDMGIYYCTGSYYGMDYWGQGTSVTVSSASTKGPSVFPLAP
SSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
Amino acid sequence of the anti-FITC mAb G1AA/4420 light chain
Position of the CDRs are underlined.
(SEQ ID NO: 6)
DVVMTQTPLSLPVSLGDQASISCRSSQSLVHSNGNTYLRWYLQKPGQSPKVLIYKVSNRFSGVPDRF
SGSGSGTDFTLKISRVEAEDLGVYFCSQSTHVPWTFGGGTKLEIKRTVAAPSVFIFPPSDEQLKSGTA
SVVCLLNNFYPREAKVQWKVDNALQSGNSQESVTEQDSKDSTYSLSSTLTLSKADYEKHKVYACEV
THQGLSSPVTKSFNRGEC

### REFERENCES

All documents mentioned in this specification are incorporated herein by reference in their entirety.
Chirmule et al., Immunogenicity to Therapeutic Proteins: Impact on PK/PD and Efficacy, The AAPS Journal, 2012, 14:296-302
D'Argenio D Z, Schumitzky A, Wang X. ADAPT 5 User's Guide: Pharmacokinetic/Pharmacodynamic Systems Analysis Software. Los Angeles: Biomedical Simulations Resource; 2009.
Deng et al., LAG-3 confers poor prognosis and its blockade reshapes antitumor response in head and neck squamous cell carcinoma, Oncoimmunology, 2016, 5(11):e1239005
Eisenhauer et al., New response evaluation criteria in solid tumours: Revised RECIST guideline (version 1.1), European Journal of Cancer, 2009, 28-247
Fujita et al., Retreatment With Anti-PD-L1 Antibody in Advanced Non-small Cell Lung Cancer Previously Treated With Anti-PD-1 Antibodies, Anticancer Res, 2019, 39(7):3917-3291
Fujita et al., Retreatment with anti-PD-1 antibody in non-small cell lung cancer patients previously treated with anti-PD-L1 antibody, Thoracic Cancer, 2019, 1759-7706
Hanna et al., Frameshift events predict anti-PD-1/L1 response in head and neck cancer, JCI Insight, 2018, 3(4):e98811
Heery C R, O'Sullivan-Coyne G, Madan R A et al. Avelumab for metatstatic or locally advanced previously treated solid tumours (JAVELIN Solid Tumour): a phase 1a, muticohort, dose-escalation trial. The Lancet Oncol 2017; 18(5): 587-598
Herbst R, Soria J-C, Kowanetz M et al. Predictive correlates of response to the anti-PD-L1 antibody MPDL3280A in cancer patients. Nature 2014; 515(7528): 563-567
Marin-Acevedo et al. Next generation of immune checkpoint therapy in cancer: new developments and challenges. Journal of Hematology & Oncology (2018); 11:39
Martini et al., Response to single agent PD-1 inhibitor after progression on previous PD-1/PD-L1 inhibitors: a case series, J. Immunotherapy Cancer, 2017, 5:66
Morgado et al., Simultaneous measurement and significance of PD-1, LAG-3 and TIM-3 expression in human solid tumors, Cancer Res., 2018, 78 (13 Suppl):Abstract nr 1681
Nowicki et al., Mechanisms of Resistance to PD-1 and PD-L1 Blockade, The Cancer Journal, 2018, 24(1):47-53
Petitprez et al., B cells are associated with survival and immunotherapy response in sarcoma, Nature, 2020, 577(7791):556-560
Saber H et al. An FDA oncology analysis of immune activating products and first-in-human dose selection. Regulatory Toxicology and Pharmacology 2016; 81: 448-456.
Sharma et al., Primary, Adaptive and Acquired Resistance to Cancer Immunotherapy. Cell, 2017, 168(4):707-723
Seymour et al., iRECIST: guidelines for response criteria for use in trial testing immunotherapeutics, Lancet Oncol., 2017, 18(3):e143-e152
Simon R, Optimal Two-Stage Designs for Phase II Clinical Trials, Controlled Clinical Trials, 1989, 10:1-10

1. An antibody molecule which binds programmed death-ligand 1 (PD-L1) and lymphocyte-activation gene 3 (LAG-3) for use in a method of treating cancer in a human patient,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of 3 mg to 20 mg per kg of body weight of the patient.

2. A method of treating cancer in a human patient, wherein the method comprises administering to the patient a therapeutically effective amount of an antibody molecule which binds PD-L1 and LAG-3,
wherein the antibody molecule comprises the heavy chain sequence set forth in SEQ ID NO: 1 and the light chain sequence set forth in SEQ ID NO: 2; and
wherein the method comprises administering the antibody molecule to the patient once weekly at a dose of 3 mg to 20 mg per kg of body weight of the patient.

3. The antibody molecule for use, or method, according to claim 1 or 2, wherein the method comprises administering the antibody molecule at a dose of 10 mg to 20 mg per kg of body weight of the patient.

4. The antibody molecule for use, or method, according to any one of claims 1 to 3, wherein the method comprises administering the antibody molecule at a dose of 10 mg per kg of body weight of the patient.

5. The antibody molecule for use, or method, according to claim 1 or 2, wherein the method comprises administering the antibody molecule to the patient at a dose of 210 mg to 1400 mg.

6. The antibody molecule for use, or method, according to any one of claim 1 to 3, or 5, wherein the method comprises administering the antibody molecule to the patient at a dose of 700 mg to 1400 mg.

7. The antibody molecule for use, or method, according to any one of claims 1 to 6, wherein the method comprises administering the antibody molecule to the patient at a dose of 700 mg.

8. The antibody molecule for use, or method, according to any one of claims 1 to 7, wherein the tumour is refractive to treatment with one or more checkpoint inhibitors, has relapsed during or following treatment with one or more checkpoint inhibitors, or is responsive to treatment with one or more checkpoint inhibitors.

9. The antibody molecule for use, or method, according to claim 8, wherein the immune checkpoint inhibitor is a programmed cell death protein 1 (PD-1) or PD-L1 inhibitor.

10. An antibody molecule which binds PD-L1 and LAG-3 for use in a method of treating cancer in a human patient who has been subjected to treatment with a prior anti-PD-1 or anti-PD-L1 therapy, the antibody molecule comprising the heavy chain sequence set forth in SEQ ID NO: 1 and the light chain sequence set forth in SEQ ID NO: 2;
wherein a tumour of the patient has been determined to have an acquired resistance phenotype in respect of the prior anti-PD-1 or anti-PD-L1 therapy, and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

11. A method of treating cancer in a human patient who has been subjected to treatment with a prior anti-PD-1 or anti-PD-L1 therapy, the method comprising administering to the patient a therapeutically effective amount of an antibody molecule which binds PD-L1 and LAG-3 and comprises the heavy chain sequence set forth in SEQ ID NO: 1 and the light chain sequence set forth in SEQ ID NO: 2;
wherein a tumour of the patient has been determined to have acquired resistance phenotype in respect of the prior anti-PD-1 or anti-PD-L1 therapy, and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy.

12. The antibody molecule for use, or method, according to any one of claims 10 to 11, wherein at least 15% of tumour cells in a sample of the tumour obtained from the patient prior to treatment with the antibody have been determined to be PD-L1 positive.

13. A method of determining whether a cancer patient who has been subjected to treatment with a prior anti-PD-1 or anti-PD-L1 therapy is likely to respond to treatment with an antibody molecule which binds PD-L1 and LAG-3 and comprises the heavy chain sequence set forth in SEQ ID NO: 1 and the light chain sequence set forth in SEQ ID NO: 2,
the method comprising determining whether a tumour of the patient has an acquired resistance phenotype or primary resistance phenotype in respect of the prior anti-PD-1 or anti-PD-L1 therapy,
wherein a tumour with an acquired resistance phenotype has a higher likelihood of responding to treatment with the antibody than a tumour with a primary resistance phenotype; and
wherein a tumour with an acquired resistance phenotype is a tumour which showed a complete or partial response to treatment with the prior anti-PD-1 or anti-PD-L1 therapy, or showed stable disease for more than 3 months whilst subjected to treatment with the prior anti-PD-1 or anti-PD-L1 therapy, and
a tumour with a primary resistance phenotype is a tumour which achieved stable disease for 3 months or less whilst subjected to treatment with the prior anti-PD-1 or anti-PD-L1 therapy, including a tumour with a best overall response of progressive disease.

14. The method according to claim 13, the method further comprising determining whether at least 15% of tumour cells in a sample of the tumour obtained from the patient prior to treatment with the antibody are PD-L1 positive,
wherein a tumour with an acquired resistance phenotype comprising at least 15% PD-L1 positive tumour cells has a higher likelihood of responding to treatment with the antibody than a tumour with a primary resistance phenotype, or a tumour with an acquired resistance phenotype comprising less than 15% PD-L1 positive tumour cells.

15. The antibody molecule for use, or method, according to any one of claims 1 to 14, wherein the cancer is selected from the list consisting of: squamous cell carcinoma of the head and neck (SCCHN), gastric cancer, oesophageal cancer, non-small cell lung cancer (NSCLC), mesothelioma, melanoma, prostate cancer, bladder cancer, breast cancer, colorectal cancer (CRC), adenocarcinoma of the esophagogastric junction (GEJ), renal cell carcinoma (RCC), hepatocellular carcinoma (HCC), small-cell lung cancer (SCLC), uterine cancer, vulvar cancer, testicular cancer, penile cancer, leukaemia, Merkel cell carcinoma and nasopharyngeal cancer.

16. The antibody molecule for use, or method, according to any one of claims 1 to 14, wherein the cancer is selected from the list consisting of: sarcoma, thyroid cancer, glioblastoma multiforme (GBM), ovarian cancer, basal cell carcinoma, MSI-H solid tumours, triple negative breast cancer (TNBC), cervical cancer, oesophageal cancer, multiple myeloma (MM), pancreatic cancer, meningioma, endometrial cancer, thymic carcinoma, gestational trophoblastic neoplasia, lymphomas, peritoneal carcinomatosis, microsatellite stable (MSS) colorectal cancer, and gastrointestinal stromal tumours (GIST).

17. The antibody molecule for use, or method, according to any one of claims 1 to 14, wherein the cancer is selected from the list consisting of: head and neck cancer, gastric cancer, oesophageal cancer, NSCLC, mesothelioma, cervical cancer, thyroid cancer and soft-tissue sarcoma; preferably wherein the head and neck cancer is SCCHN.

18. The antibody molecule for use, or method, according to any one of claims 1 to 14, wherein the cancer is head and neck cancer, preferably SCCHN.

19. The antibody molecule for use, or method, according to claim 18, wherein the SCCHN disease site is the oral cavity, oropharynx, larynx or hypopharynx.

20. The antibody molecule for use, or method, according to claim 18 or 19 wherein the method comprises administering the antibody molecule to the patient once weekly at a dose of 10 mg per kg of body weight of the patient.

21. The antibody molecule for use, or method, according to any one of claims 1 to 20, wherein the antibody molecule is administered intravenously.


SEQUENCE LISTING (6 sequences)

SEQ ID NO: 1 - protein, 451 aa, Artificial Sequence
  heavy chain of anti-human LAG-3/PD-L1 mAb2 FS118 (with LALA mutation)
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSWDEPWGEDVSLTCLVKGFYPSDIAVEWESNGQPENNYKTTPPVLDSDGSFFLYSKLTVPYDRWVWPDEFSCSVMHEALHNHYTQKSLSLSPG

SEQ ID NO: 2 - protein, 214 aa, Artificial Sequence
  light chain of anti-human LAG-3/PD-L1 mAb2 FS118
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Artificial Sequence
  heavy chain of anti-mouse LAG-3/PD-L1 mAb2 FS18-7-108-29/S1 (with LALA mutation)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSWDEPWGEDVSLTCLVKGFYPSDIVVEWESNGQPENNYKTTPPVLDSDGSFFLYSKLTVPFERWMWPDEFSCSVMHEALHNHYTQKSLSLSPG

SEQ ID NO: 4 - protein, 214 aa, Artificial Sequence
  light chain of anti-mouse LAG-3/PD-L1 mAb2 FS18-7-108-29/S1
DIQMTQSPSSLSASVGDRVTITCRASQDVSTAVAWYQQKPGKAPKLLIYSASFLYSGVPSRFSGSGSGTDFTLTISSLQPEDFATYYCQQYLFTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 447 aa, Artificial Sequence
  heavy chain of anti-FITC mAb G1AA/4420 (comprising LALA mutation)
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, Artificial Sequence
  anti-FITC mAb G1AA/4420 light chain
DVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC